(19) United States
(12) Patent Application Publication
(10) Pub. No.: US 2005/0239767 A1
(43) Pub. Date: Oct. 27, 2005

(54) Intermolecular SNAr of the heterocycle-activated nitro and fluoro groups-application in the synthesis of polyazamacrocyclic ligands

(76) Inventors: Michael K. Chan, Hilliard, OH (US); Tomasz Fekner, Lublin (PL)

Correspondence Address: CALFEE HALTER & GRISWOLD, LLP, 800 SUPERIOR AVENUE, SUITE 1400, CLEVELAND, OH, 44114, US

(21) Appl. No.: 10/978,082

(22) Filed: Oct. 28, 2004

Related U.S. Application Data
(60) Provisional application No. 60/514,996, filed on Oct. 28, 2003.

(51) Int. Cl. A61K 31/553; A61K 31/551; H01L 51/40; C07F 9/80; C07D 491/04; C07D 487/04

(52) U.S. Cl. 514/211.1; 540/546; 540/556; 548/102; 548/111; 514/219; 438/99

(57) ABSTRACT

A new class of tetracylic benzimidazole compounds and derivatives thereof. Additionally provided is a synthetic route for the generation of these and related compounds via Intramolecular Aromatic Nucleophilic Substitution (S_{N}Ar) of the Benzimidazole-Activated Nitro Groups. Additionally, a facile route for the generation of novel phenol species as thermal decomposition of compounds the S_{N}Ar product, which occurs at high temperature resulting in cleavage of the ether linkage and formation of a vinyl group and phenol is provided. Also provided are methods of using the compounds described herein in the treatment HIV.

### CROSS-REFERENCE TO RELATED APPLICATIONS

This application claims priority to U.S. Provisional Patent Application No. 60/514,996 filed Oct. 28, 2003, entitled, “Intermolecular SNAr of the Heterocycle-Activated Nitro and Fluoro Groups—Application in the Synthesis of Polyazamacrocyclic Ligands,” the entirety of which is incorporated herein by reference.

### STATEMENT ON FEDERALLY FUNDED RESEARCH

This work was supported at least in part by the National Science Foundation (CAREER Award No. 9984071). The government may have certain rights in this invention.

### SUMMARY OF THE INVENTION

New compounds of the formula: 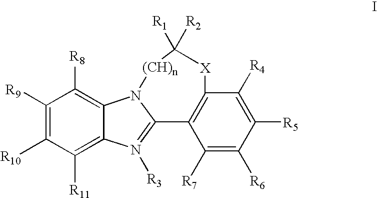
wherein:
R₁ and R₂ can be the same or different and are selected from the group consisting of H, linear alkyl, branched alkyl, cycloalkyl, vinyl, alkenyl, alkynyl, hydroxyl, halide, nitro, carboxylate, amino, amido, epoxide, and labeling reagents and fluorescent tags, such as biotin, coumarin and fluoroscene dyes;
R₃ is optional and may be null (i.e. no group), an oxo, a terminal epoxide, alkyl, branched alkyl, cycloalkyl, hydroxyl, halide, nitro, carboxylate, amido, epoxide, amino, substituted amino, aryl, or vinyl group;
R₄-R₇ can be the same or different and are selected from the group consisting of H, linear alkyl, branched alkyl, cycloalkyl, hydroxyl, halide, nitro, carboxylate, amido, epoxide, amino, substituted amino, aryl, vinyl, acetal, aldehyde, or a labeling reagent such as biotin, coumarin or fluoroscene;
R₈-R₁₁ can be the same or different and are selected from the group consisting of H, linear alkyl, branched alkyl, cycloalkyl, hydroxyl, halide, nitro, carboxylate, amido, epoxide, amino, substituted amino, aryl, acetal, aldehyde, or vinyl group;
X is a heteroatom selected from O, S, Se, NH, PH, AsCH₂, activated CH₂ or wherein the hydrogens in NH, PH, AsCH₂ may be substituted with other groups, such as lower alkyl;
n is 0, 1, 2, 3, 4, 5. Provided also are derivatives thereof.

### BRIEF DESCRIPTION OF THE FIGURES

FIG. 1 shows HIV-screening data for a sample of a compound disclosed herein.

FIG. 2 shows HIV-screening data for a sample of a compound disclosed herein.

FIG. 3 shows HIV-screening data for a sample of a compound disclosed herein.

FIG. 4 shows HIV-screening data for a sample of a compound disclosed herein.

### DETAILED DESCRIPTION OF THE INVENTION

A wide range of 2-(2-nitrophenyl)-1H-benzimidazoles undergo high-yielding intramolecular S_{N}Ar of nitrite with N-pendant alkoxides under mild conditions (DMF, rt). When this operationally simple process is carried out at elevated temperature in the presence of excess NaH, the initially formed S_{N}Ar products are converted to the corresponding N-vinyl-substituted 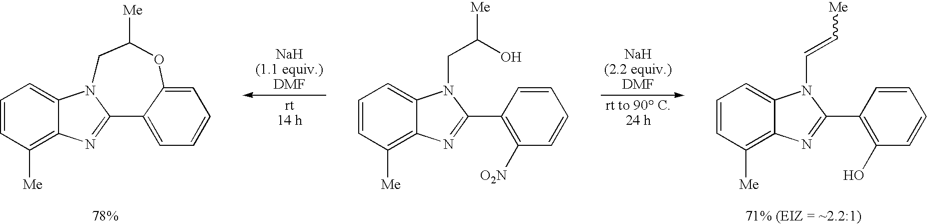
2-(2-hydroxyphenyl)-1H-benzimidazoles via base-catalyzed isomerization.

Provided herein is a new class of tetracylic benzimidazole compounds which may be formed by the above reaction. Additionally provided is a facile synthetic route for the generation of these and related compounds via Intramolecular Aromatic Nucleophilic Substitution (S_{N}Ar) of the Benzimidazole-Activated Nitro Groups. Additionally, a facile route for the generation of novel phenol species as thermal decomposition of compounds of formula I, which occurs at high temperature resulting in cleavage of the ether linkage and formation of a vinyl group and phenol is provided. Also provided are methods of using the compounds described herein in the treatment of various kinds of cancer and HIV.

The compounds described herein are shown in the general formula below: 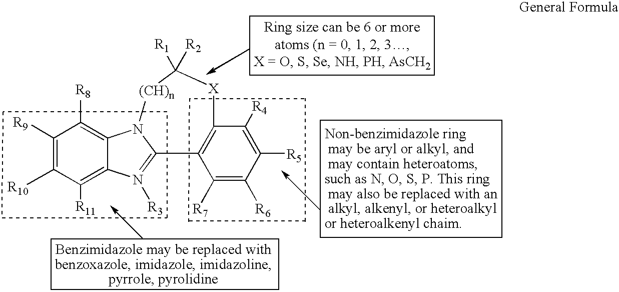
wherein:
R₁ and R₂ can be the same or different and are selected from the group consisting of H, linear alkyl, branched alkyl, cycloalkyl, vinyl, alkenyl, alkynyl, hydroxyl, halide, nitro, carboxylate, amino, amido, epoxide, and labeling reagents and fluorescent tags, such as biotin, coumarin and fluoroscene dyes;
R₃ is optional and may be null (i.e. no group), an oxo, a terminal epoxide, alkyl, branched alkyl, cycloalkyl, hydroxyl, halide, nitro, carboxylate, amido, epoxide, amino, substituted amino, aryl, or vinyl group;
R₄-R₇ can be the same or different and are selected from the group consisting of H, linear alkyl, branched alkyl, cycloalkyl, hydroxyl, halide, nitro, carboxylate, amido, epoxide, amino, substituted amino, aryl, vinyl acetal, aldehyde, or a labeling reagent such as biotin, coumarin or fluoroscene;
R₈-R₁₁ can be the same or different and are selected from the group consisting of H, linear alkyl, branched alkyl, cycloalkyl, hydroxyl, halide, nitro, carboxylate, amido, epoxide, amino, substituted amino, aryl, acetal, aldehyde, or vinyl group;
X is a heteroatom selected from O, S, Se, NH, PH, AsCH₂, activated CH₂ or wherein the hydrogens in NH, PH, AsCH₂ may be substituted with other groups, such as lower alkyl;
Z is carbon or a heteroatom selected from N and O, and may be the same or different in a single embodiment; in one embodiment, both Z are N;
n is 0, 1, 2, 3, 4, 5.

Additionally, any of the carbons of the non-benzimidazole aryl group may be replaced with a heteroatom provided that the 2-position of the ring is aromatic or heteroaromatic with a preferred ring size of 5 to 6 atoms, though others ring sizes are viable. Possible heteroatoms include N, O, S, and P atoms. In another embodiment, the non-benzimidazole aryl group may be replaced with another an alkyl, cycloalkenyl or heteroalkenyl, provided that there is an sp² hybridized atom at the 2-position. Furthermore, the benzimidazole structure may be replaced by a structure selected from indole (Formula III), benzoxazole (Formula IV), imidazole (Formula V), imidazoline (Formula VI), pyrrole (Formula VII), or pyrrolidine (Formula VIII). 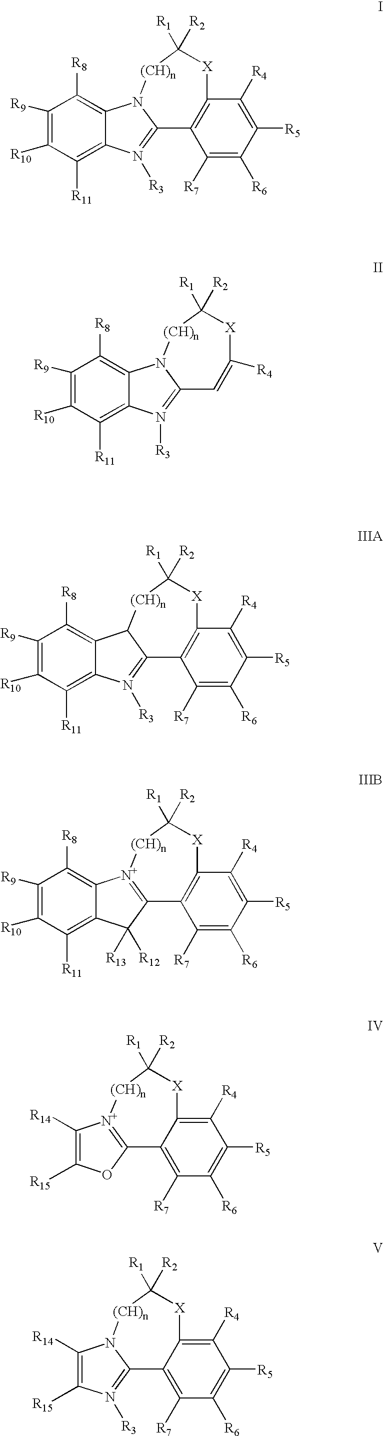 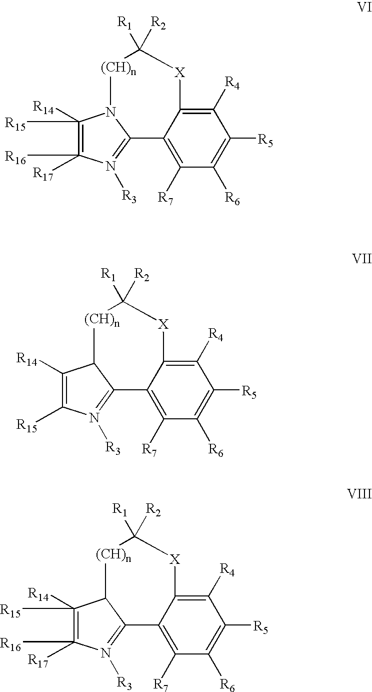
wherein:
R₁ and R₂ can be the same or different and are selected from the group consisting of H, linear alkyl, branched alkyl, cycloalkyl, vinyl, alkenyl, alkynyl, hydroxyl, halide, nitro, carboxylate, amino, amido, epoxide, and labeling reagents and fluorescent tags, such as biotin, coumarin and fluoroscene dyes;
R₃ is optional and may be null (i.e. no group), an oxo, a terminal epoxide, alkyl, branched alkyl, cycloalkyl, hydroxyl, halide, nitro, carboxylate, amido, epoxide, amino, substituted amino, aryl, or vinyl group;
R₄-R₇ can be the same or different and are selected from the group consisting of H, linear alkyl, branched alkyl, cycloalkyl, hydroxyl, halide, nitro, carboxylate, amido, epoxide, amino, substituted amino, aryl, vinyl, acetal, aldehyde, or a labeling reagent such as biotin, coumarin or fluoroscene;
R₈-R₁₁ can be the same or different and are selected from the group consisting of H, linear alkyl, branched alkyl, cycloalkyl, hydroxyl, halide, nitro, carboxylate, amido, epoxide, amino, substituted amino, aryl, acetal, aldehyde, or vinyl group;
R₁₄-R₁₇ can be the same or different and are selected from the group consisting of H, linear alkyl, branched alkyl, cycloalkyl, hydroxyl, halide, nitro, carboxylate, amido, epoxide, or amino; and wherein these groups may contain further substitutions.
X is a heteroatom selected from O, S, Se, NH, PH, AsCH₂, activated CH₂ or wherein the hydrogens in NH, PH, AsCH₂ may be substituted with other groups, such as lower alkyl;
n is 0, 1, 2, 3, 4, 5.

An interesting aspect of the compounds described herein is that the cyclic ether that is formed can vary in size from a six atom ring to larger rings, as shown in the formulae above. In many embodiments, the compounds are cyclic ethers, wherein X is O; however, in other embodiments, other heteroatoms are present instead (S, NH, PH, even activated CH₂).

In some embodiments, the compounds may comprise an indole (Formulae IIIA and IIIB) with possible substituents R₁₂ and R₁₃ groups consisting of H, linear alkyl, branched alkyl, cycloalkyl, hydroxyl, halide, nitro, carboxylate, amido, epoxide, amino, or substituted amino group. In another embodiment, the compounds may comprise a benzoxazole (Formulae III and IV, respectively).

In other embodiments, the compounds may comprise an imidazole, imidazoline, pyrrole, or pyrrolidine, with possible substituents R₁₄-R₁₇ groups consisting of H, linear alkyl, branched alkyl, cycloalkyl, hydroxyl, halide, nitro, carboxylate, amido, epoxide, amino, or substituted amino group (Formulae V, VI, VII, and VIII respectively).

The compounds described herein may be neutral, or may be also be substituted and both benzimidazole N¹ and N³ nitrogens, thus giving a cationic species.

Also provided are methods of making the new compounds described herein. The method comprises the step of allowing a compound of formula IX to undergo intramolecular S_{N}Ar of with NaH in DMF for a time sufficient for the reaction to occur. The reaction occurs at room temperature.

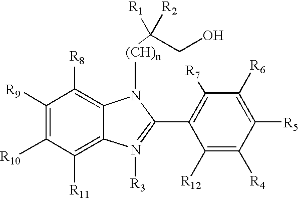

The functional groups for formula IX will be the same as described above except now the leaving group, R₁₂ is part of the structure and is selected from the group consisting of —NO₂, F, Cl, Br, OTs, SOPH, N₃. Other suitable starting materials corresponding to Formulae III-VIII are also encompassed in the methods described herein.

Further provided are pharmaceutical preparations comprising the compounds described herein as well as pharmaceutically acceptable salts and metabolites thereof. The compounds described herein have been confirmed active in in vitro anti-HIV drug screening tests.

Also provided is the use of the chemistry described and the benzimidazoles formed using this chemistry for the generation of second and third order non-linear optical materials.

Also provided is a post S_{N}Ar process which leads to overall substitution of the nitro group with OH, i.e., conversion of nitroarenes to phenols. The process comprises the step of treating a compound of formula I-IX with excess NaH in DMF at an elevated temperature for a time sufficient to convert the nitroarene to the corresponding phenol. The post S_{N}Ar reaction is generally carried out for approximately 24 hours for maximal conversion, however, one of ordinary skill in the art would be able to determine an appropriate length of time to carry out this reaction for a particular starting material. The post S_{N}Ar reactions are generally carried out at temperatures ranging from room temperature to about 90° C., depending on factors such as steric factors from group R₁, and can readily be determined by those of ordinary skill in the art.

The compounds described herein are useful for a variety of purposes ranging from the generation of homochiral ligands for asymmetric catalysis, novel materials for chiral separations, and for pharmaceutical use. Both neutral and cationic species can also be prepared made using the methods described herein. Like their neutral counterparts, these cationic and anionic compounds may useful for the generation of catalysts and materials, and for the design of potential drugs, via the generation and screening of combinatorial libraries.

The compounds described herein, with the exception of the post S_{N}Ar compounds, comprise a fused four-ring structure—a benzimidazole or similar structure, an aryl, and a cyclic ether. These compounds are steroid analogs and may be useful in treatments for which steroids are also often used, such as cancer and HIV, for example. These compounds may also be used as inhibitors of various biological targets including: steroid receptors, proteins involved in steroid biosynthesis, and a variety of nucleic acid and nucleotide binding proteins. Additionally, the planarity of these compounds may enable them to serve as possible DNA intercators.

The compounds described herein are also expected to be useful in the treatment or prophylaxis of cancer, either as anti-cancer agents or as adjuvants to chemotherapy or radiation therapy in the treatment of cancer. Their anti-cancer activity has been shown in in vitro studies. These compounds are also expected to have antibacterial properties, making them useful in the treatment of bacterial infections in humans and animals, as well as in other applications wherein anti-bacterial properties are desired.

In the course of our ongoing studies on the synthesis of configurationally stable, highly ruffled, cyclic bis(benzimidazole) ligands,^{i,ii} we required etherification of alcohol 1 (Scheme 1). Somewhat unexpectedly, its treatment with NaH in DMF, followed by the addition of 1,8-dibromooctane, did not give the desired bis(ether) 2. Instead, the seven-membered cyclic ether 3, possessing a novel, tetracyclic 6,7-dihydro-5-oxa-7a,12-diazadibenzo[a,e]azulene skeleton, was formed in excellent yield as the only isolable product, apparently via an intramolecular S_{N}Ar of the nitro group.

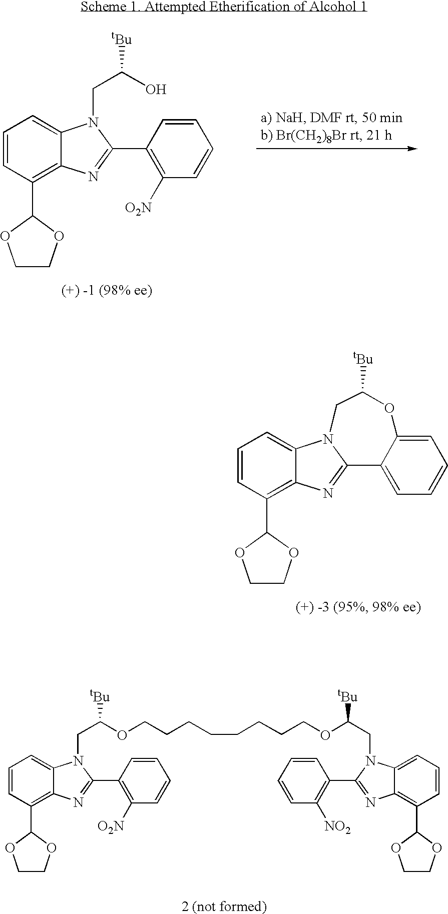

As benzimidazole is an important scaffold in drug discovery, with many of its analogues being used in.the treatment of various viral, bacterial, and fungal infections,ⁱⁱⁱ we were surprised to find that the activating properties of benzimidazole for promoting S_{N}Ar reactions have not been frequently utilized. To the best of our knowledge, the only examples of benzimidazole-activated S_{N}Ar transformations have been reported by Hedrick and co-workers. These reactions involve the high-temperature intermolecular replacement of fluoride by phenoxides in the preparation of thermally stable polymers.^{iv}

The new transformation depicted in Scheme 1 proved quite general, and a series of structurally diverse analogues of alcohol 1^{v} were shown to be competent substrates (Table 1).^{vi} As can be noted, the steric hindrance on the nucleophilic arm is well tolerated, and both moderately (entries 1-2) and severely (entries 3-6) sterically hindered secondary alcohols undergo the cyclization in high yield. In addition, tertiary alcohol 14 (entry 7) undergoes a smooth nitro-group displacement to give the cyclized product 15 in good yield. Substitution ortho to the nitro group, however, gives mixed results. Although the nitro group in benzimidazole 16 (entry 8) undergoes the displacement with high yield, replacement of the chloro substituent with a methyl group (entry 9) has a detrimental effect on the yield of the cyclized product 19. This result can be attributed to both the unfavorable steric and electronic contributions of the methyl group that hinders formation of the intermediate Meisenheimer complex, and also decreases its stability.

TABLE 1
Intramolecular Replacement of the Benzimidazole-Activated Nitro Group^{a}

| | | | yield |
|---|---|---|---|
| entry | substrate | product | (%)^{b} |
| | 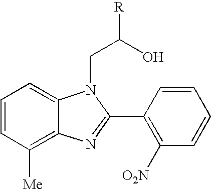 | 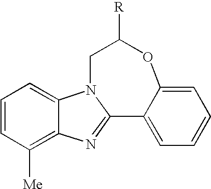 | |
| 1 | 4: R = Me | 5 | 78 |
| 2 | 6: R = n-Bu | 7 | 75 |
| | 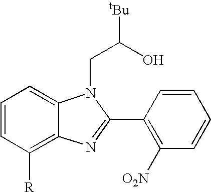 | 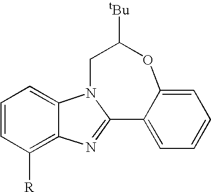 | |
| 3 | 8: R = H | 9 | 86 |
| 4 | 10: R = Me | 11 | 96 |
| 5 | 12: R = CO₂Et | 13 | 89 |
| 6 | 1: R = [1,3]- | 3 | 92 |
| | dioxolane-1-yl | | |
| 7 | 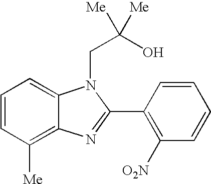 | 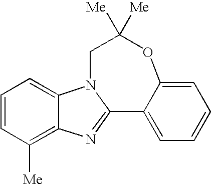 | 68 |
| | 14 | 15 | |
| | 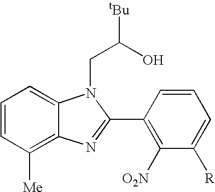 | 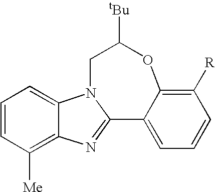 | |
| 8 | 16: R = Cl | 17 | 80 |
| 9 | 18: R = Me | 19 | 7 |

^{a}The reactions were performed in DMF in the presence of NaH (1.1 equiv.).

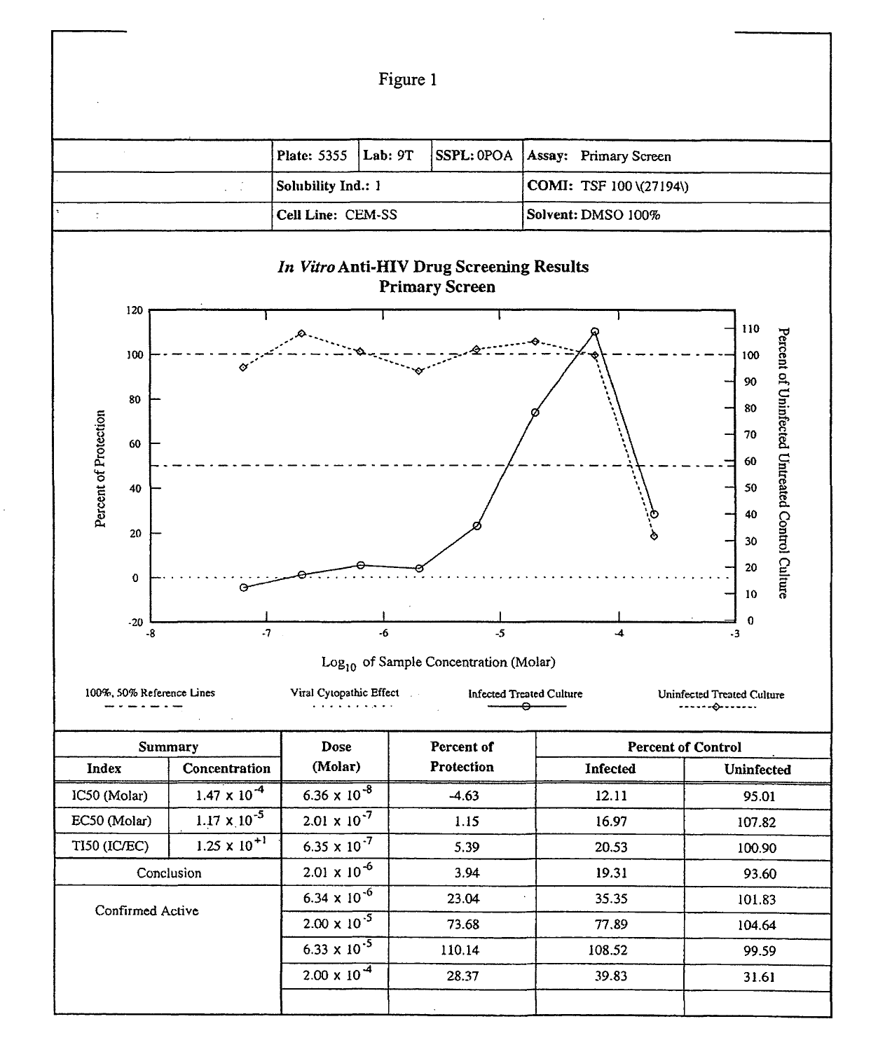

Single crystals of the cyclic ether 9 suitable for X-ray analysis were grown by slow evaporation of its CH₂Cl₂-petroleum ether solution. As anticipated (FIG. 1), the three-atom bridge spanning the two aromatic subunits forces them into a nearly perfect co-planarity (the dihedral angle N₂—C₇—C₈—C₉: 1.4 and 10.9°, respectively, for the two enantiomeric molecules in asymmetric unit). This conformational constraint, common to all the studied seven-membered cyclization products, has a marked effect on the ¹H NMR chemical shift of the aromatic proton located ortho to the aryl-heteroaryl axis.^{vii} As this proton is placed directly within the deshielding region of the benzimidazole aromatic ring current, it is subject to a significant downfield shift compared to the remaining aromatic protons. Indeed, the presence of a significantly downshifted proton signal in the ¹H NMR spectrum can be used to confirm the intramolecular nitro replacement reaction.

To determine whether there is a preferred ring size for this S_{N}Ar cyclization, diol 20 (Scheme 2) was studied.^{viii} For this compound, two modes of cyclization are plausible. One involves the displacement of the nitro group by the sterically less hindered primary alkoxide with the formation of the eight-membered cyclic ether 21, whereas the other involves an analogous displacement by the sterically more encumbered secondary alkoxide leading to ether 22. As the rotation about the aryl-heteroaryl axis in the latter compound is expected to be more severely restricted, it is presumably thermodynamically less favored than its eight-membered analogue 21. It was subsequently experimentally demonstrated that diol 20, when subjected to the standard reaction conditions, is converted exclusively to the seven-membered cyclic ether 22 in high yield. This result presumably reflects an overwhelming kinetic preference for the formation of the smaller of the two possible rings. The identity of the cyclized product 22 was unambiguously confirmed by its conversion, via the corresponding tosylate, into benzimidazole 23. ¹H NMR analysis of compound 23 indicated the presence of an aliphatic methyl group at 1.46 ppm (d, J=6.5 Hz) that corresponds to the hydroxymethyl group in benzimidazole 22. This proved the involvement of the secondary alkoxide in the cyclization of diol 20.

It was expected, however, that this strong preference for the 7-membered product could be altered by introduction of additional steric bulk ortho to the nitro group. When diol 24^{viii} (Scheme 3) was subjected to the standard reaction conditions, the seven-membered cyclic ether 25 was the major product (70%), but a small amount (8%) of its eight-membered analogue 26 was also isolated.^{ix}

Although there is no universal scale of nucleofugicity for various leaving groups in S_{N}Ar,^{x} it is widely recognized that the nitro and fluoro groups frequently have similar reactivity. Accordingly, when benzimidazole 28 was subjected to the standard reaction conditions (Scheme 4), the fluorine was efficiently displaced with the formation of the same cyclic ether 11 previously obtained by nitro displacement in alcohol 10 (Table 1, entry 4).

In contrast, the chloro group in alcohol 29 (Scheme 5) proved significantly less reactive, and no product of its displacement was detected when the reaction was carried out under standard conditions (NaH, DMF, rt, 24 h). When an analogous reaction was performed at elevated temperature, a mixture of isomeric alkenes 30 was isolated as the main product, and only traces (˜2%) of the expected cyclic ether 5 were detected. Presumably, under forcing reaction conditions, the intermediate cyclic ether 5 undergoes base-catalyzed isomerization into alkenes 30.^{xi}

Intermolecular S_{N}Ar of the benzimidazole-activated nitro group has also been examined. As anticipated, this process is significantly less efficient than its intramolecular counterpart (Scheme 6).^{xii}

In conclusion, a synthetically useful and operationally simple method for the preparation of rotationally restricted 2-aryl-1H-benzimidazoles via intramolecular S_{N}Ar of the nitro group by alkoxides has been developed. The scope of this transformation should be subject to structural variation with respect to substituent diversity on both the two aromatic subunits and the nucleophile-bearing arm. These methodologies should also be extendable beyond O-nucleophiles, thus providing a novel entry into various heterocyclic systems.

The compounds and methods described herein are useful for, but not limited to treating, inhibiting, or delaying the onset of cancers. The compounds and methods are also useful in the treatment of precancers and other incidents of undesirable cell proliferation. According to the methods described herein, the compounds, or combinations, thereof are administered to a subject experiencing undesirable cell proliferation. The compounds and methods are useful for treating cancers including, but not limited to, leukemia, melanoma, non-small cell lung cancer, colon cancer, cancers of the central nervous system, ovarian cancer, breast cancer, kidney cancer, and prostate cancer. Furthermore, they are useful in the prevention of these cancers in individuals with precancers, as well as individuals prone to these disorders.

The compounds described herein are useful in treating, inhibiting, or delaying the onset of HIV-related illnesses. According to the methods described herein, the compounds, or combinations thereof, are administered to a subject infected with HIV. The method comprises contacting a cell A method of treating a subject infected with HIV comprising the step of administering a therapeutically effective amount of a compound of claim 1 to a subject in need of such treatment.

The method of claim 16 wherein the compound is 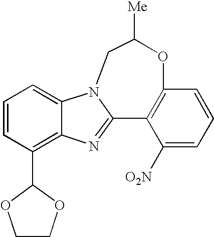
or a metabolite or prodrug thereof.

The terms “treatment,” “treating,” and “therapy” as used herein refer to curative therapy, prophylactic therapy, and preventative therapy. When the compounds described herein are used to treat unwanted proliferating cells, including cancers, “treatment” includes partial or total destruction of the undesirable proliferating cells with minimal destructive effects on normal cells. In practicing the method of treating a subject infected with HIV, “treatment” includes lessening, managing or delaying the onset of symptoms associated with HIV. In practicing the present method of treatment or use as an antibacterial agent, a pharmaceutical composition comprising a therapeutically effective amount of the compounds of described herein is applied to the site of infection in the host subject before or after the host subject is exposed to the bacterium.

The term “prevention” as used herein includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those at risk of developing precancers and cancers. Also encompasses is delaying the onset of symptoms associated with HIV in a subject. Also encompassed by this definition is the prevention of bacterial infections in subjects that have been exposed to or may be exposed to undesirable bacterial agents.

The terms “therapeutically effective” and “pharmacologically effective” are intended to qualify the amount of each agent which will achieve the goal of improvement in disease severity and the frequency of incidence. As used herein, the terms “therapeutically effective amount” and “pharmacologically effective amount” mean the total active amount of the compound that is sufficient to show a meaningful subject or patient benefit, i.e., a reduction in tumor size, arrest, inhibition of tumor growth and/or motility or metastasis, and/or an increase in apoptosis, and/or a reduction in the symptoms related to the presence of the tumor for cancers, or a lessening, or delaying of symptoms associated with HIV for HIV treatment. Or when the compounds are used as antibacterial agents, the term “therapeutically effective amount” means the total amount of the compound that is sufficient to show a meaningful benefit, i.e., treatment, healing, prevention, amelioration, or reduction in the symptoms of the bacterial infection or an increase in rate of healing, amelioration or reduction in the symptoms of such infection. Preferably the amount of the compound administered is from about 0.001 ng to about 1 mg/kg body weight. Initially, the attending physician may choose to administer low doses of the composition and gradually increase the dosage until the optimal therapeutic benefit is achieved.

The term “subject” for purposes of treatment includes any human or animal subject who has a disorder characterized by unwanted, rapid cell proliferation. Such disorders include, but are not limited to cancers and precancers. For methods of prevention the subject is any human or animal subject, and preferably is a human subject who is at risk of obtaining a disorder characterized by unwanted, rapid cell proliferation, such as cancer. The subject may be at risk due to exposure to carcinogenic agents, being genetically predisposed to disorders characterized by unwanted, rapid cell proliferation, and so on. For other methods described herein, “subjects” include subjects infected with HIV. “Subjects” may also include a human or animal subject that has or may come into contact with bacterial agents that may cause infections. Besides being useful for human treatment, the compounds described herein are also useful for veterinary treatment of mammals, including companion animals and farm animals, such as, but not limited to dogs, cats, horses, cows, sheep, and pigs. Preferably, subject means a human.

The agents described herein may be administered orally, intravenously, intranasally, rectally, or by any means which delivers an effective amount of the active agent to the tissue or site to be treated. It will be appreciated that different dosages may be required for treating different disorders. An effective amount of an agent is that amount which causes a statistically significant decrease in neoplastic cell count, growth, or size.

The dosage form and amount can be readily established by reference to known treatment or prophylactic regiments. The amount of therapeutically active compound that is administered and the dosage regimen for treating a disease condition with the compounds and/or compositions described herein depends on a variety of factors, including the age, weight, sex, and medical condition of the subject, the severity of the disease, the route and frequency of administration, the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The daily dose can be administered in one to four doses per day.

The active agents may be administered along with a pharmaceutical carrier and/or diluent. The agents may also be administered in combination with other agents, for example, in association with other chemotherapeutic or immunostimulating drugs or therapeutic agents. Examples of pharmaceutical carriers or diluents useful with the compounds described herein include any physiological buffered medium, i.e., about pH 7.0 to 7.4 comprising a suitable water soluble organic carrier. Suitable water soluble organic carriers include, but are not limited to corn oil, dimethylsulfoxide, gelatin capsules, etc.

When used as antibacterial agents, the compounds described herein may be incorporated into a topical composition. Preferably the topical composition comprises a solvent. A liquid carrier such as water, petroleum, oils of animal or plant origin such as peanut oil, mineral oil, soybean oil, or sesame oil, corn oil, or synthetic oils may be added. The liquid form of the pharmaceutical composition may further contain a physiological saline solution, dextrose or other saccharide solution, or glycols such as ethylene glycol, propylene glycol or polyethylene glycol. The preparation of such topical composition having suitable pH, isotonicity, and stability, is within the skill in the art.

The compounds described herein may also be useful in the the development of non-linear optic (NLO) devices. This involves coupling the homochiral tetracyclic units in specific polymers that can then be self-assembed into monolayers to fabricate optoelectronic devices. The tetracyclic products from the S_{N}Ar reaction may also find use in the the development of NLO devices. This will involve coupling the homochiral tetracyclic units in specific polymers that can then be self-assembed into monolayers to fabricate optoelectronic devices. For example, these polymers could be used to prepare an electro-optic modulator also known as a Mach-Zender interferometer (M-ZI), splits light and then modifies one of the streams of light so that the recombined light streams encode information from an external electrical input. In the modified stream, the light-wave is changed by applying an electric field, which changes the refractive index of the material. For the material to change in a predictable manner, its molecules have to have a net orientation (so the dipoles point in the same direction).

### Experimental Procedures

General Methods All reactions were performed under anhydrous conditions and an inert atmosphere of argon or nitrogen in the oven-dried glassware with magnetic stirring. Yields refer to chromatographically and spectroscopically (¹H NMR) homogenous materials, unless otherwise indicated. Reagents were used as obtained from commercial sources, or purified according to the guidelines of Perrin and Armarego.^{xiii} Evaporation in vacuo refers to the removal of volatiles on a Büchi rotory evaporator attached to an in-house vacuum system (˜20 mm Hg). Flash chromatography was carried out using Merck Kiesegel 60 F₂₅₄ (230-400 mesh) silica gel following the method of Still et al.^{xiv} Only distilled solvents were used as eluents. Thin-layer chromatography (TLC) was performed on Merck DC-Alufolien plates pre-coated with silica gel 60 F₂₅₄, that were visualized either by quenching of ultraviolet fluorescence, or by charring with 5% w/v phosphomolybdic acid in 95% EtOH, 10% w/v ammonium molybdate in 1M H₂SO₄, or 10% KMnO₄ in 1M H₂SO₄. Observed retention factors (R_{f}) are quoted to the nearest 0.05. All reaction solvents were distilled before use, and stored over activated 4 Å molecular sieves, unless otherwise indicated. Anhydrous CH₂Cl₂ was obtained by refluxing over CaH₂. Anhydrous THF was obtained by distillation, immediately before use, from sodium/benzophenone ketyl under an inert atmosphere of nitrogen. Anhydrous DMF was obtained by distillation under reduced pressure from CaH₂, and stored over 4 Å molecular sieves. Petroleum ether refers to the fraction of light petroleum boiling between 40 and 60° C. High-resolution mass spectrometry (HRMS) measurements are valid to ±5 ppm. Melting points (mp) are quoted to the nearest 0.5° C. Elemental analyses were performed by Atlantic Microlab, Inc., Norcross, Ga.

### Synthesis of 2-Aryl-1H-benzimidazoles

Each of the previously unreported 2-aryl-1H-benzimidazoles used in these studies was prepared from an appropriate benzoic acid and 1,2-diaminobenzene derivative. Thus, the benzoic acid was converted to the corresponding acyl chloride by treatment with oxalyl chloride in CH₂Cl₂ in the presence of a catalytic amount of DMF. The crude acyl chloride was used directly to acylate the 1,2-diaminbenzene derivative in CH₂Cl₂ in the presence of Et₃N. The crude N-monoacyl product was subsequently subjected to dehydrocyclization in boiling glacial AcOH in the presence of AcONa to give, after chromatographic purification, the required benzimidazole in high overall yield (>80% in each case). In regards to analytical characterization of these benzimidazoles, it should be mentioned that, due to NH-tautomerizm, it was frequently difficult to obtain good quality ¹³C NMR spectra for these compounds.

### 4-Methyl-2-(2-nitrophenyl)-1H-benzimidazole (37)

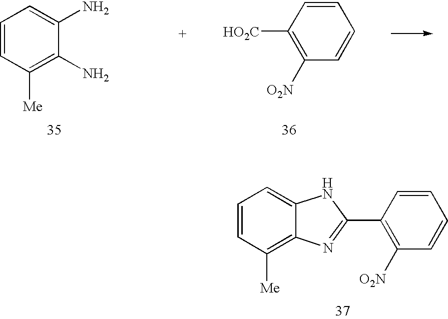

To a suspension of 2-nitrobenzoic acid 36 (30.0 g, 180 mmol) in CH₂Cl₂ (200 mL) added (COCl)₂ (20 mL, 0.23 mol), followed by two drops of DMF. The resulting mixture was stirred at rt for 17 h, and evaporated in vacuo to give a clear oil. The crude acyl chloride was dissolved in CH₂Cl₂ (100 mL), and added dropwise over 1 h into an ice-cooled solution of 2,3-diaminotoluene^{xv} 35 (21.3 g, 175 mmol) and Et₃N (33 mL, 0.23 mol) in CH₂Cl₂ (1.5 L). After an additional 3 h at 0° C.→rt, the volatiles were removed in vacuo, and the residue was refluxed in glacial AcOH (40 mL) in the presence of AcONa (14.7 g, 175 mmol) for 19 h. The reaction mixture was cooled to rt, evaporated in vacuo, and partitioned between CH₂Cl₂ and water. The biphasic mixture was cooled in an ice bath, and neutralized with solid K₂CO₃ during vigorous stirring. The phases were separated, and the extraction was completed with additional portions of CH₂Cl₂. The combined organic extracts were dried (MgSO₄), and evaporated in vacuo. Purification by flash chromatography (silica gel, CH₂Cl₂→CH₂Cl₂/EtOAc, 6/1) gave the title compound 37 (40.3 g, 91%) as a yellow solid: R_{f}=0.55 (CH₂Cl₂/EtOAc, 3/1); mp 167.0-167.5° C (EtOAc/petroleum ether); ¹H NMR (250 MHz, d₆-Me₂CO) δ 2.56 (s, 3H), 7.05 (d, J=7.0 Hz, 1H), 7.15 (t, J=7.5 Hz, 1H), 7.42 (br d, J=6.5 Hz, 1H), 7.67 (dt, J=7.5, 2.0 Hz, 1H), 7.73 (dt, J=7.5, 1.5 Hz, 1H), 7.90 (˜dd, J=7.5, 2.0 Hz, 1H), 7.95 (˜dd, J=7.5, 2.0 Hz, 1H), and 12.2 (br s, 1H); ¹³C{¹H} NMR (101 MHz, d₆-DMSO) δ 16.48, 17.00, 109.1, 116.6, 121.6, 122.0, 123.0, 123.5, 124.3, 124.7, 124.8, 128.8, 130.7, 131.2, 131.3, 132.5, 132.7, 134.3, 134.5, 143.2, 143.4, 146.7, 147.3, 148.9, and 149.0;^{xvi} IR (CHCl₃) νₘₐₓ 1533, 1449, and 1349 cm¹; MS (ESI) m/z (rel intensity) 254 (100%, MH⁺) and 208 (20); HRMS calcd for C₁₄H₁₂N₃O₂ (MH⁺) 254.0929, found 254.0925; Anal. Calcd for C₁₆H₁₃N₃O₂: C, 66.40; H, 4.38; N, 16.59. Found: C, 66.50; H, 4.37; N, 16.61.

### 2-(3-Chloro-2-nitrophenyl)-4-methyl-1H-benzimidazole (39)

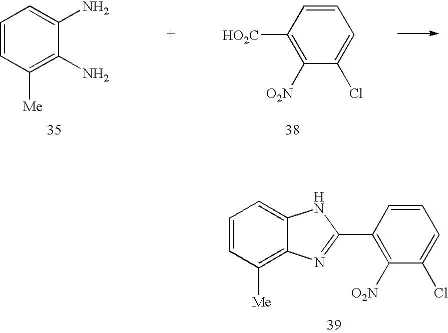

To a suspension of 3-chloro-2-nitrobenzoic acid 38 (5.00 g, 24.8 mmol) in CH₂Cl₂ (20 mL) was added (COCl)₂ (2.8 mL, 32 mmol), followed by a drop of DMF. After 1 h, the resulting clear solution was evaporated in vacuo to give a white solid. The crude acyl chloride was dissolved in CH₂Cl₂ (50 mL), and added dropwise over 30 min to a solution of 2,3-diaminotoluene^{xv} 35 (2.94 g, 24.1 mmol) and Et₃N (4.5 mL, 32 mmol) in CH₂Cl₂ (250 mL) at 0° C. After an additional 2 h at 0° C.→rt, the volatiles were removed in vacuo to give a yellow solid. The solid was dissolved in glacial AcOH (50 mL), AcONa (2.03 g, 24.8 mmol) was added, and the mixture was refluxed for 13 h. The reaction mixture was cooled to rt, evaporated in vacuo, and partitioned between CH₂Cl₂ and water. The biphasic mixture was cooled in an ice bath, and neutralized with solid K₂CO₃ during vigorous stirring. The phases were separated, and the extraction was completed with additional portions of CH₂Cl₂. The combined organic extracts were dried (MgSO₄), and evaporated in vacuo. Purification by flash chromatography (silica gel, CH₂Cl₂→CH₂Cl₂/EtOAc, 10/1) gave the title compound 39 (6.23 g, 91%) as a light yellow solid: R_{f}=0.70 (CH₂Cl₂/EtOAc, 9/1); mp 201.5-202.5° C. (Me₂CO); ¹H NMR (250 MHz, d₆-Me₂CO) δ 2.55 (s, 3H), 7.06 (d, J=7.0 Hz, 1H), 7.16 (t, J=7.5 Hz, 1H), 7.42 (d, J=7.5 Hz, 1H), 7.72 (t, J=8.0 Hz, 1H), 7.77 (dd, J=8.0, 2.0 Hz, 1H), 8.07 (dd, J=7.0, 2.0 Hz, 1H), and 12.0 (br s, 1H); IR (KBr) νₘₐₓ 1542, 1441, and 1373 cm⁻¹; MS (ESI) m/z (rel intensity) 288 (100%, MH⁺) and 242 (35); HRMS calcd for C₁₄H₁₁ClN₃O₂ (MH⁺) 288.0540, found 288.0522; Anal. Calcd for C₁₄H₁₀ClN₃O₂: C, 58.45; H, 3.50; Cl, 12.32; N, 14.61. Found: C, 58.40; H, 3.44; Cl, 12.43; N, 14.48.

### 4-Methyl-2-(3-methyl-2-nitrophenyl)-1H-benzimidazole (41)

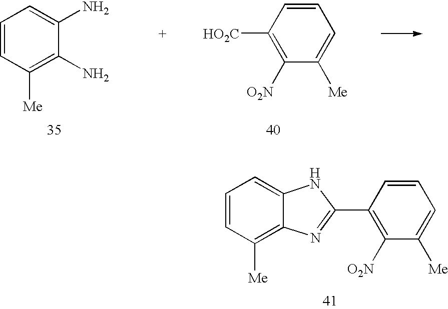

To a suspension of 3-methyl-2-nitrobenzoic acid 40 (5.00 g, 27.6 mmol) in CH₂Cl₂ (20 mL) was added (COCl)₂ (2.9 mL, 39 mmol), followed by a drop of DMF. After 3 h, the resulting clear solution was evaporated in vacuo to give a white solid. The crude acyl chloride was dissolved in CH₂Cl₂ (50 mL), and added dropwise over 30 min to a solution of 2,3-diaminotoluene^{xv} 35 (3.01 g, 24.6 mmol) and Et₃N (4.6 mL, 33 mmol) in CH₂Cl₂ (300 mL) at 0° C. After an additional 4 h at 0° C.→rt, the volatiles were removed in vacuo to give a yellow solid. The solid was dissolved in glacial AcOH (50 mL), AcONa (2.26 g, 27.6 mmol) was added, and the mixture was refluxed for 17 h. The reaction mixture was cooled to rt, evaporated in vacuo, and partitioned between CH₂Cl₂ and water. The biphasic mixture was cooled in an ice bath, and neutralized with solid K₂CO₃ during vigorous stirring. The phases were separated, and the extraction was completed with additional portions of CH₂Cl₂. The combined organic extracts were dried (MgSO₄), and evaporated in vacuo to give a crude product as an off-white solid. Purification by flash chromatography (silica gel, CH₂Cl₂→CH₂Cl₂/EtOAc, 30/1) gave the title compound 41 (5.85 g, 89%) as a white solid: R_{f}=0.45 (EtOAc/petroleum ether, 1/1); mp 186.0-187.5° C. (EtOAc/petroleum ether); ¹H NMR (400 MHz, d₆-DMSO) δ 2.34 (s, 3H), 2.54 (s, 3H), 7.04 (d, J=7.0 Hz, 1H), 7.13 (t, J=7.5 Hz, 1H), 7.42 (br s, 1H), 7.60 (d, J=7.5 Hz, 1H), 7.69 (t, J=7.5 Hz, 1H), and 7.94 (br s, 1H); IR (CHCl₃) νₘₐₓ 1537 and 1370 cm⁻¹; MS (ESI) m/z (rel intensity) 290 (70%, MNa⁺), 268 (100), and 222 (45); HRMS calcd for C₁₅H₁₃N₃NaO₂ (MNa⁺) 290.0905, found 290.0890; Anal. Calcd for C₁₅H₁₃N₃O₂: C, 67.40; H, 4.90; N, 15.72. Found: C, 67.27; H, 4.86; N, 15.73.

### 2-(2-Fluorophenyl)-4-methyl-1H-benzimidazole (43)

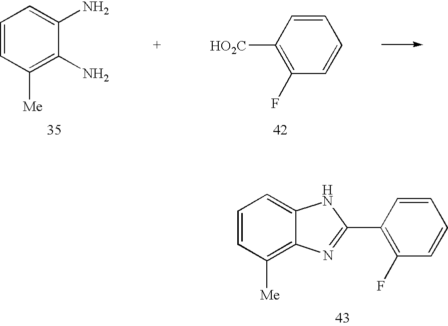

To a suspension of 2-fluorobenzoic acid 42 (5.00 g, 35.7 mmol) in CH₂C₁₂ (20 mL) was added (COCl)₂ (4.1 mL, 46 mmol), followed by a drop of DMF. The resulting mixture was stirred at rt for 16 h, and evaporated in vacuo to give a yellow oil. The crude acyl chloride was dissolved in CH₂Cl₂ (50 mL), and added dropwise over 1 h to a solution of 2,3-diaminotoluene^{xv} 35 (4.22 g, 34.6 mmol) and Et₃N (6.3 mL, 45 mmol) in CH₂Cl₂ (250 mL) at 0° C. After an additional 2 h at 0° C.→rt, the volatiles were removed in vacuo, and the residue was refluxed in glacial AcOH (50 mL) in the presence of AcONa (2.93 g, 35.7 mmol) for 8 h. The reaction mixture was cooled to rt, evaporated in vacuo, and partitioned between CH₂Cl₂ and water. The biphasic mixture was cooled in an ice bath, and neutralized with solid K₂CO₃ during vigorous stirring. The phases were separated, and the extraction was completed with additional portions of CH₂Cl₂. The combined organic extracts were dried (MgSO₄), and evaporated in vacuo to give an orange foam. Purification by flash chromatography (silica gel, CH₂Cl₂→CH₂Cl₂/EtOAc, 20/1) gave the title compound 43 (6.80 g, 87%) as a white solid: R_{f}=0.45 (EtOAc/petroleum ether, 3/1); mp 161.0-161.5° C. (EtOAc/petroleum ether); ¹H NMR (250 MHz, CDCl₃) δ 2.54 (s, 3H), 6.97 (d, J=7.0 Hz, 1H), 7.03-7.17 (m, 2H), 7.19 (d, J=7.5 Hz, 1H), 7.22-7.46 (m, 2H), 8.40 (dt, J=8.0, 2.0 Hz, 1H), and 9.78 (br s, 1H); IR (CHCl₃) νₘₐₓ 1466 cm⁻¹; MS (ESI) m/z (rel intensity) 227 (100%, MH⁺); HRMS calcd for C₁₄H₁₂FN₂ (MH⁺) 227.0984, found 227.0987; Anal. Calcd for C₁₄H₁₁FN₂: C, 74.32; H, 4.90; N, 12.38. Found: C, 74.06; H, 4.88; N, 12.36.

### 2-(2-Chlorophenyl)-4-methyl-1H-benzimidazole (45)

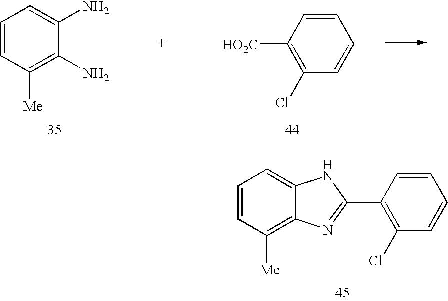

To a suspension of 2-chlorobenzoic acid 44 (5.00 g, 31.9 mmol) in CH₂Cl₂ (30 mL) was added (COCl)₂ (3.6 mL, 42 mmol), followed by a drop of DMF. The resulting mixture was stirred at rt for 90 min, and evaporated in vacuo to give a clear oil. The crude acyl chloride was dissolved in CH₂Cl₂ (50 mL), and added dropwise over 40 min to a solution of 2,3-diaminotoluene^{xv} 35 (3.78 g, 30.9 mmol) and Et₃N (5.8 mL, 42 mmol) in CH₂Cl₂ (250 mL) at 0° C. After an additional 3 h at 0° C.→rt, the volatiles were removed in vacuo to give a pale brown oil. The residue was refluxed in glacial AcOH (50 mL) in the presence of AcONa (2.62 g, 31.9 mmol) for 15 h. The reaction mixture was cooled to rt, evaporated in vacuo, and partitioned between CH₂Cl₂ and water. The biphasic mixture was cooled in an ice bath, and neutralized with solid K₂CO₃ during vigorous stirring. The phases were separated, and the extraction was completed with additional portions of CH₂Cl₂. The combined organic extracts were dried (MgSO₄), and evaporated in vacuo to give a yellow foam. Purification by flash chromatography (silica gel, petroleum ether→petroleum ether/EtOAc, 3/1) gave the title compound 45 (6.80 g, 88%) as a white foam: R_{f}=0.35 (petroleum ether/EtOAc, 3/1); ¹H NMR (250 MHz, CDCl₃) δ 2.54 (s, 3H), 6.98 (˜d, J=7.0 Hz, 1H), 7.08 (t, J=7.5 Hz, 1H), 7.19-7.26 (m, 2H), 7.29-7.38 (m, 2H), 8.11-8.15 (m, 1H), and 9.70 (br s, 1H); IR (CHCl₃) νₘₐₓ 1449, 1396, and 1045 cm⁻¹; MS (ESI) m/z (rel intensity) 265 (30%, MNa⁺) and 243(100); HRMS calcd for C₁₄H₁₁ClN₂Na (MNa⁺) 265.0508, found 265.0510.

### 2-(2-Methoxyphenyl)-1H-benzimidazole (48)

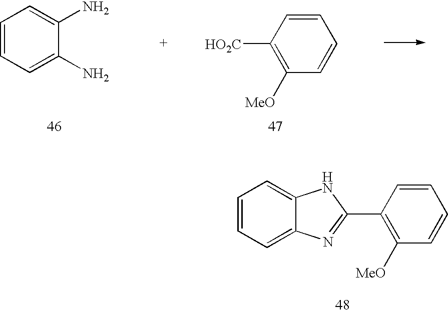

To a solution of 2-methoxybenzoic acid 47 (5.00 g, 32.9 mmol) in CH₂Cl₂ (30 mL) was added (COCl)₂ (3.7 mL, 43 mmol), followed by a drop of DMF. After 16 h at rt, the resulting clear solution was evaporated in vacuo to give a pale yellow oil. The crude acyl chloride was dissolved in CH₂Cl₂ (50 mL), and added dropwise over 30 min to an ice-cooled solution of 1,2-diaminobenzene 46 (3.89 g, 31.9 mmol) and Et₃N (6.0 mL, 43 mmol) in CH₂Cl₂ (250 mL) an additional 3 h at 0° C.→rt, the volatiles were removed in vacuo to give a brown solid. The solid was dissolved in glacial AcOH (50 mL), AcONa (2.7 g, 33 mmol) was added, and the mixture was refluxed for 19 h. The reaction mixture was cooled to rt, evaporated in vacuo, and partitioned between CH₂Cl₂ and water. The biphasic mixture was cooled in an ice bath, and neutralized with solid K₂CO₃ during vigorous stirring. The phases were separated, and the extraction was completed with additional portions of CH₂Cl₂. The combined organic extracts were dried (MgSO₄), and evaporated in vacuo to give a crude product as a yellow solid. Purification by flash chromatography (silica gel, CH₂Cl₂→CH₂Cl₂/EtOAc, 8/1) gave the title compound 48 (7.10 g, 93%) as a white solid: R_{f}=0.60 (CH₂Cl₂/EtOAc, 3/1); mp 179.5-180.5° C. (EtOAc/petroleum ether) (Lit.^{xvii} 181° C.); ¹H NMR (250 MHz, CDCl₃) δ 3.95 (s, 3H), 6.49 (d, J=8.5 Hz, 1H), 7.03 (dt, J=7.5, 1.0 Hz, 1H), 7.12-7.20 (m, 2H), 7.30 (ddd, J=8.5, 7.5, 2.0 Hz, 1H), 7.54 (br s, 2H), 8.49 (dd, J=8.0, 2.0 Hz, 1H), and 10.6 (br s, 1H); ¹³C{¹H} NMR (63 MHz, CDCl₃) δ 55.92, 111.5, 117.9, 121.7, 122.5, 130.2, 131.2, 149.9, and 156.8; IR (CHCl₃) νₘₐₓ 1472, 1441, 1279, and 1240 cm⁻¹; MS (ESI) m/z (rel intensity) 225 (100%, MH⁺); HRMS calcd for C₁₄H₁₃N₂O (MH⁺) 225.1028, found 225.1020.

### 2-(3-Chloro-2-nitrophenyl)-1H-benzimidazole (49)

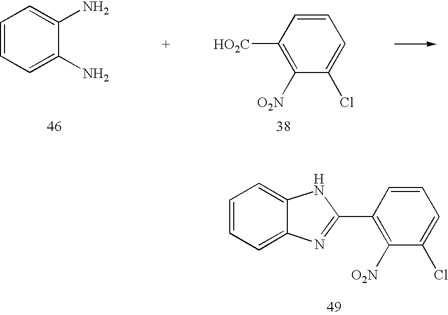

To a suspension of 3-chloro-2-nitrobenzoic acid 38 (5.00 g, 24.8 mmol) in CH₂Cl₂ (20 mL) was added (COCl)₂ (2.8 mL, 32 mmol), followed by a drop of DMF. After 1 h, the resulting clear solution was evaporated in vacuo to give a white solid. The crude acyl chloride was dissolved in CH₂Cl₂ (50 mL), and added dropwise over 1 h to a solution of 1,2-diaminobenzene 46 (2.60 g, 24.1 mmol) and Et₃N (4.5 mL, 32 mmol) in CH₂Cl₂ (250 mL) at 0° C. After an additional 2 h at 0° C.→rt, the volatiles were removed in vacuo to give a pale yellow solid. The solid was dissolved in glacial AcOH (50 mL), AcONa (2.03 g, 24.8 mmol) was added, and the mixture was refluxed for 19 h. The reaction mixture was cooled to rt, evaporated in vacuo, and partitioned between CH₂Cl₂ and water. The biphasic mixture was cooled in an ice bath, and neutralized with solid K₂CO₃ during vigorous stirring. The phases were separated, and the extraction was completed with additional portions of CH₂Cl₂. The combined organic extracts were dried (MgSO₄), and evaporated in vacuo to give a crude product as a creamy solid. Purification by flash chromatography (silica gel, CH₂Cl₂→CH₂Cl₂/EtOAc, 2/1) gave the title compound 49 (5.78 g, 88%) as an off-white solid: R_{f}=0.70 (EtOAc); mp>260° C. (Me₂CO); ¹H NMR (400 MHz, d₆-DMSO) δ 7.22-7.30 (m, 2H), 7.58-7.67 (m, 2H), 7.81 (t, J=8.0 Hz, 1H), 7.88 (d, J=8.0 Hz, 1H), 8.13 (d, J=7.5 Hz, 1H), and 13.3 (br s, 1H); IR (KBr) νₘₐₓ 1541, 1450, 1440, 1415, and 1376 cm⁻¹; MS (ESI) m/z (rel intensity) 274 (100%, MH⁺); HRMS calcd for C₁₃H₉ClN₃O₂ (MH⁺) 274.0383, found 274.0372; Anal. Calcd for C₁₃H₈ClN₃O₂: C, 57.05; H, 2.95; Cl, 12.95; N, 15.35. Found: C, 56.85; H, 2.86; Cl, 13.13; N, 15.27.

### Epoxide-Ring Opening with 2-Aryl-1H-benzimidazoles

The free NH group of 2-aryl-1H-benzimidazoles, and in particular 2-(2-nitrophenyl)-1H-benzimidazoles, is poorly nucleophilic, so its reaction with epoxides is not very facile. The Cu(OTf)₂-catalyzed reaction used in these studies to prepare the desired alcohols does not usually go to completion. With a few notable exceptions, the levels of conversions are usually low (<50%). In general, 4-substituted 2-aryl-1H-benzimidazoles are far better substrates than their unsubstituted counterparts. It presumably stems from a significantly reduced ability, due to steric reasons, to form Cu(II) complexes by the substrates (and the products derived from them) that are heavily substituted in the vicinity of the benzimidazole nitrogen. For the unsubstituted benzimidazoles, in turn, the catalytic cycle ceases to operate relatively quickly, as an active Cu(II) species gets converted into various unreactive complexes. On a few occasions, we attempted to use stochiometric amounts of Cu(OTf)₂, but it did not lead to improved yields of the alcohols. As far as epoxide substrates are concerned, it was found that only terminal epoxides are reactive enough, with the 2-monosubstitued derivatives being significantly better substrates than the 2,2-disubstitued ones.

### 1-[4-Methyl-2-(2-nitrophenyl)benzimidazol-1-yl]propan-2-ol (4)

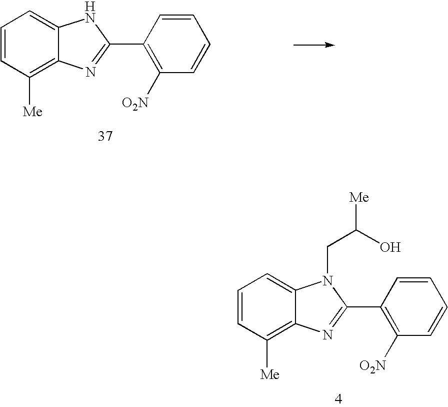

To a solution of benzimidazole 37 (1.27 g, 5.00 mmol) in MeCN (8 mL) were added propylene oxide (2.9 g, 50 mmol) and Cu(OTf)₂ (362 mg, 1.00 mmol). After 22 h at 40° C., the reaction mixture was cooled to rt and evaporated in vacuo. The residue was partitioned between CH₂Cl₂ and satd NaHCO₃, and stirred at rt for 30 min. The phases were separated, and the extraction was completed with additional portions of CH₂Cl₂. The combined organic extracts were dried (MgSO₄), and evaporated in vacuo to give a brown oil. Purification by flash chromatography (silica gel, CH₂Cl₂→CH₂Cl₂/EtOAc, 4/1) gave the recovered starting material 37 (670 mg, 53%) and the title compound 4 (499 mg, 32%) as a pale yellow solid. Alcohol 4: R_{f}=0.55 (EtOAc); mp 171.0-172.0° C. (EtOAc/petroleum ether); ¹H NMR (400 MHz, CDCl₃) δ 1.13 (d, J=6.0 Hz, 3H), 2.66 (s, 4H), 3.95 (dd, J=14.5, 8.5 Hz, 1H), 4.03 (dd, J=14.5, 3,5 Hz, 1H), 4.10-4.20 (m, 1H), 7.15 (d, J=6.5 Hz, 1H), 7.23-7.34 (m, 2H), 7.64 (dd, J=7.0, 1.5 Hz, 1H), 7.68 (dd, J=8.0, 1.5 Hz, 1H), 7.73 (dt, J=7.5, 1.0 Hz, 1H), and 8.16 (dd, J=8.0, 1.0 Hz, 1H); ¹³C{¹H} NMR (101 MHz, CDCl₃) δ 16.65, 20.63, 52.03, 65.98, 107.8, 123.0, 123.2, 124.9, 126.3, 130.1, 130.9, 132.9, 133.2, 134.6, 142.2, 148.6, and 149.0; IR (CHCl₃) νₘₐₓ 1534, 1458, 1397, and 1348 cm⁻¹; MS (ESI) m/z (rel intensity) 312 (100%, MH⁺); HRMS calcd for C₁₇H₁₈N₃O₃ (MH⁺) 312.1348, found 312.1328; Anal. Calcd for C₁₇H₁₇N₃O₃: C, 65.58; H, 5.50; N, 13.50. Found: C, 65.49; H, 5.45; N, 13.39.

### 1-[4-Methyl-2-(2-nitrophenyl)benzimidazol-1-yl]hexan-2-ol (6)

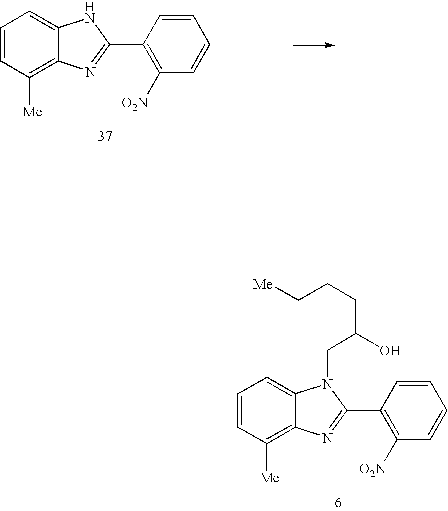

To a solution of benzimidazole 37 (1.27 g, 5.00 mmol) in MeCN (8 mL) were added 1,2-epoxyhexane (1.5 g, 15 mmol) and Cu(OTf)₂ (362 mg, 1.00 mmol). After 20 h at reflux, the reaction mixture was cooled to rt and evaporated in vacuo. The residue was partitioned between CH₂Cl₂ and satd NaHCO₃, and stirred at rt for 30 min. The phases were separated, and the extraction was completed with additional portions of CH₂Cl₂. The combined organic extracts were dried (MgSO₄), and evaporated in vacuo to give a brown oil. Purification by flash chromatography (silica gel, CH₂Cl₂→CH₂Cl₂/EtOAc, 4/1) gave the recovered starting material 37 (329 mg, 26%) and the title compound 6 (754 mg, 43%) as a yellow solid. Alcohol 6: R_{f}=0.65 (EtOAc); ¹H NMR (400 MHz, CDCl₃) δ 0.87 (t, J=6.5 Hz, 3H), 1.15-1.49 (m, 6H), 2.66 (s, 3H), 3.87-3.96 (m, 2H), 4.07 (dd, J=18.0, 7.0 Hz, 1H), 7.15 (d, J=6.5 Hz, 1H), 7.23-7.33 (m, 2H), 7.60-7.73 (m, 3H), and 8.15 (d, J=7.5 Hz, 1H); ¹³C{¹H} NMR (63 MHz, CDCl₃) δ 14.32, 17.16, 22.89, 27.89, 34.81, 51.49, 70.38, 108.3, 123.5, 123.7, 125.3, 126.7, 130.5, 131.4, 133.5, 133.7, 135.1, 142.6, 149.2, and 149.4; IR (CHCl₃) νₘₐₓ 2960, 1535, 1458, 1397, 1348, and 1242 cm⁻¹; MS (ESI) m/z (rel intensity) 354 (100%, MH⁺) and 254 (15); HRMS calcd for C₂₀H₂₄N₃O₃ (MH⁺) 354.1817, found 354.1830.

### 3,3-Dimethyl-1-[2-(2-nitrophenyl)benzimidazol-1-yl]butan-2-ol (8)

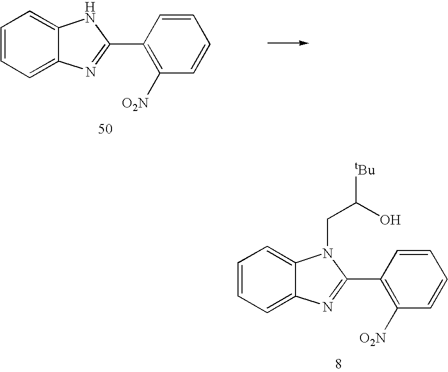

To a suspension of benzimidazole 50^{xviii} (2.39 g, 10.0 mmol) in MeCN (40 mL) were added 3,3-dimethyl-1,2-epoxybutane (5.0 g, 50 mmol) and Cu(OTf)₂ (724 mg, 2.00 mmol). After 17 h at reflux, the reaction mixture was cooled to rt and evaporated in vacuo. The residue was partitioned between CH₂Cl₂ and satd NaHCO₃, and stirred at rt for 30 min. The phases were separated, and the extraction was completed with additional portions of CH₂Cl₂. The combined organic extracts were dried (MgSO₄), and evaporated in vacuo to give a brown foam. Purification by flash chromatography (silica gel, CH₂Cl₂→CH₂Cl₂/EtOAc, 4/1) gave the title compound 8 (603 mg, 18%) as a white solid: R_{f}=0.70 (EtOAc); mp 191.5-193.0° C. (EtOAc/petroleum ether); 1H NMR (400 MHz, CDCl₃) δ 0.90 (s, 9H), 2.58 (br s, 1H), 3.61 (d, J=10.0 Hz, 1H), 3.96 (dd, J=14.5, 10.0 Hz, 1H), 4.24 (d, J=14.5 Hz, 1H), 7.32 (t, J=6.5 Hz, 1H), 7.36 (t, J=7.0 Hz, 1H), 7.62-7.74 (m, 4H), and 8.15 (d, J=8.0 Hz, 1H); ¹³C{¹H } NMR (63 MHz, CDCl₃) δ 25.86, 34.72, 47.56, 77.97, 110.9, 120.5, 122.9, 123.6, 125.1, 126.6, 131.3, 133.4, 133.5, 135.5, 143.4, 149.5, and 150.1; IR (CHCl₃) νₘₐₓ 2965, 1534, 1458, 1404, and 1349 cm⁻¹; MS (ESI) m/z (rel intensity) 340 (100%, MH⁺) and 240 (10); HRMS calcd for C₁₉H₂₁N₃NaO₃ (MNa⁺) 362.1481, found 362.1473; Anal. Calcd for C₁₉H₂₁N₃O₃: C, 67.24; H, 6.24; N, 12.38. Found: C, 67.14; H, 6.25; N, 12.31.

### 3,3-Dimethyl-1-[4-methyl-2-(2-nitrophenyl)benzimidazol-1-yl]butan-2-ol (10)

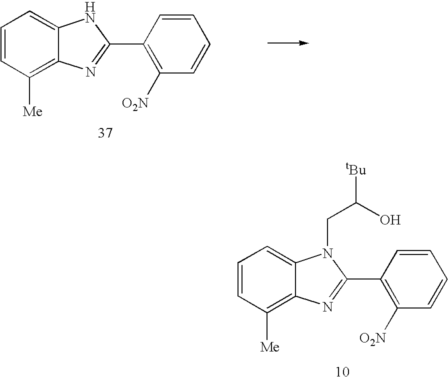

To a solution of benzimidazole 37 (2.00 g, 7.91 mmol) and 3,3-dimethyl-1,2-epoxy-butane (3.95 g, 39.5 mmol) in MeCN (40 mL) was added Cu(OTf)₂ (573 mg, 1.58 mmol). The reaction mixture was refluxed for 24 h, cooled to rt, and evaporated in vacuo. The residue was partitioned between CH₂Cl₂ and satd NaHCO₃, and stirred at rt for 30 min. The phases were separated, and the extraction was completed with additional portions of CH₂Cl₂. The combined organic extracts were dried (MgSO₄), and evaporated in vacuo to give a brown solid. Purification by flash chromatography (silica gel, CH₂Cl₂→CH₂Cl₂/EtOAc, 2/1) gave the recovered starting material 37 (780 mg, 39%) and the title compound 10 (1.39 g, 50%) as a pale yellow solid. Alcohol 10: R_{f}=0.70 (EtOAc); mp 203.5-204.5° C. (EtOAc/petroleum ether); ¹H NMR (250 MHz, CDCl₃) δ 0.83 (s, 9H), 2.01 (d, J=4.0 Hz, 1H), 2.61 (s, 3H), 3.56 (dd, J=10.0, 4.0 Hz, 1H), 3.87 (dd, J=14.5, 10.0 Hz, 1H), 4.18 (d, J=14.5 Hz, 1H), 7.07 (d, J=7.0 Hz, 1H), 7.19 (t, J=8.0 Hz, 1H), 7.26 (d, J=8.0 Hz, 1H), 7.57-7.72 (m, 3H), and 8.10 (˜dd, J=8.0, 1.0 Hz, 1H); ¹³C{¹H} NMR (101 MHz, CDCl₃) δ 16.69, 25.41, 34.22, 47.17, 77.60, 107.8, 122.9, 123.1, 124.8, 126.5, 130.2, 130.8, 133.0, 133.1, 134.7, 142.4, 148.8, and 149.2; IR (CHCl₃) νₘₐₓ 2964, 1534, 1458, 1398, and 1348 cm⁻¹; MS (ESI) m/z (rel intensity) 354 (100%, MH⁺) and 254 (10); HRMS calcd for C₂₀H₂₃N₃NaO₃ (MNa⁺) 376.1637, found 376.1644; Anal. Calcd for C₂₀H₂₃N₃O₃: C, 67.97; H, 6.56; N, 11.89. Found: C, 67.97; H, 6.61; N, 11.91.

### 2-Methyl-1-[4-methyl-2-(2-nitrophenyl)benzimidazol-1-yl]propan-2-ol (14)

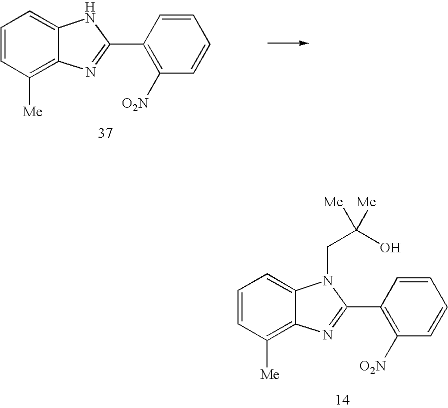

To a solution of benzimidazole 37 (1.27 g, 5.0 mmol) and 1,2-epoxy-2-methylpropane (1.8 g, 25 mmol) in MeCN (8 mL) was added Cu(OTf)₂ (362 mg, 1.00 mmol) to give a dark-brown suspension. The reaction mixture was refluxed for 24 h, cooled to rt, and evaporated in vacuo. The residue was partitioned between CH₂Cl₂ and satd NaHCO₃, and stirred at rt for 1 h. The phases were separated, and the extraction was completed with additional portions of CH₂Cl₂. The combined organic extracts were dried (MgSO₄), and evaporated in vacuo to give a brown oil. Purification by flash chromatography (silica gel, CH₂Cl₂→CH₂Cl₂/EtOAc, 10/1; re-purification: silica gel, CH₂Cl₂/EtOAc, 2/1) gave the recovered starting material 37 (1.08 g, 85%) and the title compound 14 (197 mg, 12%) as a pale yellow solid: R_{f}=0.55 (EtOAc); ¹H NMR (400 MHz, CDCl₃) δ 1.12 (s, 6H), 1.98 (s, 1H), 2.64 (s, 3H), 4.08 (s, 2H), 7.09 (d, J=7.5 Hz, 1H), 7.21 (t, J=7.5 Hz, 1H), 7.31 (d, J=9.0 Hz, 1H), 7.61 (dd, J=7.5, 1.0 Hz, 1H), 7.64 (t, J=7.5 Hz, 1H), 7.64 (dt, J=8.0, 1.5 Hz, 1H), 7.72 (dt, J=7.5, 1.5 Hz, 1H), and 8.12 (dd, J=8.0, 1.0 Hz, 1H); ¹³C{¹H} NMR (101 MHz, CDCl₃) δ 16.67, 27.69, 55.07, 71.99, 108.7, 122.9, 123.2, 125.0, 127.1, 130.2, 130.7, 132.8, 133.1, 135.7, 142.3, 148.8, and 149.1; IR (CHCl₃) νₘₐₓ 1533, 1456, and 1348 cm₋₁; MS (ESI) m/z (rel intensity) 326 (100%, MH⁺), 308 (20), and 254 (70); HRMS calcd for C₁₈H₁₉N₃NaO₃ (MNa⁺) 348.1324, found 348.1318.

### 1-[2-(3-Chloro-2-nitrophenyl)-4-methylbenzimidazol-1-yl]-3,3-dimethylbutan-2-ol (16)

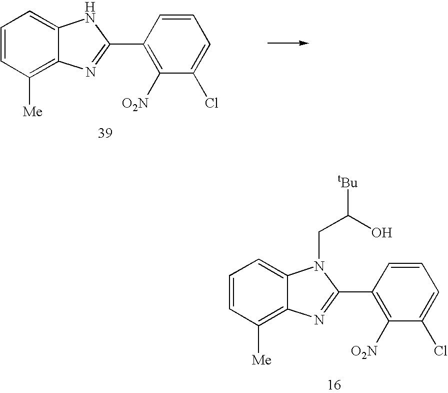

To a solution of benzimidazole 39 (2.00 g, 7.04 mmol) and 3,3-dimethyl-1,2-epoxybutane (3.5 g, 35 mmol) in MeCN (50 mL) was added Cu(OTf)₂ (510 mg, 1.41 mmol). The reaction mixture was refluxed for 22 h, cooled to rt, and evaporated in vacuo. The residue was partitioned between CH₂Cl₂ and satd NaHCO₃, and stirred at rt for 30 min. The phases were separated, and the extraction was completed with additional portions of CH₂Cl₂. The combined organic extracts were dried (MgSO₄), and evaporated in vacuo to give a brown oil. Purification by flash chromatography (silica gel, CH₂Cl₂→EtOAc) gave the recovered starting material 39 (1.31 g, 66%) and the title compound 16 (554 mg, 20%) as a white solid. Alcohol 16: R_{f}=0.70 (CH₂Cl₂/EtOAc, 2/1); mp 235.5-237.0° C. (EtOAc); ¹H NMR (250 MHz, CDCl₃) δ 0.94 (s, 9H), 1.97 (d, J=4.5 Hz, 1H), 2.63 (s, 3H), 3.65 (ddd, J=10.0, 4.5, 1.5 Hz, 1H), 4.05 (dd, J=14.5, 10.0 Hz, 1H), 4.33 (dd, J=14.5, 1.5 Hz, 1H), 7.05-7.28 (m, 3H), 7.53 (t, J=7.5 Hz, 1H), 7.64 (d, J=8.0 Hz, 1H), and 7.69 (d, J=7.0 Hz, 1H); ¹³C{¹H} NMR (101 MHz, d₆-DMSO) δ 16.14, 25.48, 34.41, 46.76, 75.40, 109.1, 122.3, 123.0, 124.6, 125.5, 129.0, 131.3, 131.8, 132.0, 134.9, 141.9, 146.3, and 148.8; IR (CHCl₃) νₘₐₓ 2965, 1546, 1457, and 1365 cm⁻¹; MS (ESI) m/z (rel intensity) 388 (100%, MH⁺) and 288 (10); HRMS calcd for C₂₀H₂₂ClN₃NaO₃ (MNa⁺) 410.1247, found 410.1234.

### 3,3-Dimethyl-1-[4-methyl-2-(3-methyl-2-nitrophenyl)benzimidazol-1-yl]butan-2-ol (18)

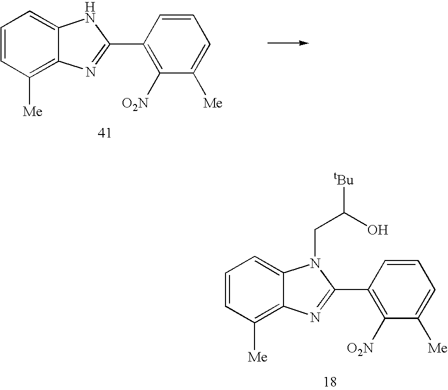

To a solution of benzimidazole 41 (2.00 g, 7.5 mmol) in MeCN (15 mL) were added 3,3-dimethyl-1,2-epoxybutane (3.0 g, 30 mmol) and Cu(OTf)₂ (543 mg, 1.5 mmol) to give a dark-brown solution. After 19 h at reflux, the reaction mixture was cooled to rt and evaporated in vacuo. The residue was partitioned between CH₂Cl₂ and satd NaHCO₃, and stirred at rt for 30 min. The phases were separated, and the extraction was completed with additional portions of CH₂Cl₂. The combined organic extracts were dried (MgSO₄), and evaporated in vacuo to give a brown foam. Purification by flash chromatography (silica gel, CH₂Cl₂→CH₂Cl₂/EtOAc, 4/1) gave the recovered starting material 41 (247 mg, 12%) and the title compound 18 (1.08 g, 39%) as a white solid. Alcohol 18: R_{f}=0.55 (CH₂Cl₂/EtOAc, 3/1); mp 216.0-217.5° C. (EtOAc/petroleum ether); ¹H NMR (400 MHz, d₆-DMSO) δ 0.86 (s, 9H), 2.40 (s, 3H), 2.51 (s, 3H), 3.59 (dd, J=10.5, 5.5 Hz, 1H), 3.95 (dd, J=14.0, 10.5 Hz, 1H), 4.27 (d, J=14.0 Hz, 1H), 5.12 (d, J=5.5 Hz, 1H), 7.06 (d, J=7.0 Hz, 1H), 7.21 (t, J=7.5 Hz, 1H), 7.44 (d, J=8.0 Hz, 1H), 7.69 (dd, J=8.0, 1.0 Hz, 1H), 7.71 (t, J=7.5 Hz, 1H), and 7.99 (dd, J=7.0, 1.0 Hz, 1H); ¹³C{¹H} NMR (101 MHz, d₆-DMSO) δ 16.18, 17.28, 25.47, 34.37, 46.73, 75.61, 109.1, 122.0, 122.5, 124.0, 128.7, 130.1, 130.4 (2×C), 133.0, 134.9, 141.9, 147.9, and 150.8; IR (CHCl₃) νₘₐₓ 2964, 1535, 1457, and 1365 cm₋₁; MS (ESI) m/z (rel intensity) 390 (75%, MNa⁺), 368 (100), and 268 (5); HRMS calcd for C₂₁H₂₅N₃NaO₃ (MNa⁺) 390.1794, found 390.1805; Anal. Calcd for C₂₁H₂₅N₃O₃: C, 68.64; H, 6.86; N, 11.44. Found: C, 68.64; H, 6.86; N, 11.30.

### 1-[2-(2-Fluorophenyl)-4-methylbenzimidazol-1-yl]-3,3-dimethylbutan-2-ol (28)

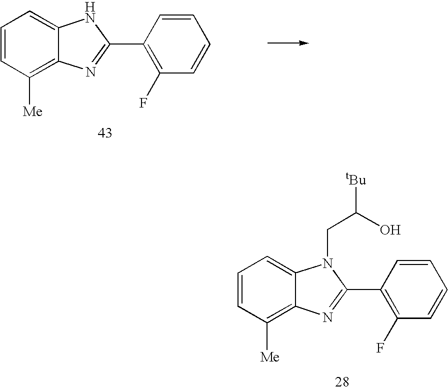

To a solution of benzimidazole 43 (2.00 g, 8.85 mmol) and 3,3-dimethyl-1,2-epoxybutane (4.4 g, 44 mmol) in MeCN (50 mL) was added Cu(OTf)₂ (641 mg, 1.77 mmol). The reaction mixture was refluxed for 16 h, cooled to rt, and evaporated in vacuo. The residue was partitioned between CH₂Cl₂ and satd NaHCO₃, and stirred at rt for 30 min. The phases were separated, and the extraction was completed with additional portions of CH₂Cl₂. The combined organic extracts were dried (MgSO₄), and evaporated in vacuo to give a brown oil. Purification by flash chromatography (silica gel, CH₂Cl₂→EtOAc) gave the recovered starting material 43 (1.40 g, 70%) and the title compound 28 (298 mg, 10%) as a white solid. Alcohol 28: R_{f}=0.70 (EtOAc/CH₂Cl₂, 1/1); mp 178.5-179.0° C. (EtOAc/petroleum ether); ¹H NMR (400 MHz, CDCl₃) δ 0.89 (s, 9H), 1.76 (s, 1H), 2.74 (s, 3H), 3.49 (d, J=10.0 Hz, 1H), 4.06 (dd, J=14.5, 10.0 Hz, 1H), 4.31 (d, J=14.5 Hz, 1H), 7.15 (d, J=7.0 Hz, 1H), 7.19-7.33 (m, 3H), 7.37 (d, J=8.0 Hz, 1H), 7.52 (dd, J=13.0, 7.0 Hz, 1H), and 7.67 (t, J=7.0 Hz, 1H); ¹³C{¹H} NMR (101 MHz, CDCl₃) δ 16.76, 25.29, 34.23, [46.6 (d, J=3.0 Hz)?], 78.31, 108.0, 115.8 (d, J=21.5 Hz), 119.2 (d, J=14.5 Hz), 122.7, 122.9, 124.5 (d, J=3.5 Hz), 130.1, 131.9 (d, J=8.0 Hz), 132.7 (d, J=2.0 Hz), 135.1, 142.3, 148.3, and 160.0 (d, J=249 Hz); IR (CHCl₃) νₘₐₓ 2964, 1642, 1459, and 1391 cm⁻¹; MS (ESI) m/z (rel intensity) 327 (100%, MH⁺) and 227 (10); HRMS calcd for C₂₀H₂₃FN₂NaO (MNa⁺) 349.1692, found 349.1698; Anal. Calcd for C₂₀H₂₃FN₂O: C, 73.59; H, 7.10; N, 8.58. Found: C, 73.50; H, 7.31; N, 8.64.

### 1-[2-(2-Chlorophenyl)-4-methylbenzimidazol-1-yl]propan-2-ol (29)

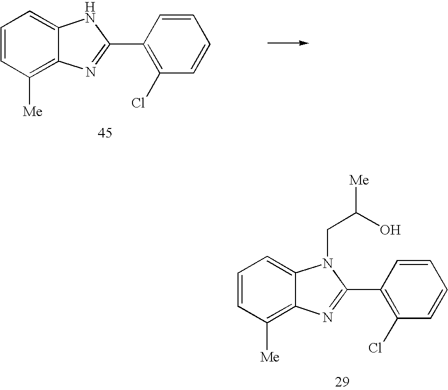

To a solution of benzimidazole 45 (3.00 g, 12.3 mmol) in MeCN (15 mL) were added propylene oxide (3.6 g, 62 mmol) and Cu(OTf)₂ (891 mg, 2.46 mmol) to give a brown suspension. After 20 h at 65° C., the reaction mixture was cooled to rt and evaporated in vacuo. The residue was partitioned between CH₂Cl₂ and satd NaHCO₃, and stirred at rt for 40 min. The phases were separated, and the extraction was completed with additional portions of CH₂Cl₂. The combined organic extracts were dried (MgSO₄), and evaporated in vacuo to give a light brown foam. Purification by flash chromatography (silica gel, CH₂Cl₂→EtOAc) gave the title compound 29 (851 mg, 23%) as a white solid: R_{f}=0.35 (CH₂Cl₂/EtOAc, 3/1); mp 164.0-165.0° C. (EtOAc/petroleum ether); ¹H NMR (400 MHz, CDCl₃) δ 1.02 (d, J=6.0 Hz, 3H), 2.49 (br s, 1H), 2.70 (s, 3H), 3.99 (d, J=6.0 Hz, 2H), 4.06 (heptet, J=6.0, 6.0 Hz, 1H), 7.13 (d, J=7.5 Hz, 1H), 7.24 (t, J=7.0 Hz, 1H), 7.30 (d, J=8.0 Hz, 1H), 7.36 (dt, J=7.5, 1.0 Hz, 1H), 7.44 (dt, J=7.5, 1.5 Hz, 1H), 7.50 (dd, J=8.0, 1.0 Hz, 1H), and 7.53 (dd, J=7.5, 1.5 Hz, 1H); ¹³C{¹H} NMR (101 MHz, CDCl₃) δ 16.76, 20.78, 51.63, 66.20, 108.0, 122.9 (2×C), 126.9, 129.6, 130.1, 130.2, 131.2, 132.7, 134.2, 134.6, 142.1, and 150.4; IR (CHCl₃) νₘₐₓ 1607, 1453, 1391, 1335, and 1241 cm⁻¹; MS (ESI) m/z (rel intensity) 323 (35%, MNa⁺), 301 (100), and 243 (45); HRMS calcd for C₁₇H₁₈ClN₂O (MNa⁺) 301.1107, found 301.1112; Anal. Calcd for C₁₇H₁₇ClN₂O: C, 67.88; H, 5.70; Cl, 11.79; N, 9.31. Found: C, 68.02; H, 5.65; Cl, 11.98; N, 9.30.

### Synthesis of Diols (20) and (24)

The diols used in these studies were prepared by the Sharpless asymmetric dihydroxylation reaction (AD) of appropriate N-allyl substituted 2-aryl-1H-benzimidazoles with commercially available AD-mix-α[containing the (DHQ)₂PHAL ligand]. ¹H NMR analysis of the corresponding Mosher esters revealed that the resulting diols were, in each case, virtually racemic. Presumably, the benzimidazole substrates can act as ligands for osmium and, thus, interfere with the, otherwise highly enantioselective, catalytic cycle.

### 1-Allyl-2-(2-nitrophenyl)-1H-benzimidazole (51)

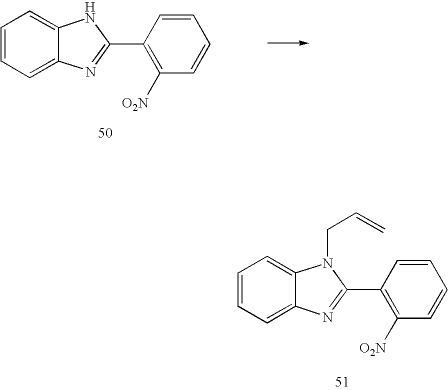

To an ice-cooled suspension of benzimidazole 50^{xvii} (3.35 g, 14.0 mmol) in THF (40 mL) was added NaH (60% w/w, 616 mg, 15.4 mmol) portionwise over 5 min. After 10 min at 0° C., the resulting red solution was treated with allyl bromide (1.6 mL, 18 mmol), and stirred at 0° C.→rt for 16 h. The reaction mixture was quenched with water, evaporated in vacuo, and partitioned between CH₂Cl₂ and water. The phases were separated, and the extraction was completed with additional portions of CH₂Cl₂. The combined organic extracts were dried (MgSO₄), and evaporated in vacuo to give a yellow oil. Purification by flash chromatography (silica gel, CH₂Cl₂→CH₂Cl₂/EtOAc, 2/1) gave the title compound 51 (3.80 g, 97%) as a pale yellow solid: R_{f}=0.55 (CH₂Cl₂/EtOAc, 3/1); mp 81.5-83.0° C. (EtOAc/petroleum ether); 1H NMR (400 MHz, CDCl₃) δ 4.64 (s, 2H), 5.07 (˜d, J=17.0 Hz, 1H), 5.21 (d, J=10.5 Hz, 1H), 5.84-5.94 (m, 1H), 7.28-7.47 (m, 3H), 7.62 (d, J=7.0 Hz, 1H), 7.65-7.88 (m, 3H), and 8.19 (d, J=8.0 Hz, 1H); ¹³C{¹H} NMR (63 MHz, CDCl₃) δ 46.98, 110.4, 117.9, 120.0, 122.4, 123.1, 124.7, 125.9, 131.0, 131.6, 132.5, 133.1, 134.8, 143.0, 148.7, and 149.3; IR (CHCl₃) νₘₐₓ 1535, 1459, 1401, and 1348 cm⁻¹; MS (ESI) m/z (rel intensity) 280 (100%, MH⁺); HRMS calcd for C₁₆H₁₄N₃O₂ (MH⁺) 280.1086, found 280.1092; Anal. Calcd for C₁₆H₁₃N₃O₂: C, 68.81; H, 4.69; N, 15.05. Found: C, 68.74; H, 4.71; N, 15.13.

### 3-[2-(2-Nitrophenyl)benzimidazol-1-yl]propane-1,2-diol (20)

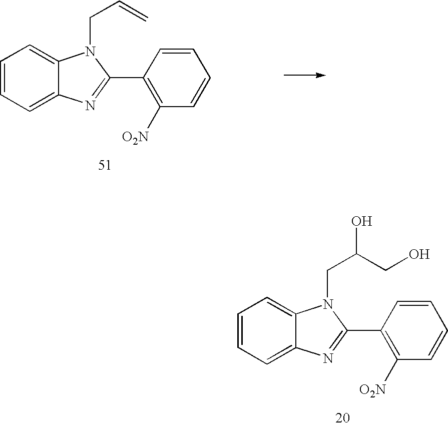

A solution of AD-mix-β (6.2 g) in ^{t}BuOH (25 mL) and H₂O (25 mL) was stirred at rt for 1 h. The mixture was cooled in an ice bath, and benzimidazole 51 (1.15 g, 4.12 mmol) was added. After 13 h at 0° C.→rt, Na₂SO₃ (4.5 g, 36 mmol) was added, and the reaction mixture was stirred at rt for 1 h. The resulting gray solution was diluted with CH₂Cl₂ and H₂O, and stirred for 5 min. The phases were separated, and the extraction was completed with additional portions of CH₂Cl₂. The combined organic extracts were dried (MgSO₄), and evaporated in vacuo to give a yellow solid. Purification by flash chromatography (silica gel, EtOAc/Me₂CO, 10/1) gave the title compound 20 (950 mg, 74%) as a yellow solid: R_{f}=0.15 (EtOAc); mp 181.5-183.0° C. (EtOAc); [α]_{D}=−0.5 (c 0.61 in MeOH); ¹H NMR (400 MHz, d₆-DMSO) δ 3.27-3.40 (m, 2H), 3.84-3.90 (m, 1H), 3.96 (dd, J=14.5, 8.5 Hz, 1H), 4.29 (dd, J=14.5, 3.0 Hz, 1H), 4.76 (t, J=5.5 Hz, 1H), 5.11 (d, J=5.0 Hz, 1H), 7.26 (˜t, J=7.0 Hz, 1H), 7.33 (dt, J=7.0, 1.0 Hz, 1H), 7.66 (d, J=8.0 Hz, 1H), 7.71 (d, J=8.0 Hz, 1H), 7.83 (dt, J=7.5, 1.5 Hz, 1H), 7.90 (dt, J=7.5, 1.0 Hz, 1H), 7.97 (dd, J=7.5, 1.5 Hz, 1H), and 8.21 (dd, J=8.0, 1.0 Hz, 1H); ¹³C{¹H} NMR (63 MHz, d₆-DMSO) δ 47.78, 63.47, 70.03, 111.5, 119.1, 121.9, 122.5, 124.5, 125.4, 131.2, 133.0, 133.3, 135.4, 142.6, 149.1, and 149.5; IR (KBr) νₘₐₓ 3073, 1523, 1464, 1446, 1417, and 1349 cm⁻¹; MS (ESI) m/z (rel intensity) 314 (100%, MH⁺); HRMS calcd for C₁₆H₁₆N₃O₄ (MH⁺) 314.1141, found 314.1146; Anal. Calcd for C₁₆H₁₅N₃O₄: C, 61.34; H, 4.83; N, 13.41. Found: C, 61.46; H, 4.84; N, 13.50.

### (2R,2′R)-3,3,3-Trifluoro-2-methoxy-2-phenylpropionic acid 2-[2-(2-nitrophenyl)benzoimidazol-1-yl]-1-(3,3,3-trifluoro-2-methoxy-2-phenylpropionyloxymethyl)ethyl ester (52)

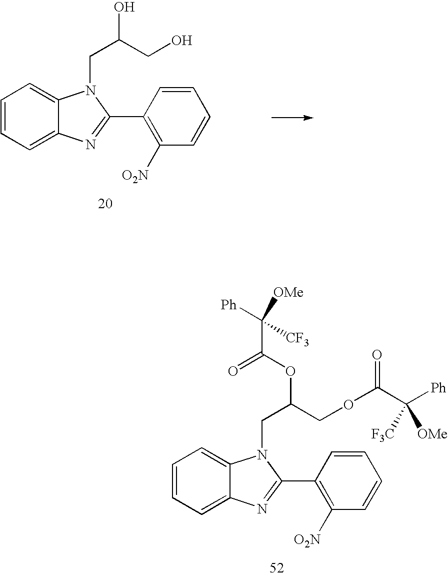

To an ice-cooled solution of diol 20 (20.0 mg, 64 μmol) in CH₂Cl₂ (2 mL) were added (R)-MTPA (59.8 mg, 0.26 mmol), DMAP (33 mg, 0.27 mmol), and DCC (158 mg, 0.77 mmol). The reaction mixture was stirred at 0° C.→rt for 40 h, and the resulting white suspension filtered through a plug of cotton. The filtrate was diluted with EtOAc, and washed successively with satd NaHCO₃, water, and brine. The organic layer was dried (MgSO₄), and evaporated in vacuo to give a pale yellow oil. Purification by flash chromatography (silica gel, CH₂Cl₂→CH₂Cl₂/EtOAc, 10/1) gave the title compound 52 (44 mg, 92%) as a pale yellow oil: R_{f}=0.25 (petroleum ether/EtOAc, 2/1); ¹H NMR (400 MHz, CDCl₃) δ 2.92 (s, 3H), 3.23 (s, 6H), 3.29 (s, 3H), 4.05-4.21 (m, 6H), 4.57 (dd, J=13.0, 3.0 Hz, 1H), 4.75 (dd, J=12.5, 2.5 Hz, 1H), 5.49-5.59 (m, 2H), 6.95-7.34 (m, 28H), 7.50-7.77 (m, 6H), and 8.02-8.13 (m, 2H); IR (CHCl₃) νₘₐₓ 1758 and 1534 cm⁻¹; MS (ESI) m/z (rel intensity) 768 (90%, MNa⁺) and 746 (100); HRMS calcd for C₃₆H₂₉F₆N₃NaO₈ (MNa⁺) 768.1757, found 768.1743.

### 1-Allyl-2-(3-chloro-2-nitrophenyl)-4-methylbenzimidazole (53) and 1-allyl-2-(3-chloro-2-nitrophenyl)-7-methylbenzimidazole (54)

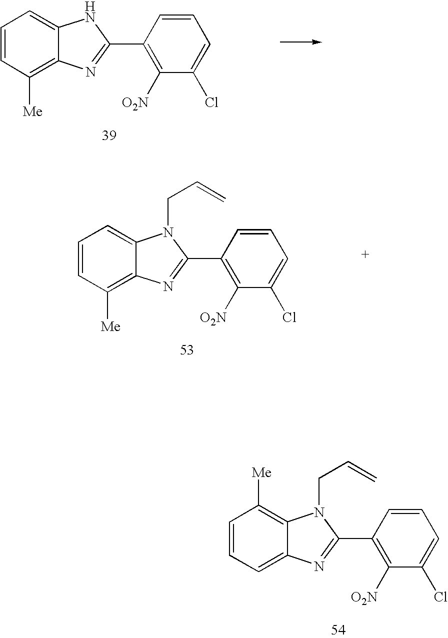

To a yellow solution of benzimidazole 39 (3.00 g, 10.5 mmol) in THF (25 mL) was added NaH (60% w/w, 462 mg, 11.6 mmol) portionwise at 0° C. After 15 min at rt, the resulting deep-orange solution was treated with allyl bromide (1.2 mL, 13.6 mmol), and stirred at rt for 18 h. The reaction mixture was quenched with water, evaporated in vacuo, and partitioned between CH₂Cl₂ and water. The phases were separated, and the extraction was completed with additional portions of CH₂Cl₂. The combined organic extracts were dried (MgSO₄), and evaporated in vacuo to give a yellow solid. Repetitive purification by flash chromatography (silica gel, CH₂Cl₂→CH₂Cl₂/EtOAc, 20/1) gave the title compound 53 (2.39 g, 70%) as a pale yellow solid, and the title compound 54 (618 mg, 18%) as a yellow solid.^{xix} Benzimidazole 53: R_{f}=0.55 (EtOAc/petroleum ether, 1/1); mp 124.5-125.5° C. (EtOAc/petroleum ether); ¹H NMR (400 MHz, CDCl₃) δ2.68 (s, 3H), 4.72-4.73 (m, 2H), 5.05 (d, J=17.0 Hz, 1H), 5.27 (d, J=10.0 Hz, 1H), 5.95 (ddt, J=17.0, 10.0, 3.5 Hz, 1H), 7.13 (d, J=6.0 Hz, 1H), 7.21-7.28 (m, 2H), 7.52-7.58 (m, 2H), and 7.67 (˜d, J=7.5 Hz, 1H); ¹³C{¹H} NMR (101 MHz, CDCl₃) δ 16.51, 47.10, 108.0, 117.8, 123.2, 123.7, 125.9, 126.5, 129.4, 130.7 (2×C), 131.8, 132.1, 134.7, 142.4, 145.8, and 149.5; IR (CHCl₃) νₘₐₓ 1546, 1455, and 1364 cm⁻¹; MS (ESI) m/z (rel intensity) 350 (100%, MNa⁺) and 328 (50); HRMS calcd for C₁₇H₁₄ClN₃NaO₂ (MNa⁺) 350.0672, found 350.0670; Anal. Calcd for C₁₃H₁₄ClN₃O₂: C, 62.30; H, 4.31; Cl, 10.82; N, 12.82. Found: C, 62.46; H, 4.29; Cl, 10.99; N, 12.86. Benzimidazole 54: R_{f}=0.50 (EtOAc/petroleum ether, 1/1); mp 144.0-145.0° C. (EtOAc/petroleum ether); 1H NMR (400 MHz, CDCl₃) δ 2.64 (s, 3H), 4.75 (˜dd, J=17.0, 2.0 Hz, 1H), 4.84-4.86 (m, 2H), 5.22 (˜dd, J=10.5, 1.5 Hz, 1H), 5.98 (ddt, J=17.0, 10.5, 4.0 Hz, 1H), 7.03 (d, J=7.5 Hz, 1H), 7.16 (t, J=7.5 Hz, 1H), 7.47-7.52 (m, 2H), and 7.60-7.66 (m, 2H); ¹³C{¹H} NMR (101 MHz, CDCl₃) δ 17.96, 47.82, 116.7, 118.6, 121.5, 122.9, 125.9, 126.3, 129.3, 130.7, 132.2, 133.7, 134.2, 143.4, 147.4, and 149.5 (one C atom obscured); IR (CHCl₃) νₘₐₓ 1545, 1454, 1394, and 1363 cm⁻¹; MS (ESI) m/z (rel intensity) 350 (100%, MNa⁺) and 328 (65); HRMS calcd for C₁₇H₁₅ClN₃O₂ (MH⁺) 328.0853, found 328.0859; Anal. Calcd for C₁₇H₁₄ClN₃O₂: C, 62.30; H, 4.31; Cl, 10.82; N, 12.82. Found: C, 62.52; H, 4.30; Cl, 10.89; N, 12.90.

### 3-[2-(3-Chloro-2-nitrophenyl)-4-methyl-benzimidazol-1-yl]propane-1,2-diol (24)

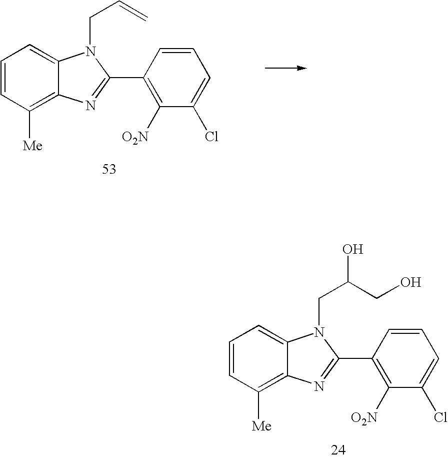

A solution of AD-mix-β (5.5 g) in ^{t}BuOH (24 mL) and H₂O (24 mL) was stirred at rt for 45 min. The mixture was cooled to 0° C., and benzimidazole 53 (1.07 g, 3.28 mmol) was added. After 70 h at 0° C.→rt, Na₂SO₃ (4.0 g, 32 mmol) was added, and the reaction mixture was stirred at rt for 1 h. The resulting gray suspension was diluted with CH₂Cl₂ and H₂O, and stirred for 5 min. The phases were separated, and the extraction was completed with additional portions of CH₂Cl₂. The combined organic extracts were dried (MgSO₄), and evaporated in vacuo to give a white foam. Purification by flash chromatography (silica gel, CH₂Cl₂→EtOAc) gave the recovered starting material 53 (116 mg, 11%) and the title compound 24 (1.01 g, 85%) as a yellow solid: R_{f}=0.50 (EtOAc); mp 165.5-166.5° C. (EtOAc/petroleum ether); ¹H NMR (400 MHz, d₆-DMSO) δ 2.50 (s, 3H), 3.29-3.41 (m, 2H), 3.82-3.94 (m, 1H), 4.04 (dd, J=14.5, 9.0 Hz, 1H), 4.35 (dd, J=14.5, 3.0 Hz, 1H), 4.76 (t, J=5.5 Hz, 1H), 5.14 (d, J=5.0 Hz, 1H), 7.08 (d, J=7.5 Hz, 1H), 7.23 (t, J=7.5 Hz, 1H), 7.50 (d, J=8.0 Hz, 1H), 7.82 (t, J=8.0 Hz. 1H), 7.98 (d, J=8.0 Hz, 1H), and 8.11 (d, J=7.5 Hz, 1H); ¹³C{¹H} NMR (101 MHz, d₆-DMSO) δ 16.12, 47.85, 63.47, 69.82, 109.1, 122.4, 123.0, 124.6, 125.5, 129.0, 131.0, 132.0 (2×C), 135.0, 141.8, 146.2, and 148.7; IR (KBr) νₘₐₓ 1539, 1459, 1439, and 1364 cm₋₁; MS (ESI) m/z (rel intensity) 384 (85%, MNa⁺) and 362 (100); HRMS calcd for C₁₇H₁₆ClN₃NaO₄ (MNa⁺) 384.0727 found 384.0744; Anal. Calcd for C₁₇H₁₆ClN₃O₄: C, 56.44; H, 4.46; Cl, 9.80; N, 11.61. Found: C, 56.58; H, 4.41; Cl, 9.79; N, 11.65.

### (2R,2′R)-3,3,3-Trifluoro-2-methoxy-2-phenylpropionic acid 2-[2-(3-chloro-2-nitrophenyl)-4-methylbenzoimidazol-1-yl]-1-(3,3,3-trifluoro-2-methoxy-2-phenylpropionyloxymethyl)-ethyl ester (55)

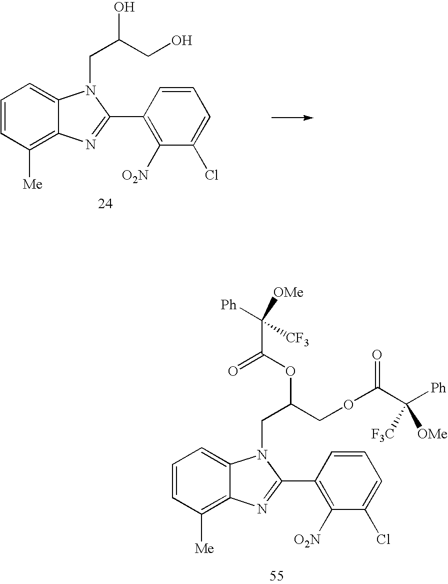

To an ice-cooled solution of diol 24 (15.0 mg, 42 μmol) in CH₂Cl₂ (3 mL) were added (R)-MTPA (69 mg, 0.30 mmol), DMAP (38 mg, 0.31 mmol), and DCC (182 mg, 0.88 mmol). The reaction mixture was stirred at 0° C.→rt for 48 h, and the resulting white suspension filtered through a plug of cotton. The filtrate was diluted with EtOAc, and washed successively with satd NaHCO₃, water, and brine. The organic layer was dried (MgSO₄), and evaporated in vacuo to give a pale yellow oil. Purification by flash chromatography (silica gel, CH₂Cl₂→CH₂Cl₂/EtOAc, 10/1) gave the title compound 55 (29 mg, 88%) as a clear oil: R_{f}=0.25 (petroleum ether/EtOAc, 3/1); ¹H NMR (400 MHz, CDCl₃) δ 2.55 (s, 3H), 2.57 (s, 3H), 2.91 (s, 3H), 3.28 (s, 6H), 3.31 (s, 3H), 4.08-4.27 (m, 6H), 4.57 (dd, J=13.0, 3.0 Hz, 1H), 4.75 (dd, J=13.0, 3.0 Hz, 1H), 5.56-5.62 (m, 2H), 7.02-7.39 (m, 30H), and 7.52-7.59 (m, 2H); IR (CHCl₃) νₘₐₓ 1759, 1545, 1453, 1269, and 1231 cm⁻¹; MS (ESI) m/z (rel intensity) 816 (90%, MNa⁺) and 794 (100); HRMS calcd for C₃₇H₃₀ClF₆N₃NaO₈ (MNa⁺) 816.1523, found 816.1529.

### 1-Allyl-2-(3-chloro-2-nitrophenyl)-1H-benzimidazole (56)

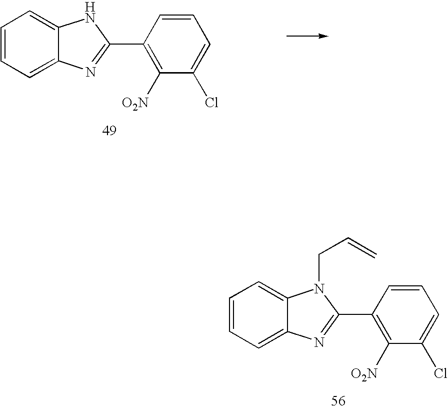

To a suspension of benzimidazole 49 (2.00 g, 7.3 mmol) in THF (25 mL) was added NaH (60% w/w, 321 mg, 8.0 mmol) portionwise at 0° C. After 15 min at rt, the resulting red-brown solution was treated with allyl bromide (821 μL, 8 mmol), and stirred at rt for 20 h. The reaction mixture was quenched with water, evaporated in vacuo, and partitioned between CH₂Cl₂ and water. The phases were separated, and the extraction was completed with additional portions of CH₂Cl₂. The combined organic extracts were dried (MgSO₄), and evaporated in vacuo to give a yellow solid. Purification by flash chromatography (silica gel, CH₂Cl₂→CH₂Cl₂/EtOAc, 20/1) gave the title compound 56 (2.24 g, 98%) as a white solid: R_{f}=0.45 (EtOAc/petroleum ether, 1/1); mp 148.5-149.5° C. (EtOAc/petroleum ether); ¹H NMR (400 MHz, CDCl₃) δ 4.69-4.71 (m, 2H), 5.01 (d, J=17.0 Hz, 1H), 5.23 (d, J=10.5 Hz, 1H), 5.92 (ddt, J=17.0, 10.5, 5.0 Hz, 1H), 7.24-7.37 (m, 3H), 7.48 (dd, J=7.5, 1.5 Hz, 1H), 7.52 (t, J=7.5 Hz, 1H), 7.64 (dd, J=8.0, 1.5 Hz, 1H), and 7.75-7.79 (m, 1H); ¹³C{¹H} NMR (101 MHz, CDCl₃) δ 47.04, 110.6, 117.9, 120.6, 122.9, 123.7, 125.6, 126.5, 129.2, 130.8, 131.7, 132.2, 135.1, 142.9, 146.7, and 149.4; IR (CHCl₃) νₘₐₓ 1546, 1458, 1441, 1393, and 1363 cm⁻¹; MS (ESI) m/z (rel intensity) 314 (100%, MH⁺); HRMS calcd for C₁₆H₁₃ClN₃O₂ (MH⁺) 314.0696, found 314.0709; Anal. Calcd for C₁₆H₁₂ClN₃O₂: C, 61.25; H, 3.86; Cl, 11.30; N, 13.39. Found: C, 61.31; H, 3.89; Cl, 11.38; N, 13.35.

### 3-[2-(3-Chloro-2-nitrophenyl)benzimidazol-1-yl]propane-1,2-diol (57)

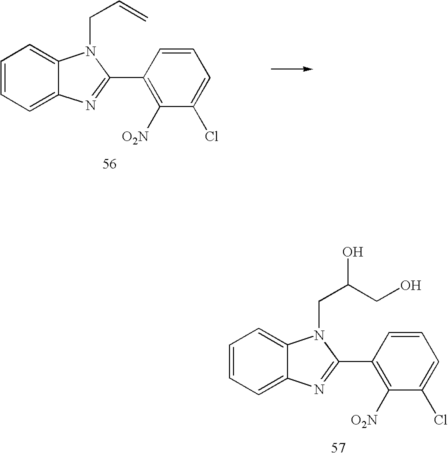

A solution of AD-mix-β (7.2 g) in ^{t}BuOH (29 mL) and H₂O (29 mL) was stirred at rt for 1 h. The reaction mixture was cooled in an ice bath, and benzimidazole 56 (1.50 g, 4.78 mmol) was added. After 45 h at 0° C.→rt, Na₂SO₃ (5.0 g, 40 mmol) was added, and the reaction mixture was stirred at rt for 1 h. The resulting gray suspension was diluted with CH₂Cl₂ and H₂O, and stirred for 5 min. The phases were separated, and the extraction was completed with additional portions of CH₂Cl₂. The combined organic extracts were dried (MgSO₄), and evaporated in vacuo to give an off-white solid. Purification by flash chromatography (silica gel, EtOAc→EtOAc/MeOH, 50/1) gave the recovered starting material 56 (869 mg, 58%) and the title compound 57 (527 mg, 32%). Diol 57: a white solid: R_{f}=0.20 (EtOAc); mp 187.5-189.0° C. (EtOAc); [α]_{D}=+0.8 (c 0.62 in MeOH); ¹H NMR (400 MHz, d₆-DMSO) δ3.29-3.42 (m, 2H), 3.86-3.95 (m, 1H), 4.09 (dd, J=14.5, 9.0 Hz, 1H), 4.39 (dd, J=14.5, 3.0 Hz, 1H), 4.79 (t, J=5.5 Hz, 1H), 5.18 (d, J=5.0 Hz, 1H), 7.27 (t, J=8.0 Hz, 1H), 7.34 (t, J=7.0 Hz, 1H), 7.68 (d, J=8.5 Hz, 1H), 7.70 (d, J=9.0 Hz, 1H), 7.82 (t, J=8.0 Hz, 1H), 7.97 (d, J=8.0 Hz, 1H), and 8.14 (d, J=8.0 Hz, 1H); ¹³C{¹H} NMR (101 MHz, d₆-DMSO) δ 47.78, 63.49, 69.88, 111.7, 119.5, 122.3, 123.1, 124.7, 125.2, 131.0, 132.0, 132.1, 135.5, 142.4, 147.0, and 148.7; IR (KBr) νₘₐₓ 3061, 1528, 1462, 1401, and 1358 cm⁻¹; MS (ESI) m/z (rel intensity) 348 (100%, MH⁺); HRMS calcd for C₁₆H₁₅ClN₃O₄ (MH⁺) 348.0751, found 348.0764; Anal. Calcd for C₁₆H₁₄ClN₃O₄: C, 55.26; H, 4.06; Cl, 10.19; N, 12.08. Found: C, 55.28; H, 4.09; Cl, 10.44; N, 12.01.

### Intramolecular S_{N}Ar Reactions with Monoalcohols

### (S)-(+)-6-tert-Butyl-11-[1,3]dioxolan-2-yl-6,7-dihydro-5-oxa-7a,12-diazadibenzo[a,e]azulene (3)

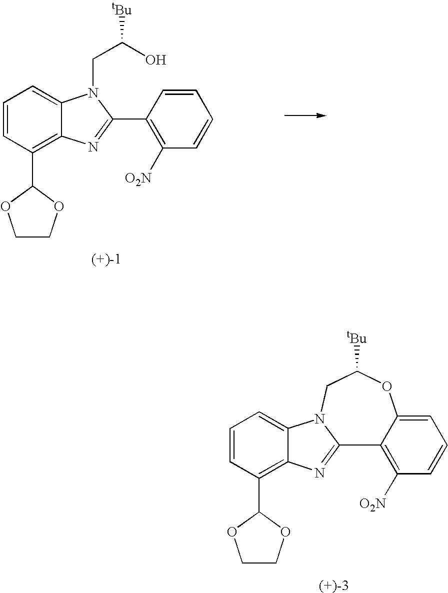 Method 1 (attempted etherification with 1,8-dibromooctane):

To a solution of alcohol (+)-1^{xx} (98% ee, 188 mg, 0.46 mmol) in anhydrous DMF (2 mL) was added NaH (60% w/w, 20 mg, 0.5 mmol), and the reaction mixture was stirred at rt for 50 min. 1,8-dibromooctane (42 μL, 0.23 mmol) was added, and the mixture was stirred at rt for 21 h. The reaction mixture was quenched with water, and diluted with EtOAc. The organic phase was washed repeatedly with water, dried (MgSO₄), and evaporated in vacuo to give an off-white solid. Purification by flash chromatography (silica gel, CH₂Cl₂→CH₂Cl₂/EtOAc, 10/1) gave the title compound (+)-3 (159 mg, 95%) as a white solid. CSP HPLC analysis (FIG. S1) revealed it to be of 98% optical purity.

Method 2:

To a solution of alcohol (+)-1^{xx} (98% ee, 337 mg, 0.82 mmol) in anhydrous DMF (3 mL) was added NaH (60% w/w, 36 mg, 0.9 mmol). After 15 h at rt, the reaction mixture was quenched with water, and diluted with EtOAc. The organic phase was washed repeatedly with water, dried (MgSO₄), and evaporated in vacuo to give an off-white solid. Purification by flash chromatography (silica gel, CH₂Cl₂→CH₂Cl₂/EtOAc, 10/1) gave the title compound (+)-3 (246 mg, 83%) as a white solid: mp 150.0-152.0° C. (EtOAc/petroleum ether); [α]_{D}=+147 (c 0.90 in CHCl₃).

### 6,11-Dimethyl-6,7-dihydro-5-oxa-7a,12-diaza-dibenzo[a,e]azulene (5)

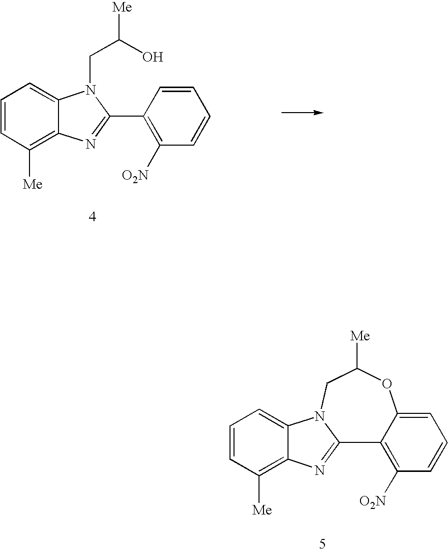

To a solution of alcohol 4 (142 mg, 0.46 mmol) in anhydrous DMF (1 mL) was added NaH (60% w/w, 20 mg, 0.50 mmol). After 14 h at rt, the reaction mixture was quenched with water, and diluted with EtOAc. The organic phase was washed repeatedly with water, dried (MgSO₄), and evaporated in vacuo to give an off-white solid. Purification by flash chromatography (silica gel, CH₂Cl₂→CH₂Cl₂/EtOAc, 10/1) gave the title compound 5 (94 mg, 78%) as a white solid: R_{f}=0.35 (petroleum ether/EtOAc, 9/1); ¹H NMR (250 MHz, CDCl₃) δ 1.44 (d, J=6.5 Hz, 3H), 2.65 (s, 3H), 4.05 (dd, J=14.0, 8.5 Hz, 1H), 4.22 (dd, J=14.0, 2.0 Hz, 1H), 4.43 (ddq, J=8.5, 6.5, 2.0 Hz, 1H), 6.94-7.14 (m, 5H), 7.24 (ddd, J=8.0, 7.5, 2.0 Hz, 1H), and 8.59 (dd, J=8.0, 2.0 Hz, 1H); ¹³C{¹H} NMR (101 MHz, CDCl₃) δ 19.07, 21.80, 53.85, 78.41, 108.8, 122.4, 123.6, 125.0, 125.3, 125.6, 132.3, 133.5, 133.7, 138.3, 144.6, 151.9, and 158.1; IR (CHCl₃) νₘₐₓ 1607, 1576, 1473, 1449, 1385, and 1233 cm⁻¹; MS (ESI) m/z (rel intensity) 265 (100%, MH⁺); HRMS calcd for C₁₇H₁₆N₂NaO (MNa⁺) 287.1160, found 287.1186.

### 6-Butyl-11-methyl-6,7-dihydro-5-oxa-7a,12-diazadibenzo[a,e]azulene (7)

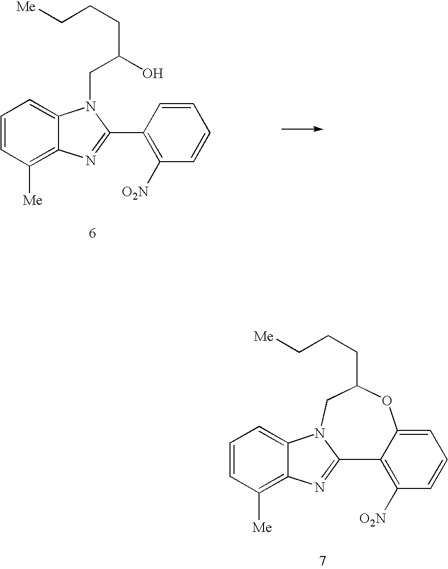

To a solution of alcohol 6 (664 mg, 1.88 mmol) in anhydrous DMF (62 mL) was added NaH (60% w/w, 83 mg, 2.1 mmol) to give a black solution. After 17 h at rt, the reaction mixture was quenched with water, and diluted with EtOAc. The organic phase was washed repeatedly with water, dried (MgSO₄), and evaporated in vacuo to give an off-white solid. Purification by flash chromatography (silica gel, CH₂Cl₂→CH₂Cl₂/EtOAc, 10/1) gave the title compound 7 (432 mg, 75%) as a white solid: R_{f}=0.45 (petroleum ether/EtOAc, 9/1); ¹H NMR (250 MHz, CDCl₃) δ 0.88 (t, J=7.0 Hz, 3H), 1.20-1.91 (m, 6H), 2.67 (s, 3H), 4.03 (dd, J=14.0, 9.0 Hz 1H), 4.22 (dd, J=14.0, 2.0 Hz, 1H), 6.95-7.14 (m, 5H), 7.24 (ddd, J=8.0, 7.0, 2.0 Hz, 1H), and 8.63 (dd, J=8.0, 2.0 Hz, 1H); ¹³C{¹H} NMR (63 MHz, CDCl₃) δ 14.47, 17.15, 22.92, 28.17 33.50, 51.21, 80.16, 107.0, 120.7, 121.4, 123.0, 123.3, 123.6, 130.3, 131.5, 131.8, 136.5, 142.7, 149.8, and 156.6; IR (CHCl₃) νₘₐₓ 1605, 1576, 1427, 1447, 1376, and 1321 cm⁻¹; MS (ESI) m/z (rel intensity) 307 (100%, MH⁺); HRMS calcd for C₂₀H₂₃N₂O (MH⁺) 307.1810, found 307.1798.

### 6-tert-Butyl-6,7-dihydro-5-oxa-7a,1 2-diazadibenzo[a,e]azulene (9)

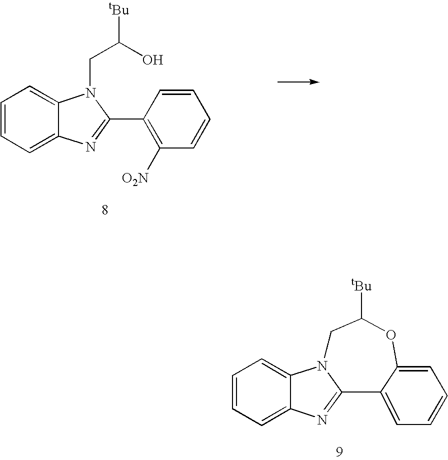

To a solution of alcohol 8 (100 mg, 0.29 mmol) in anhydrous DMF (2 mL) was added NaH (60% w/w, 12.8 mg, 0.32 mmol) to give a dark-green solution. After 11 h at rt, the reaction mixture was quenched with water, and diluted with EtOAc. The organic phase was washed repeatedly with water, dried (MgSO₄), and evaporated in vacuo to give a yellow solid. Purification by flash chromatography (silica gel, CH₂Cl₂→CH₂Cl₂/EtOAc, 10/1) gave the title compound 9 (74 mg, 86%) as a white solid: R_{f}=0.65 (CH₂Cl₂/EtOAc, 9/1); mp 149.0-150.0° C. (EtOAc/petroleum ether); ¹H NMR (400 MHz, CDCl₃) δ 1.09 (s, 9H), 3.83 (dd, J=9.5, 1.0 Hz, 1H), 4.19 (dd, J=13.5, 9.5 Hz, 1H), 4.49 (dd, J=13.5, 1.0 Hz, 1H), 7.01 (˜d, J=8.0 Hz, 1H), 7.09 (dt, J=8.0, 0.5 Hz, 1H), 7.13-7.29 (m, 4H), 7.74 (dd, J=6.5, 2.0 Hz, 1H), and 8.62 (dd, J=8.0, 1.5 Hz, 1H); ¹³C{¹H} NMR (101 MHz, CDCl₃) δ 26.13, 34.94, 47.54, 86.96, 109.1, 119.5, 119.7, 120.4, 122.6, 122.7, 123.0, 131.3 (2×C), 136.5, 142.8, 150.1, and 157.4; IR (KBr) νₘₐₓ 1611, 1578, 1477, 1441, 1388, 1328, 1308, and 1228 cm⁻¹; MS (ESI) m/z (rel intensity) 293 (100%, MH⁺); HRMS calcd for C₁₉H₂₁N₂O (MH⁺) 293.1654, found 293.1640; Anal. Calcd for C₁₉H₂₀N₂O: C, 78.05; H, 6.89; N, 9.58. Found: C, 77.70; H, 6.97; N, 9.62.

Single crystals of compound 9 suitable for X-ray analysis were grown by slow evaporation of its CH₂Cl₂-EtOAc solution.

### 6-tert-Butyl-11-methyl-6,7-dihydro-5-oxa-7a,12-diazadibenzo[a,e]azulene (11)

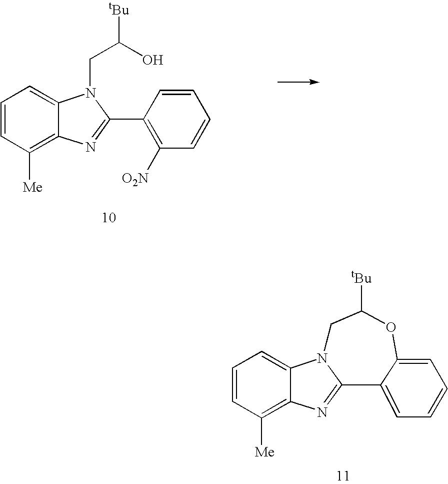

To a solution of alcohol 10 (100 mg, 0.28 mmol) in anhydrous DMF (2 mL) was added NaH (60% w/w, 12.5 mg, 0.31 mmol) to give a dark-brown solution. After 13 h at rt, the reaction mixture was quenched with water, and diluted with EtOAc. The organic phase was washed repeatedly with water, dried (MgSO₄), and evaporated in vacuo to give a yellow oil. Purification by flash chromatography (silica gel, CH₂Cl₂→CH₂Cl₂/EtOAc, 5/1) gave the title compound 11 (83 mg, 96%) as a clear oil that solidified on standing: R_{f}=0.55 (petroleum ether/EtOAc, 9/1); ¹H NMR (400 MHz, CDCl₃) δ 1.07 (s, 9H), 2.65 (s, 3H), 3.79 (dd, J=9.5, 1.0 Hz, 1H), 4.15 (dd, J=13.5, 9.5 Hz, 1H), 4.45 (dd, J=13.5, 1.0 Hz, 1H), 6.96-7.12 (m, 5H), 7.24 (dt, J=7.5, 1.5 Hz, 1H), and 8.67 (dd, J=8.0, 1.5 Hz, 1H); ¹³C{¹H} NMR (101 MHz, CDCl₃) δ 17.10, 26.58, 35.37, 47.97, 87.53, 107.0, 120.3, 120.8, 123.0, 123.4 (2×C), 130.4, 131.5, 131.9, 136.7, 142.7, 149.7, and 157.6; IR (CHCl₃) νₘₐₓ 2965, 1603, 1576, 1475, 1446, 1373, and 1273 cm⁻¹; MS (ESI) m/z (rel intensity) 307 (100%, MH⁺); HRMS calcd for C₂₀H₂₃N₂O (MH⁺) 307.1810, found 307.1817.

### Ethyl 6-tert-butyl-6,7-dihydro-5-oxa-7a,12-diazadibenzo[a,e]azulene-11-carboxylate (13)

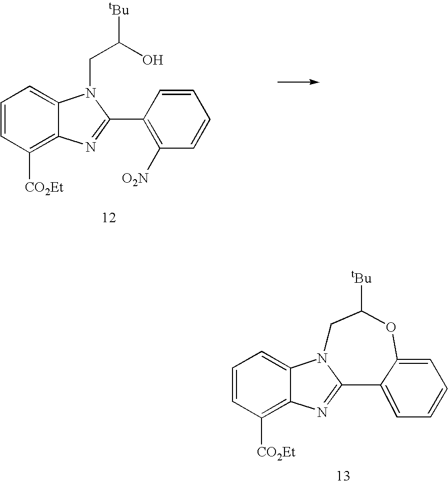

To a solution of alcohol 12^{xx} (100 mg, 0.24 mmol) in anhydrous DMF (2 mL) was added NaH (60% w/w, 10.8 mg, 0.27 mmol). After 20 h at rt, the reaction mixture was quenched with water, and diluted with EtOAc. The organic phase was washed repeatedly with water, dried (MgSO₄), and evaporated in vacuo to give a clear oil. Purification by flash chromatography (silica gel, CH₂Cl₂→CH₂Cl₂/EtOAc, 4/1) gave the title compound 13 (79 mg, 89%) as a clear oil that solidified on standing: R_{f}=0.55 (petroleum ether/EtOAc, 3/1); ¹H NMR (250 MHz, CDCl₃) δ 1.20 (s, 9H), 1.54 (t, J=7.0 Hz, 3H), 3.91 (˜d, J=9.5 Hz, 1H), 4.32 (dd, J=13.5, 9.5 Hz, 1H), 4.56 (q, J=7.0 Hz, 2H), 4.61 (d, J=13.5 Hz, 1H), 7.11 (d, J=8.0 Hz, 1H), 7.22 (dt, J=7.0, 1.0 Hz, 1H), 7.28-7.44 (m, 2H), 7.53 (d, J=8.0 Hz, 1H), 8.01 (d, J=7.5 Hz. 1H), and 8.89 (dd, J=8.0, 1.5 Hz, 1H); ¹³C{¹H} NMR (63 MHz, CDCl₃) δ 14.92, 26.56, 35.37, 48.29, 61.41, 87.18, 114.0, 119.6, 120.6, 121.7, 122.1, 123.5, 126.0, 132.2, 132.5, 138.3, 142.4, 152.1, 158.0, and 166.7; IR (CHCl₃) νₘₐₓ 2966, 1708, 1608, 1476, 1427, 1299, and 1256 cm⁻¹; MS (ESI) m/z (rel intensity) 365 (100%, MH⁺), 337 (35), and 319 (20); HRMS calcd for C₂₂H₂₄N₂NaO₃ (MNa⁺) 387.1685, found 387.1692.

### 6-tert-Butyl-11-[1,3]dioxolan-2-yl-6,7-dihydro-5-oxa-7a,12-diazadibenzo[a,e]azulene (3)

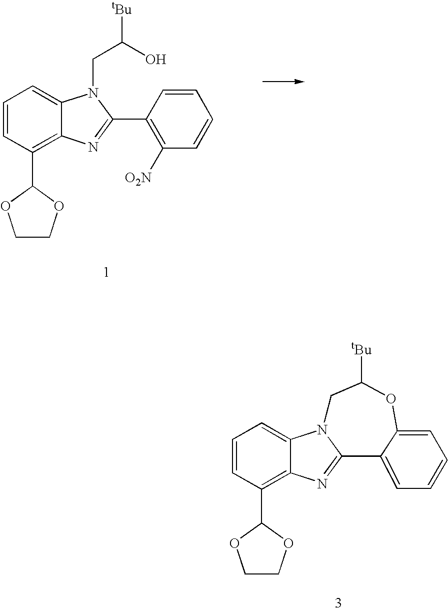

To a solution of alcohol 1^{xx} (200 mg, 0.49 mmol) in anhydrous DMF (2 mL) was added NaH (60% w/w, 21.6 mg, 0.54 mmol). After 19 h at rt, the reaction mixture was quenched with water, and diluted with EtOAc. The organic phase was washed repeatedly with water, dried (MgSO₄), and evaporated in vacuo to give an off-white solid. Purification by flash chromatography (silica gel, CH₂Cl₂→CH₂Cl₂/EtOAc, 10/1) gave the title compound 3 (161 mg, 92%) as a white solid: R_{f}=0.35 (petroleum ether/EtOAc, 3/1); mp 200.0-201.0° C. (EtOAc/petroleum ether); ¹H NMR (250 MHz, CDCl₃) δ 1.13 (s, 9H), 3.84 (dd, J=9.0, 1.0 Hz, 1H), 4.05-4.32 (m, 5H), 4.52 (dd, J=13.5, 1.0 Hz, 1H), 6.65 (s, 1H), 7.04 (dd, J=8.0, 1.0 Hz, 1H), 7.12 (dt, J=8.0, 1.0 Hz, 1H), 7.19-7.35 (m, 3H), 7.46 (dd, J=6.5, 1.5 Hz, 1H), and 8.75 (dd, J=8.0, 1.5 Hz, 1H); ¹³C{¹H} NMR (63 MHz, CDCl₃) δ 26.57, 35.35, 48.01, 66.07 and 66.11 (rotamers?), 87.44, 101.3, 110.4, 120.0, 120.2, 120.7, 122.8, 123.3, 129.1, 131.7, 132.4, 137.4, 141.7, 150.8, and 157.7; IR (CHCl₃) νₘₐₓ 2965, 1610, 1576, 1475, 1437, and 1076 cm⁻¹; MS (ESI) m/z (rel intensity) 365 (100%, MH⁺) and 321 (15); HRMS calcd for C₂₀H₂₅N₂O₃ (MH⁺) 365.1865, found 365.1870; Anal. Calcd for C₂₂H₂₄N₂O₃: C, 72.50; H, 6.64; N, 7.69. Found: C, 72.34; H, 6.68; N, 7.61. The cyclic ether 3 was optically resolved by CSP HPLC (FIG. S1).

### 6,6,11-Trimethyl-6,7-dihydro-5-oxa-7a,12-diazadibenzo[a,e]azulene (15)

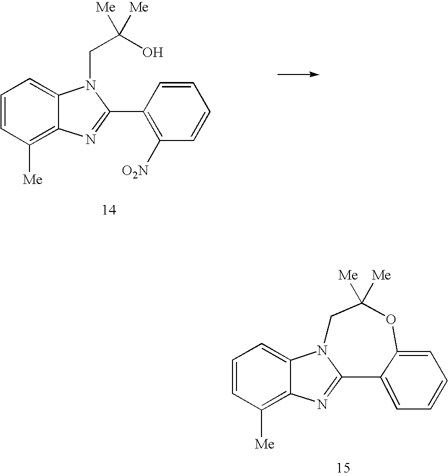

To a solution of alcohol 14 (100 mg, 0.31 mmol) in anhydrous DMF (1 mL) was added NaH (95% w/w, 8.6 mg, 0.34 mmol) to give a brown solution. After 2 h at rt, the reaction mixture was quenched with water, and diluted with EtOAc. The organic phase was washed repeatedly with water, dried (MgSO₄), and evaporated in vacuo to give a pale brown oil. Purification by flash chromatography (silica gel, CH₂Cl₂→CH₂Cl₂/EtOAc, 8/1) gave the title compound 15 (58 mg, 68%) as a clear oil: R_{f}=0.40 (petroleum ether/EtOAc, 3/1); ¹H NMR (400 MHz, CDCl₃) δ 1.48 (s, 6H), 2.79 (s, 3H), 4.05 (s, 2H), 7.09-7.32 (m, 5H), 7.45 (˜t, J=8.0 Hz, 1H), and 8.20 (d, J=7.5 Hz, 1H); ¹³C{¹H} NMR (63 MHz, CDCl₃) δ 17.31, 25.64, 52.12, 84.76, 106.5, 123.1 (2×C?), 124.1, 124.2, 124.6, 130.6, 130.8, 131.9, 135.9, 143.0, 151.8, and 153.7; IR (CHCl₃) νₘₐₓ 1610, 1469, 1455, and 1386 cm⁻¹; MS (ESI) m/z (rel intensity) 301 (90%, MNa⁺) and 279 (100); HRMS calcd for C₁₈H₁₉N₂O (MH⁺) 279.1497, found 279.1494.

### 6-tert-Butyl-4-chloro-11-methyl-6,7-dihydro-5-oxa-7a,12-diazadibenzo[a,e]azulene (17)

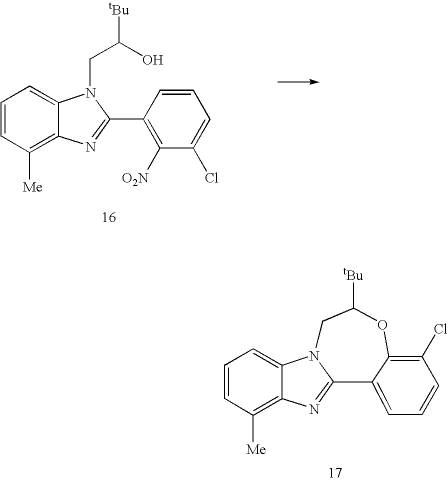

To a solution of alcohol 16 (100 mg, 0.26 mmol) in anhydrous DMF (2 mL) was added NaH (60% w/w, 11.5 mg, 0.29 mmol). After 23 h at rt, the reaction mixture was quenched with water, and diluted with EtOAc. The organic phase was washed repeatedly with water, dried (MgSO₄), and evaporated in vacuo to give a white foam. Purification by flash chromatography (silica gel, CH₂Cl₂→CH₂Cl₂/EtOAc, 10/1) gave the title compound 17 (70 mg, 80%) as a white solid: R_{f}=0.55 (petroleum ether/EtOAc, 9/1); mp 191.0-192.0° C. (EtOAc/petroleum ether); ¹H NMR (400 MHz, CDCl₃) δ 1.24 (s, 9H), 2.74 (s, 3H), 3.98 (d, J=8.5 Hz, 1H), 4.26 (dd, J=14.0, 8.5 Hz, 1H), 4.57 (d, J=14.0 Hz, 1H), 7.05-7.25 (m, 4H), 7.45 (dd, J=7.5, 1.5 Hz, 1H), and 8.68 (dd, J=8.0, 1.5 Hz, 1H); ¹³C{¹H} NMR (101 MHz, CDCl₃) δ 16.90, 26.62, 35.25, 47.15, 88.78, 106.8, 121.2, 123.1 (2×C), 123.3, 126.0, 130.3, 130.4, 131.9, 136.3, 142.5, 148.8, and 152.3; IR (CHCl₃) νₘₐₓ 2965, 1468, 1422, and 1369 cm⁻¹; MS (ESI) m/z (rel intensity) 341 (100%, MH⁺); HRMS calcd for C₂₀H₂₂ClN₂O (MH⁺) 341.1420, found 341.1407; Anal. Calcd for C₂₀H₂₁ClN₂O: C, 70.48; H, 6.21; Cl, 10.40; N, 8.22. Found: C, 70.43; H, 6.18; Cl, 10.53; N, 8.26.

### 6-tert-Butyl-4,11-dimethyl-6,7-dihydro-5-oxa-7a,12-diazadibenzo[a,e]azulene (19)

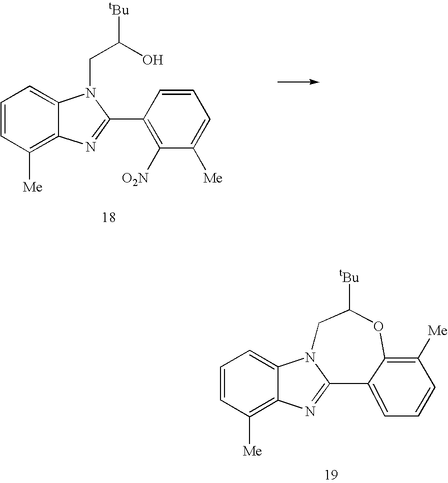

To a solution of alcohol 18 (200 mg, 0.54 mmol) in anhydrous DMF (2 mL) was added NaH (95% w/w, 15.1 mg, 0.6 mmol) to give a deep-violet solution. After 22 h at rt, the reaction mixture was quenched with water, and diluted with EtOAc. The organic phase was washed repeatedly with water, dried (MgSO₄), and evaporated in vacuo to give a brown oil. Purification by flash chromatography (silica gel, CH₂Cl₂→CH₂Cl₂/EtOAc, 10/1) gave the title compound 19 (13 mg, 7%) as a white solid: R_{f}=0.55 (petroleum ether/EtOAc, 9/1); ¹H NMR (400 MHz, CDCl₃) δ 1.18 (s, 9H), 2.37 (s, 3H), 2.73 (s, 3H), 3.92 (d, J=7.5 Hz, 1H), 4.22 (dd, J=14.0, 7.5 Hz, 1H), 4.55 (d, J=14.0 Hz, 1H), 7.03-7.22 (m, 5H), and 8.55 (d, J=8.0 Hz, 1H); ¹³C{¹H} NMR (101 MHz, CDCl₃) δ 16.73, 17.36, 26.44, 34.92, 46.64, 87.49, 106.3, 118.4, 122.2, 122.4, 122.9, 129.3, 129.5, 129.9, 132.6, 135.8, 142.2, 150.1, and 154.5; IR (CHCl₃) νₘₐₓ 2965, 1601, 1475, and 1417 cm⁻¹; MS (ESI) m/z (rel intensity) 321 (100%, MH⁺); HRMS calcd for C₂₁H₂₄N₂NaO (MNa⁺) 343.1786, found 343.1772.

### 6-tert-Butyl-11-methyl-6,7-dihydro-5-oxa-7a,12-diazadibenzo[a,e]azulene (11)

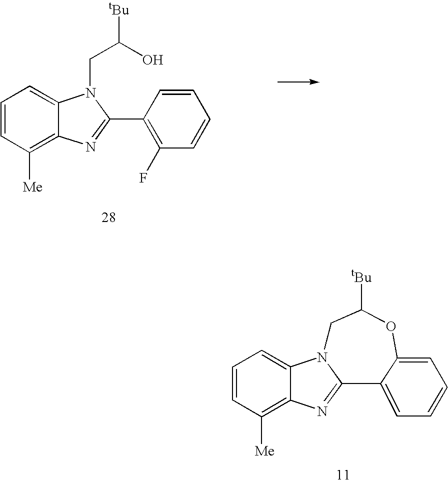

To a solution of alcohol 28 (100 mg, 0.31 mmol) in anhydrous DMF (2 mL) was added NaH (60% w/w, 13.5 mg, 0.34 mmol). After 5 h at rt, the reaction mixture was quenched with water, and diluted with EtOAc. The organic phase was washed repeatedly with water, dried (MgSO₄), and evaporated in vacuo. Purification by flash chromatography (silica gel, CH₂Cl₂→CH₂Cl₂/EtOAc, 5/1) gave the title compound 11 (87 mg, 93%) identical (TLC, ¹H NMR) with the product of the nitro-group displacement from alcohol 10 (vide supra).

### Attempted Replacement of the Chloro Group in Alcohol (29)

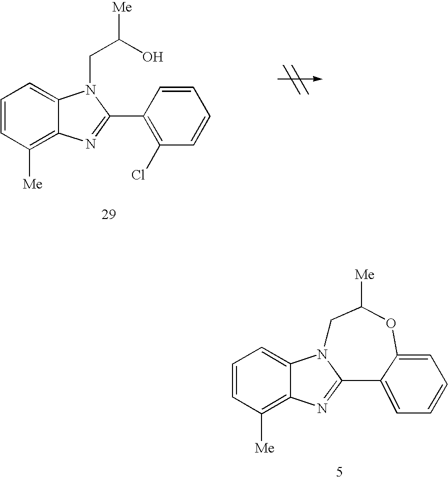

To a solution of alcohol 29 (136 mg, 0.45 mmol) in anhydrous DMF (2 mL) was added NaH (95% w/w, 12.6 mg, 0.50 mmol). After 24 h at rt, the reaction mixture was quenched with water, neutralized with 5% HCl, and diluted with EtOAc. The organic phase was washed repeatedly with water, dried (MgSO₄), and evaporated in vacuo. ¹H NMR analysis of the crude product indicated the presence of the starting material 29, only. Purification by flash chromatography (silica gel, CH₂Cl₂→EtOAc) gave the recovered starting material 29 (107 mg, 79%).

### (E/Z)-2-(4-Methyl-1-propenyl-1H-benzimidazol-2-yl)phenol (30)

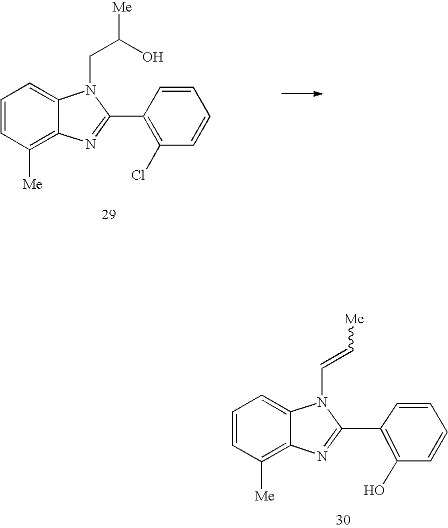 Method 1:

To a solution of alcohol 29 (107 mg, 0.36 mmol) in anhydrous DMF (1 mL) was added NaH (95% w/w, 9.9 mg, 0.4 mmol). After 24 h at 90° C., the reaction mixture was quenched with water, neutralized with 5% HCl, and diluted with EtOAc. The organic phase was washed repeatedly with water, dried (MgSO₄), and evaporated in vacuo to give a pale brown oil. Purification by flash chromatography (silica gel, CH₂Cl₂→EtOAc) gave the title compound 30 (37 mg, 39%, E/Z=˜2:1), cyclic ether 5 (˜3 mg, ˜3%), and recovered starting material 29 (45 mg, 42%). Phenol 30: a white solid, MS (ESI) m/z (rel intensity) 287 (60%, MNa⁺) and 265 (100); HRMS calcd for C₁₇H₁₆N₂NaO (MNa⁺) 287.1160, found 287.1158.

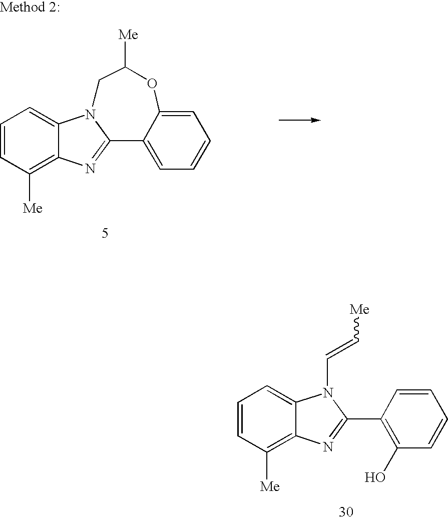

To a solution of the cyclic ether 5 (54 mg, 0.20 mmol) in anhydrous DMF (1 mL) was added NaH (95% w/w, 5.7 mg, 0.2 mmol). After 18 h at 90° C., the reaction mixture was quenched with water, neutralized with 5% HCl, and diluted with EtOAc. The organic phase was washed repeatedly with water, dried (MgSO₄), and evaporated in vacuo to give an off-white solid. Purification by flash chromatography (silica gel, CH₂Cl₂→EtOAc) gave the title compound 30 (46 mg, 85%, E/Z=˜2:1) as a white solid.

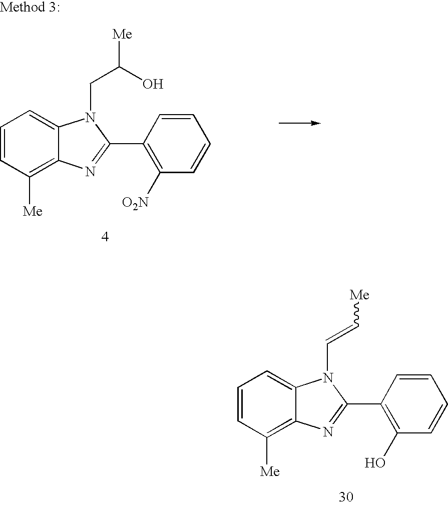

To a solution of alcohol 4 (100 mg, 0.32 mmol) in anhydrous DMF (1 mL) was added NaH (95% w/w, 17.9 mg, 0.7 mmol). After 1 h at rt, followed by 23 h at 90° C., the reaction mixture was quenched with water, neutralized with 5% HCl, and diluted with EtOAc. The organic phase was washed repeatedly with water, dried (MgSO₄), and evaporated in vacuo to give a brown oil. Purification by flash chromatography (silica gel, CH₂Cl₂) gave the title compound 30 (60 mg, 71%, E/Z=˜2.2:1) as a white solid. The two isomeric products were separated by repeated purification by flash chromatography (silica gel, petroleum ether/CH₂Cl₂, 1/1). Phenol (Z)-30: a white solid: R_{f}=0.60 (petroleum ether/CH₂Cl₂, 1/1); ¹H NMR (400 MHz, CDCl₃) δ 1.58 (dd, J=7.0, 2.0 Hz, 3H), 2.67 (s, 3H), 6.15 (dq, J=8.0, 7.0 Hz, 1H), 6.83 (dq, J=8.0, 2.0 Hz, 1H), 6.87 (˜dt, J=7.5, 1.0 Hz, 1H), 7.05 (d, J=8.0 Hz, 1H), 7.11 (dd, J=8.5, 1.0 Hz, 1H), 7.12 (˜dd, J=7.5, 1.0 Hz, 1H), 7.21 (t, J=8.0 Hz, 1H), 7.32 (ddd, J=8.5, 7.5, 1.5 Hz, 1H), 8.08 (dd, J=8.5, 1.5 Hz, 1H), and 13.7 (br s, 1H); ¹³C{¹H} NMR (101 MHz, CDCl₃) δ 12.92, 16.54, 108.2, 113.4, 117.9, 118.3, 123.6, 124.6, 126.8, 128.9, 129.3, 131.5, 134.0, 139.4, 149.9, and 159.5 (one C atom obscured); IR (CHCl₃) νₘₐₓ 1623, 1583, 1484, 1375, 1363, 1271, and 1255 cm⁻¹; MS (ESI) m/z (rel intensity) 265 (100%, MH⁺) and 225 (90); HRMS calcd for C₁₇H₁₇N₂O (MH⁺) 265.1341, found 265.1330. Phenol (E)-30: a white solid: R_{f}=0.55 (petroleum ether/CH₂Cl₂, 1/1); ¹H NMR (400 MHz, CDCl₃) δ 2.04 (dd, J=7.0, 1.5 Hz, 3H), 2.66 (s, 3H), 6.19 (dq, J=14.0, 7.0 Hz, 1H), 6.80 (˜dd, J=14.0, 1.5 Hz, 1H), 6.91 (t, J=7.5 Hz, 1H), 7.09-7.16 (m, 2H), 7.20 (t, J=7.5 Hz, 1H), 7.28 (d, J=8.0 Hz, 1H), 7.34 (dt, J=8.0, 1.5 Hz, 1H), 7.93 (d, J=8.0 Hz, 1H), and 13.4 (br s, 1H); ¹³C{¹H} NMR (101 MHz, CDCl₃) δ 15.36, 16.50, 108.1, 113.3, 117.9, 118.3, 123.5, 123.6, 125.1, 127.3, 127.5, 128.8, 131.4, 134.5, 139.7, 149.7, and 159.3; IR (CHCl₃) νₘₐₓ 1624, 1584, 1485, 1385, 1373, 1270, and 1256 cm⁻¹; MS (ESI) m/z (rel intensity) 265 (100%, MH⁺) and 225 (60); HRMS calcd for C₁₇H₁₇N₂O (MNH⁺) 265.1341, found 265.1323.

### Intramolecular S_{N}Ar Reactions with Diols

### (6,7-Dihydro-5-oxa-7a,12-diazadibenzo[a,e]azulen-6-yl)methanol (22)

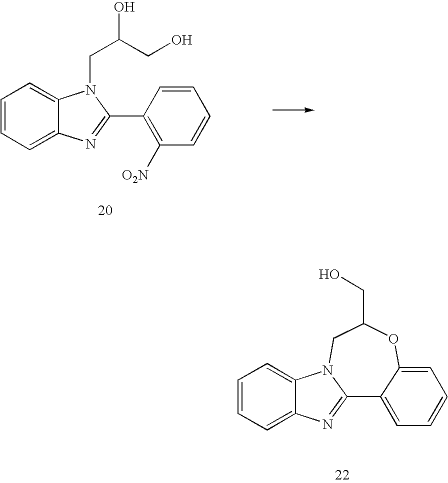

To a solution of diol 20 (1.00 g, 3.19 mmol) in anhydrous DMF (10 mL) was added NaH (60% w/w, 281 mg, 7.0 mmol) to give a dark-brown solution. After 100 min at rt, the reaction mixture was quenched with water, and diluted with EtOAc. The organic phase was washed repeatedly with water, dried (MgSO₄), and evaporated in vacuo to give an off-white solid. Purification by flash chromatography (silica gel, CH₂Cl₂→CH₂Cl₂/EtOAc, 1/1) gave the title compound 22 (818 mg, 96%) as a white solid: R_{f}=0.65 (EtOAc); mp 142.0-143.5° C. (EtOAc/petroleum ether); [α]_{D}=−0.7 (c 0.88 in CHCl₃); ¹H NMR (400 MHz, CDCl₃) δ 3.45 (br s, 1H), 3.98 (dd, J=11.5, 5.0 Hz, 1H), 4.04 (dd, J=11.5, 5.0 Hz, 1H), 4.35 (dd, J=13.0, 8.5 Hz, 1H), 4.39-4.44 (m, 1H), 4.55 (dd, J=13.0, 0.5 Hz, 1H), 7.05 (dd, J=8.0, 1.0 Hz, 1H), 7.20 (dt, J=8.0, 1.0 Hz, 1H), 7.28-7.37 (m, 4H), 7.84-7.86 (m, 1H), and 8.60 (dd, J=8.0, 1.5 Hz, 1H); ¹³C{¹H} NMR (63 MHz, CDCl₃) δ 47.37, 63.15, 80.17, 109.2, 119.3, 119.5, 121.0, 122.9, 123.0, 123.5, 131.2, 131.5, 136.1, 142.5, 150.1, and 155.7; IR (CHCl₃) νₘₐₓ 2963, 1611, 1576, 1476, 1445, 1387, and 1324 cm⁻¹; MS (ESI) m/z (rel intensity) 267 (100%, MH⁺); HRMS calcd for C₁₆H₁₅N₂O₂ (MH⁺) 267.1133, found 267.1122; Anal. Calcd for C₁₆H₁₄N₂O₂: C, 72.16; H, 5.30; N, 10.52. Found: C, 72.00; H, 5.19; N, 10.55. Alcohol 22 was optically resolved by CSP HPLC (FIG. S2).

### (6,7-Dihydro-5-oxa-7a,12-diazadibenzo[a,e]azulen-6-yl)methanol (22) and 6,7-dihydro-5-oxa-7a,12-diazadibenzo[a,e]azulen-6-ylmethyl acetate (58)

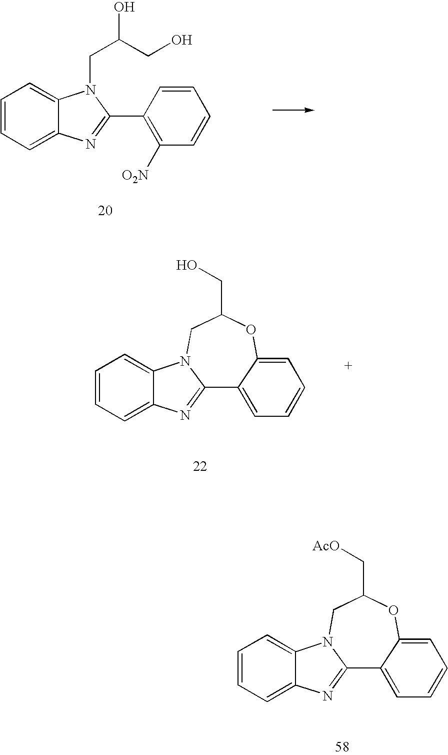

To a solution of diol 20 (1.00 g, 3.19 mmol) in anhydrous DMF (10 mL) was added NaH (60% w/w, 281 mg, 7.0 mmol) to give a dark-brown solution. After 2 h at rt, the reaction mixture was diluted with EtOAc, and washed repeatedly with water. The organic layer was dried (MgSO₄), and evaporated in vacuo to give an off-white oil. Purification by flash chromatography (silica gel, CH₂Cl₂→CH₂Cl₂/EtOAc, 1/1) gave the title compound 58 (301 mg, 31%) as a white semi-solid, and alcohol 22 (588 mg, 69%) as a white solid. Ester 58: R_{f}=0.75 (CH₂Cl₂/EtOAc, 2/1); 1H NMR (250 MHz, CDCl₃) δ 1.97 (s, 3H), 4.12 (dd, J=14.0, 8.5 Hz, 1H), 4.20 (dd, J=11.0, 5.0 Hz, 1H), 4.29 (dd, J=14.0, 2.0 Hz, 1H), 4.35 (dd, J=11.5, 5.0 Hz, 1H), 4.41 (dddd, J=8.5, 5.0, 5.0, 2.0 Hz, 1H), 6.95 (dd, J=8.0, 1.0 Hz, 1H), 7.05 (dt, J=8.0, 1.0 Hz, 1H), 7.10-7.26 (m, 4H), 7.65-7.69 (m, 1H), and 8.44 (dd, J=8.0, 1.5 Hz, 1H); ¹³C{¹H} NMR (63 MHz, CDCl₃) δ 20.57, 47.23, 63.76, 77.42, 108.9, 119.5 (2×C), 121.0, 122.7, 122.8, 123.6, 131.0, 131.4, 135.9, 142.4, 149.8, 155.3, and 170.3; IR (CHCl₃) νₘₐₓ 1744, 1475, and 1445 cm⁻¹; MS (ESI) m/z (rel intensity) 309 (100%, MH⁺); HRMS calcd for C₁₈H₁₆N₂NaO₃ (MNa⁺) 331.1059, found 331.1064. Acetate 58 was optically resolved by CSP HPLC (FIG. S5).

### (6,7-Dihydro-5-oxa-7a,12-diazadibenzo[a,e]azulen-6-yl)methanol (22)

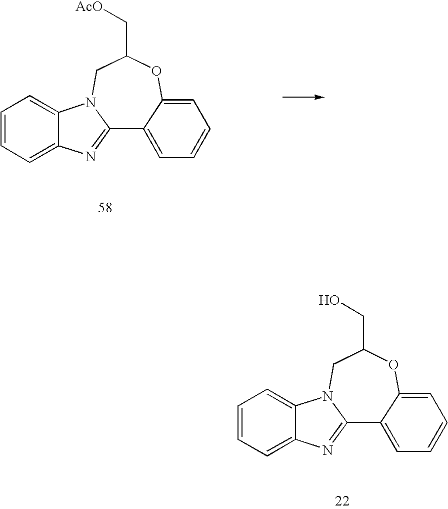

To a solution of ester 58 (128 mg, 0.42 mmol) in THF (4 mL) was added 1M LiOH (1.3 mL, 1.3 mmol). The reaction mixture was stirred at rt for 17 h, and partitioned between CH₂Cl₂ and water. The phases were separated, and the extraction was completed with additional portions of CH₂Cl₂. The combined organic extracts were dried (MgSO₄), and evaporated in vacuo to give a white solid. Purification by flash chromatography (silica gel, CH₂Cl₂/EtOAc, 1/1) gave the title compound 22 (104 mg, 95%), identical (TLC, ¹H NMR) with the product obtained from the cyclization of diol 20.

### 6,7-Dihydro-5-oxa-7a,12-diazadibenzo[a,e]azulen-6-ylmethyl toluene-4-sulfonate (59)

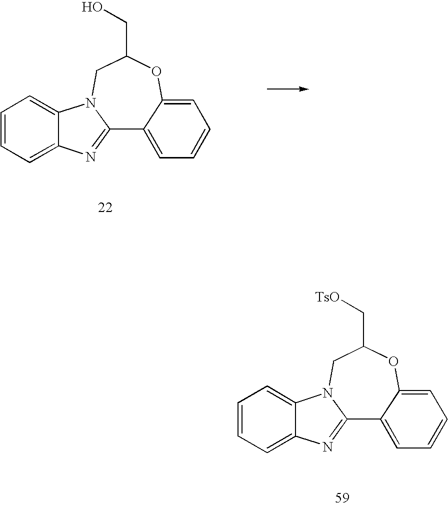

To an ice-cooled solution of alcohol 22 (266 mg, 1.00 mmol) in anhydrous pyridine (1 mL) was added TsCl (238 mg, 1.25 mmol). After 14 h at 0° C.→rt, the reaction mixture was partitioned between CH₂Cl₂ and water, and neutralized with 1M HCl. The phases were separated and the extraction was completed with additional portions of CH₂Cl₂. The combined organic extracts were dried (MgSO₄) and evaporated in vacuo to give a white solid. Purification by flash chromatography (silica gel, CH₂Cl₂→CH₂Cl₂/EtOAc, 2/1) gave the title compound 59 (397 mg, 95%) as a white solid: R_{f}=0.25 (petroleum ether/EtOAc, 2/1); mp 165.5-166.5° C. (EtOAc/petroleum ether); ¹H NMR (400 MHz, CDCl₃) δ 2.46 (s, 3H), 4.27 (dd, J=10.5, 6.0 Hz, 1H), 4.31 (dd, J=14.0, 8.0 Hz, 1H), 4.41 (dd, J=10.5, 5.0 Hz, 1H), 4.47 (dd, J=14.0, 2.0 Hz, 1H), 4.61 (dddd, J=8.0, 6.0, 5.0, 2.0 Hz, 1H), 6.99 (d, J=8.0 Hz, 1H), 7.21 (dt, J=8.0, 1.0 Hz, 1H), 7.28-7.38 (m, 6H), 7.80-7.82 (m, 1H), 7.81 (d, J=8.0 Hz, 2H), and 8.52 (dd, J=8.0, 1.5 Hz, 1H); ¹³C{¹H} NMR (101 MHz, CDCl₃) δ 21.62, 46.43, 68.33, 77.20, 109.0, 119.7 (2×C), 121.2, 122.8, 123.0, 123.9, 127.9, 130.0, 131.1, 131.5, 132.2, 135.9, 142.7, 145.4, 149.9, and 154.6; IR (CHCl₃) νₘₐₓ 1611, 1599, 1577, 1475, 1446, 1371, and 1191 cm⁻¹; MS (ESI) m/z (rel intensity) 421 (100%, MH⁺); HRMS calcd for C₂₃H₂₀N₂NaO₄S (MNa⁺) 443.1041, found 443.1046; Anal. Calcd for C₂₃H₂₀N₂O₄S: C, 65.70; H, 4.79; N, 6.66; S, 7.63. Found: C, 65.71; H, 4.79; N, 6.65; S, 7.55.

### 6-Methyl-6,7-dihydro-5-oxa-7a,12-diazadibenzo[a,e]azulene (23)

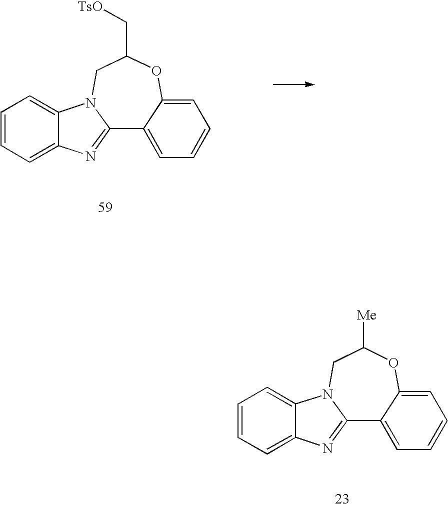

To an ice-cooled solution of tosylate 59 (100 mg, 0.24 mmol) in THF (2 mL) was added LiAlH₄ (76 mg, 2.0 mmol). After 1.5 h at 0° C.→rt, the reaction mixture was re-cooled in an ice bath, and quenched by a slow addition of water (80 μL), 15% NaOH (240 μL), and water (240 μL). The resulting thick suspension was diluted with CH₂Cl₂, and filtered through a thin pad of Celite®. The filtrate was evaporated in vacuo to give a clear oil. Purification by flash chromatography (silica gel, petroleum ether/EtOAc, 2/1) gave the title compound 23 (47 mg, 79%) as a white solid: R_{f}=0.25 (petroleum ether/EtOAc, 2/1); ¹H NMR (250 MHz, CDCl₃) 1.46 (d, J=6.5 Hz, 3H), 4.09 (dd, J=14.0, 8.5 Hz, 1H), 4.27 (dd, J=14.0, 2.0 Hz, 1H), 4.43 (ddq, J=8.5, 6.5, 2.0 Hz, 1H), 6.96 (dd, J=8.0, 1.0 Hz, 1H), 7.06 (dt, J=8.0, 1.5 Hz, 1H), 7.12-7.26 (m, 4H), 7.69-7.73 (m, 1H), and 8.53 (dd, J=8.0, 1.5 Hz, 1H); ¹³C{¹H} NMR (63 MHz, CDCl₃) δ 19.39, 51.52, 75.84, 109.0, 119.5, 119.6, 121.2, 122.7 (2×C?), 123.1, 131.2, 131.3, 136.2, 142.7, 150.3, and 155.9; IR (CHCl₃) νₘₐₓ 1609, 1576, 1476, 1449, 1385, 1325, and 1227 cm⁻¹; MS (ESI) m/z (rel intensity) 251 (100%, MH⁺); HRMS calcd for C₁₆H₁₅N₂O (MH⁺) 251.1184, found 251.1192.

### (4-Chloro-11-methyl-6,7-dihydro-5-oxa-7a,12-diazadibenzo[a,e]azulen-6-yl)methanol (25) and cyclic ether (26)

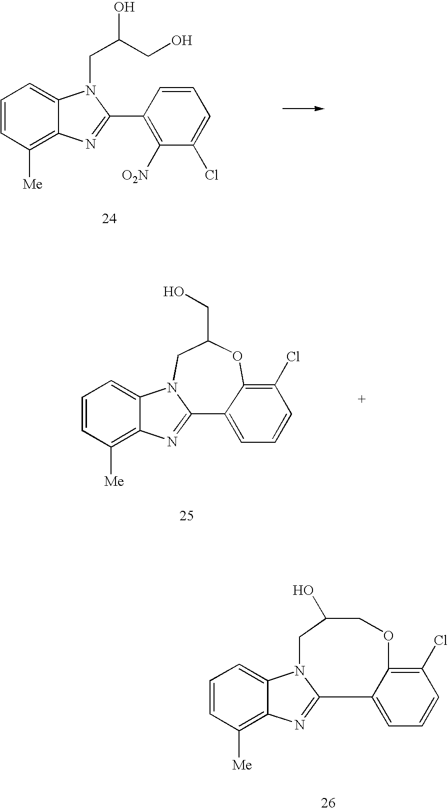

To a solution of diol 24 (642 mg, 1.78 mmol) in anhydrous DMF (4 mL) was added NaH (95% w/w, 95 mg, 3.9 mmol) to give a yellow solution. After 1 h at rt, the reaction mixture was quenched with water, and diluted with CH₂Cl₂. The organic phase was washed repeatedly with water, dried (MgSO₄), and evaporated in vacuo to give a white solid. Purification by flash chromatography (silica gel, CH₂Cl₂→CH₂Cl₂/EtOAc, 1/1) gave the title compounds 25 (389 mg, 70%) and 26 (45 mg, 8%) as white solids. Benzimidazole 25: R_{f}=0.70 (CH₂Cl₂/EtOAc, 3/1); mp 186.5-188.0° C. (EtOAc/petroleum ether); ¹H NMR (400 MHz, d₆-DMSO) δ 2.61 (s, 3H), 3.78^{xxi} (ddd, J=11.5, 6.0, 5.5 Hz, 1H), 3.88^{xxi} (ddd, J=11.5, 5.5, 5.0 Hz, 1H), 4.44 (dd, J=14.5, 8.0 Hz, 1H), 4.56 (dddd, J=8.0, 6.0, 5.0, 2.0 Hz, 1H), 4.69 (dd, J=14.5, 2.0 Hz, 1H), 5.32 [t, J=5.5 Hz, 1H (D₂O exchangeable)], 7.19 (d, J=8.0 Hz, 1H), 7.20 (t, J=8.0 Hz, 1H), 7.23 (t, J=8.0 Hz, 1H), 7.41 (d, J=8.0 Hz, 1H), 7.61 (dd, J=8.0, 1.5 Hz, 1H), and 8.44 (dd, J=8.0, 1.5 Hz, 1H); ¹³C{¹H} NMR (101 MHz, d₆-DMSO) δ 16.33, 46.68, 61.52, 82.11, 107.7, 122.3, 122.6, 122.7, 123.7, 125.5, 128.7, 129.5, 131.3, 135.9, 141.5, 148.0, and 151.0; IR (KBr) νₘₐₓ 1472, 1459, and 1246 cm⁻¹; MS (ESI) m/z (rel intensity) 337 (30%, MNa⁺) and 315 (100); HRMS calcd for C₁₇H₁₆ClN₂O₂ (MH⁺) 315.0900, found 315.0903; Anal. Calcd for C₁₇H₁₅ClN₂O₂: C, 64.87; H, 4.80; Cl, 11.26; N, 8.90. Found: C, 64.90; H, 4.83; Cl, 11.22; N, 8.84. Benzimidazole 26: R_{f}=0.60 (CH₂Cl₂/EtOAc, 3/1); mp>260° C. (EtOAc/petroleum ether); ¹H NMR (400 MHz, d₆-DMSO) δ 2.58 (s, 3H), 3.98-4.04 (m, 2H), 4.23 (d, J=3.5 Hz, 1H), 4.29 and 4.30 (ABq, J=14.5 Hz, 2H), 5.49 [(d, J=4.0 Hz, 1H (D₂O exchangeable)], 7.06 (d, J=7.4 Hz, 1H), 7.20 (t, J=7.5 Hz, 1H), 7.30 (t, J=8.0 Hz, 1H), 7.46 (d, J=8.0 Hz, 1H), 7.71 (dd, J=7.5, 1.5 Hz, 1H), and 7.72 (dd, J=8.0, 1.5 Hz, 1H); ¹³C{¹H} NMR (101 MHz, d₆-DMSO) δ 16.35, 47.36, 65.56, 75.70, 108.3, 122.0, 122.5, 124.2, 124.7, 126.3, 128.6, 130.9, 132.1, 136.2, 141.8, 149.5, and 153.5; IR (KBr) νₘₐₓ 1597, 1471, 1454, 1432, 1397, 1265, 1068, and 994 cm⁻¹; MS (ESI) m/z (rel intensity) 337 (45%, MNa⁼) and 315 (100); HRMS calcd for C₁₇H₁₆ClN₂O₂(MH⁺) 315.0900, found 315.0890; Anal. Calcd for C₁₇H₁₅ClN₂O₂: C, 64.87; H, 4.80; Cl, 11.26; N, 8.90. Found: C, 64.44; H, 4.82; Cl, 11.32; N, 8.80. Alcohols 25 and 26 were optically resolved by CSP HPLC (FIGS. S3 and S4, respectively).

### 4-Chloro-11-methyl-6,7-dihydro-5-oxa-7a,12-diazadibenzo[a,e]azulen-6-ylmethyl toluene-4-sulfonate (60)

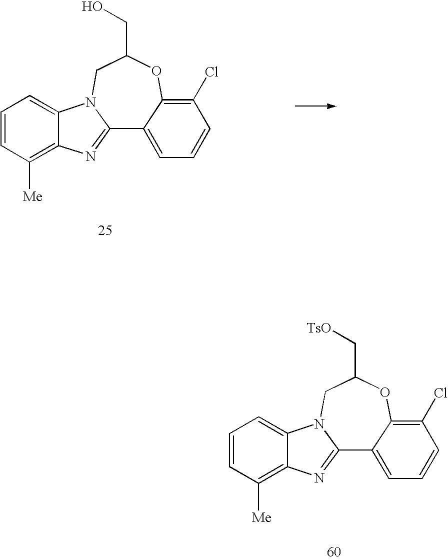

To an ice-cooled suspension of alcohol 25 (314 mg, 1.00 mmol) in anhydrous pyridine (2 mL) was added TsCl (238 mg, 1.25 mmol). After 17 h at 0° C.→rt, the reaction mixture was partitioned between CH₂Cl₂ and water, and neutralized with 1M HCl. The phases were separated and the extraction was completed with additional portions of CH₂Cl₂. The combined organic extracts were dried (MgSO₄) and evaporated in vacuo to give a white solid. Purification by flash chromatography (silica gel, CH₂Cl₂→CH₂Cl₂/EtOAc, 5/1) gave the title compound 60 (441 mg, 94%) as a white solid: R_{f}=0.50 (petroleum ether/EtOAc, 2/1); mp 168.0-169.0° C. (EtOAc/petroleum ether); ¹H NMR (250 MHz, CDCl₃) δ 2.32 (s, 3H), 2.58 (s, 3H), 4.12-4.36 (m, 4H), 4.52-4.60 (m, 1H), 6.97-7.14 (m, 4H), 7.22 (d, J=8.0 Hz, 1H), 7.31 (dd, J=8.0, 1.5 Hz, 1H), 7.69 (d, J=8.0 Hz, 1H), and 8.25 (dd, J=8.0, 1.5 Hz, 1H); ¹³C{¹H} NMR (63 MHz, CDCl₃) δ 17.05, 22.09, 46.41, 68.60, 79.05, 107.1, 123.6, 123.7, 123.8, 125.0, 127.1, 128.5, 130.1, 130.5, 130.7, 132.1, 132.6, 136.1, 142.6, 145.9, 148.8, and 150.6; IR (CHCl₃) νₘₐₓ 1469, 1450, 1432, 1373, 1228, and 1190 cm⁻¹; MS (ESI) m/z (rel intensity) 491 (60%, MNa⁺) and 469 (100); HRMS calcd for C₂₄H₂₁ClN₂NaO₄S (MNa⁺) 391.0808, found 391.0803; Anal. Calcd for C₂₄H₂₁ClN₂O₄S: C, 61.47; H, 4.51; Cl, 7.56; N, 5.97; S, 6.84. Found: C, 61.61; H, 4.49; Cl, 7.69; N, 5.93; S, 6.78.

22727/04187

### 8-(1-Chlorovinyl)-1,6,9-trimethyl-5,6-dihydro-7-oxa-4b,10-diazabenzo[a]azulene (27)

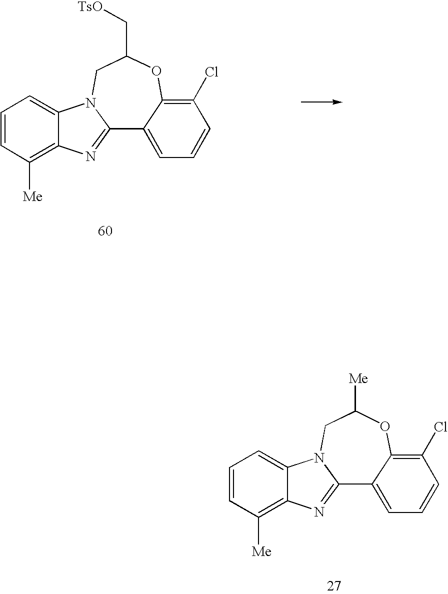

To an ice-cooled solution of tosylate 60 (100 mg, 0.21 mmol) in THF (2 mL) was added LiAlH₄ (40 mg, 1.1 mmol). After 3 h at 0° C.→rt, the reaction mixture was re-cooled to 0° C., and quenched by a slow addition of water (40 μL), 15% NaOH (120 μL), and water (40 μL). The resulting thick suspension was diluted with CH₂Cl₂, and filtered through a thin pad of Celite®. The filtrate was evaporated in vacuo to give a clear oil. Purification by flash chromatography (silica gel, petroleum ether/CH₂Cl₂, 1/1) gave the title compound 27 (59 mg, 93%) as a white solid: 1H NMR (400 MHz, CDCl₃) 1.67 (dt, J=6.5, 1.5 Hz, 3H), 2.75 (s, 3H), 4.25 (ddt, J=14.0, 8.5, 1.5 Hz, 1H), 4.38 (˜d, J=14.0 Hz, 1H), 4.66 (m, 1H), 7.11-7.28 (m, 4H), 7.48 (˜dd, J=8.0, 1.5 Hz, 1H), and 8.55 (dd, J=8.0, 1.5 Hz, 1H); ¹³C{¹H} NMR (63 MHz, CDCl₃) δ 16.61, 19.36, 51.12, 77.61, 106.5, 122.8, 123.0, 123.1, 123.7, 126.5, 129.7, 130.1, 131.4, 135.8, 142.0, 148.6, and 151.4; IR (CHCl₃) νₘₐₓ 1470, 1452, 1431, 1422, and 1385 cm⁻¹; MS (ESI) m/z (rel intensity) 321 (55%, MNa⁺) and 299 (100); HRMS calcd for C₁₇H₁₆ClN₂O (MH⁺) 299.0951, found 299.0930.

### (4-Chloro-6,7-dihydro-5-oxa-7a,12-diazadibenzo[a,e]azulen-6-yl)methanol (61)

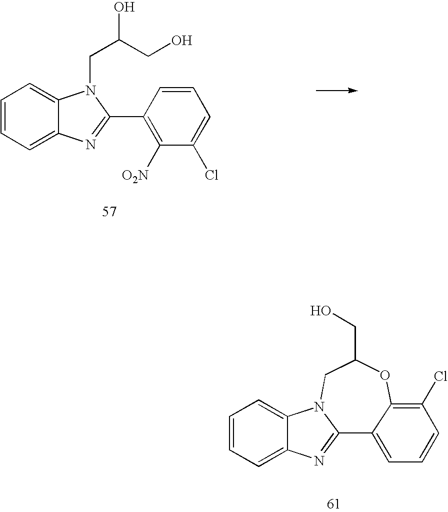

To a solution of diol 57 (100 mg, 0.29 mmol) in anhydrous DMF (2 mL) was added NaH (60% w/w, 25.3 mg, 0.63 mmol). After 21 h at rt, the reaction mixture was quenched with water, and diluted with EtOAc. The organic phase was washed repeatedly with water, dried (MgSO₄), and evaporated in vacuo to give an off-white solid. Purification by flash chromatography (silica gel, CH₂Cl₂→CH₂Cl₂/EtOAc, 1/1) gave the title compound 61 (70 mg, 81%) as a white solid: R_{f}=0.75 (EtOAc); ¹H NMR (400 MHz, d₆-DMSO) δ 3.83 (dd, J=11.0, 5.5 Hz, 1H), 3.92 (dd, J=11.0, 5.0 Hz, 1H), 4.52 (dd, J=14.5, 8.5 Hz, 1H), 4.64 (dddd, J=8.5, 5.5, 5.0, 2.0 Hz, 1H), 4.77 (dd, J=14.5, 2.0 Hz, 1H), 5.27 (br s, 1H), 7.28 (t, J=8.0 Hz, 1H), 7.33 (dt, J=7.0, 1.0 Hz, 1H), 7.37 (dt, J=7.0, 1.0 Hz, 1H), 7.63-7.69 (m, 2H), 7.77 (˜d, J=7.0 Hz, 1H), and 8.46 (dd, J=8.0, 1.5 Hz, 1H); ¹³C{¹H} NMR (101 MHz, d₆-DMSO) δ 46.63, 61.53, 82.10, 110.4, 119.1, 122.1, 122.5, 122.8, 123.7, 125.6, 129.5, 131.5, 136.2, 142.1, 148.9, and 151.2; IR (KBr) νₘₐₓ 1474, 1455, 1427, 1259, 1247, 1099, and 1058 cm⁻¹; MS (ESI) m/z (rel intensity) 301 (100%, MH⁺); HRMS calcd for C₁₆H₁₄ClN₂O₂ (MH⁺) 301.0744, found 301.0728. Alcohol 61 was optically resolved by CSP HPLC (FIG. S6).

### Intermolecular S_{N}Ar Reactions

### 1-Methyl-2-(2-nitrophenyl)-1H-benzimidazole (31)

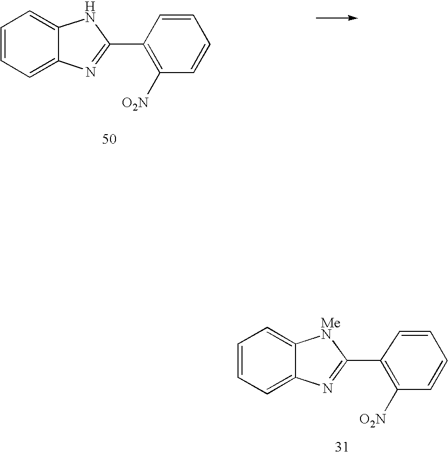

To an ice-cooled suspension of benzimidazole 50^{xviii} (2.00 g, 8.37 mmol) in THF (15 mL) was added NaH (60% w/w, 368 mg, 9.2 mmol) portionwise over 5 min. After 10 min at 0° C., the resulting red solution was treated with MeI (1.3 mL, 21 mmol), and stirred at 0° C.→rt for 16 h. The reaction mixture was quenched with water, evaporated in vacuo, and partitioned between CH₂Cl₂ and water. The phases were separated, and the extraction was completed with additional portions of CH₂Cl₂. The combined organic extracts were dried (MgSO₄), and evaporated in vacuo. Purification by flash chromatography (silica gel, EtOAc/petroleum ether, 1/1) gave the title compound 31 (1.52 g, 72%) as a yellow solid: R_{f}=0.60 (EtOAc); mp 132.5-133.5° C. (EtOAc/petroleum ether) (Lit.^{xxii} 135-137° C.); ¹H NMR (400 MHz, CDCl₃) δ 3.60 (s, 3H), 7.32 (dt, J=7.5, 1.5 Hz, 1H), 7.36 (dt, J=7.5, 1.5 Hz, 1H), 7.41 (dd, J=7.0, 1.5 Hz, 1H), 7.64 (dd, J=8.0, 1.5 Hz, 1H), 7.68 (dt, J=8.0, 1.5 Hz, 1H), 7.75 (dt, J=7.5, 1.0 Hz, 1H), 7.81 (dd, J=7.0, 1.5 Hz, 1H), and 8.18 (dd, J=8.0, 1.0 Hz, 1H); ¹³C{¹H} NMR (101 MHz, CDCl₃) δ 30.39, 109.5, 119.8, 122.3, 123.0, 124.6, 125.8, 130.9, 132.6, 133.4, 135.5, 142.7, 148.5, and 149.6; IR (CHCl₃) νₘₐₓ 1534, 1462, 1438, 1394, and 1348 cm⁻¹; MS (ESI) m/z (rel intensity) 254 (100%, MH⁺) and 207 (25); HRMS calcd for C₁₄H₁₂N₃O₂ (MH⁺) 254.0909, found 254.0906; Anal. Calcd for C₁₄H₁₁N₃O₂: C, 66.40; H, 4.38; N, 16.59. Found: C, 66.69; H, 4.40; N, 16.72.

### 2-(2-Methoxyphenyl)-1-methyl-1H-benzimidazole (32) and 2-(2-methoxyphenyl)-1,3-dimethyl-3H-benzimidazol-1-ium iodide (62)

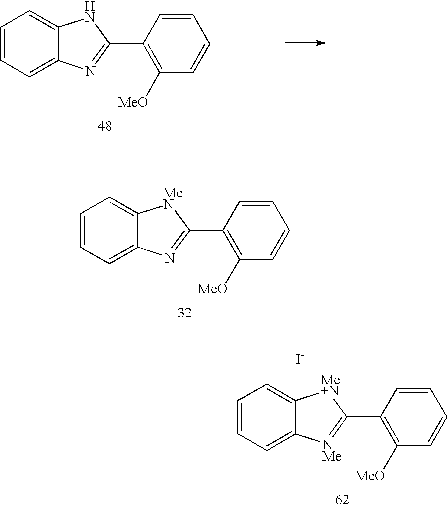

To a solution of benzimidazole 48 (1.00 g, 4.20 mmol) in THF (20 mL) was added NaH (60% w/w, 185 mg, 4.6 mmol) portionwise at 0° C. After 15 min at rt, the resulting red solution was treated with MeI (314 μL, 5.0 mmol), and stirred at rt for 18 h. The reaction mixture was quenched with water, evaporated in vacuo, and partitioned between CH₂Cl₂ and water. The phases were separated, and the extraction was completed with additional portions of CH₂Cl₂. The combined organic extracts were dried (MgSO₄), and evaporated in vacuo to give a white solid. Purification by flash chromatography (silica gel, CH₂Cl₂→CH₂Cl₂/EtOAc, 20/1) gave the title compound 32 (600 mg, 57%) as a clear oil that solidified on standing: R_{f}=0.65 (EtOAc); ¹H NMR (400 MHz, CDCl₃) δ 3.61 (s, 3H), 3.76 (s, 3H), 6.99 (d, J=8.5 Hz, 1H), 7.07 (dt, J=7.5, 0.5 Hz, 1H), 7.22-7.30 (m, 2H), 7.32-7.38 (m, 1H), 7.46 (dt, J=8.5, 1.5 Hz, 1H), 7.56 (dd, J=7.5, 1.5 Hz, 1H), and 7.77-7.82 (m, 1H); ¹³C{¹H} NMR (101 MHz, CDCl₃) δ 31.28, 55.97, 109.9, 111.5, 120.1, 120.2, 121.4, 122.3, 122.8, 132.0, 132.8, 136.5, 143.6, 152.6, and 158.0; IR (CHCl₃) νₘₐₓ 1609, 1477, 1463, 1439, 1388, and 1254 cm⁻¹; MS (ESI) m/z (rel intensity) 239 (100%, MH⁺); HRMS calcd for C₁₅H₁₅N₂O (MH⁺) 239.1184, found 239.1161.

When an analogous reaction was performed with an excess of MeI (2.5 equiv.), only a small amount (˜10%) of benzimidazole 32 was isolated. Instead, the benzimidazolium salt 62 was obtained as the major product (77%). Salt 62: a white solid: mp>260° C. (EtOAc); ¹H NMR (250 MHz, d₄-MeOH) δ 3.81 (s, 6H), 3.84 (s, 3H), 7.23 (dt, J=7.5, 1.5 Hz, 1H), 7.33 (d, J=8.5 Hz, 1H), 7.60-7.69 (m, 3H), 7.75 (ddd, J=8.5, 7.5, 1.5 Hz, 1H), and 7.86-7.92 (m, 2H); ¹³C{¹H} NMR (101 MHz, d₄-MeOH) δ 33.32, 56.98, 110.4, 113.8, 114.2, 122.7, 128.2, 133.1, 133.5, 136.9, 150.2, and 159.6; IR (KBr) νₘₐₓ 1603, 1582, 1516, 1487, 1470, 1446, 1435, and 1259 cm⁻¹; MS (ESI) m/z (rel intensity) 253 (100%, M-I⁻), 237 (80), and 221 (7); HRMS calcd for C₁₆H₁₇N₂O (M-I⁻) 253.1341, found 253.1325.

### 2-(2-Methoxyphenyl)-1-methyl-1H-benzimidazole (32)

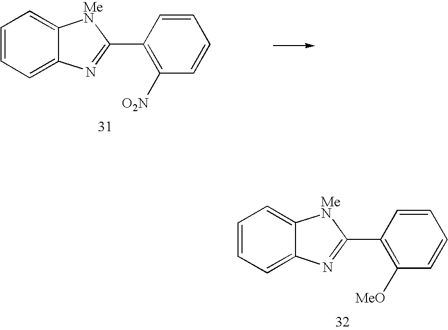

To a solution of benzimidazole 31 (100 mg, 0.40 mmol) in anhydrous DMF (2 mL) was added MeONa (213 mg, 3.95 mmol). After 20 h at 100° C., the reaction mixture was cooled to rt, and partitioned between EtOAc and water. The organic layer was washed repeatedly with water, dried (MgSO₄), and evaporated in vacuo to give a yellow solid. Purification by flash chromatography (silica gel, CH₂Cl₂→CH₂Cl₂/EtOAc, 3/1) gave an inseparable ˜1.9:1 mixture (87 mg) of the starting material 31 and the title compound 32 as a yellow oil. When an analogous reaction was carried out at rt for 25 h, it gave a ˜3.3:1 mixture (97 mg) of the starting material 31 and the title compound 32.

### 1-Methyl-2-(4-nitrophenyl)-1H-benzimidazole (33)

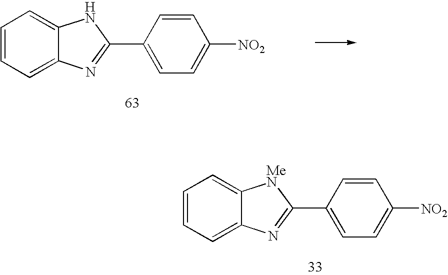

To a suspension of benzimidazole 63^{xxiii} (2.00 g, 8.37 mmol) in THF (15 mL) was added NaH (60% w/w, 368 mg, 9.2 mmol) portionwise at 0° C. After 15 min at rt, the resulting red solution was treated with MeI (1.3 mL, 21 mmol), and stirred at rt for 19 h. The reaction mixture was quenched with water, evaporated in vacuo, and partitioned between CH₂Cl₂ and water. The phases were separated, and the extraction was completed with additional portions of CH₂Cl₂. The combined organic extracts were dried (MgSO₄), and evaporated in vacuo. Purification by flash chromatography (silica gel, CH₂Cl₂→CH₂Cl₂/EtOAc, 20/1) gave the title compound 33 (1.47 g, 69%) as a yellow solid: R_{f}=0.75 (EtOAc/CH₂Cl₂, 1/1); mp 208.0-209.0° C. (EtOAc/petroleum ether) (Lit.^{xxiv} 211-213° C.); ¹H NMR (400 MHz, CDCl₃) δ 3.91 (s, 3H), 7.34 (dt, J=7.0, 1.0 Hz, 1H), 7.37 (˜t, J=7.0 Hz, 1H), 7.42 (dd, J=7.5, 1.5 Hz, 1H), 7.83 (dd, J=7.5, 1.5 Hz, 1H), 7.98 (d, J=8.5 Hz, 2H), and 8.37 (d, J=8.5 Hz, 2H); ¹³C{¹H} NMR (101 MHz, CDCl₃) δ 31.92, 109.9, 120.3, 123.1, 123.8, 123.9, 130.3, 136.3, 136.7, 142.8, 148.4, and 151.0; IR (CHCl₃) νₘₐₓ 1604, 1527, and 1350 cm⁻¹; MS (ESI) m/z (rel intensity) 254 (100%, MH⁺) and 208 (20); HRMS calcd for C₁₄H₁₂N₃O₂ (MH⁺) 254.0929, found 254.0936; Anal. Calcd for C₁₄H₁₁N₃O₂: C, 66.40; H, 4.38; N, 16.59. Found: C, 66.49; H, 4.41; N, 16.66.

### 2-(4-Methoxyphenyl)-1-methyl-1H-benzimidazole (34)

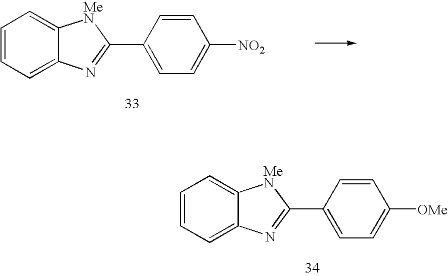 To a suspension of benzimidazole 33 (100 mg, 0.40 mmol) in anhydrous DMF (2 mL) was added MeONa (213 mg, 3.95 mmol). After 18 h at 100° C., the reaction mixture was cooled to rt, and partitioned between EtOAc and water. The organic layer was washed repeatedly with water, dried (MgSO₄), and evaporated in vacuo to give a yellow solid. Purification by flash chromatography (silica gel, CH₂Cl₂→CH₂Cl₂/EtOAc, 20/1) gave the recovered starting material 33 (42 mg, 42%), and the title compound 34 (53 mg, 56%) as a white solid. When an analogous reaction was carried out at rt for 24 h, it gave the recovered starting material 33 (73 mg, 73%) and the title compound 34 (23 mg, 24%). Benzimidazole 34: R_{f}=0.25 (EtOAc/petroleum ether, 1/1); ¹H NMR (400 MHz, CDCl₃) δ 3.84 (s, 3H), 3.89 (s, 3H), 7.06 (˜dt, J=9.0, 2.5 Hz, 2H), 7.28-7.39 (m, 3H), 7.73 (˜dt, J=9.0, 2.5 Hz, 2H), and 7.82-7.86 (m, 1H); ¹³C{¹H} NMR(63 MHz, CDCl₃) δ 31.58, 55.28, 109.4, 114.0, 119.4, 122.2, 122.3, 122.4, 130.7, 136.4, 142.7, 153.6, and 160.7; IR (CHCl₃) νₘₐₓ 1614, 1485, 1462, 1436, 1384, 1253, and 1176 cm⁻¹; MS (ESI) m/z (rel intensity) 239 (100%, MH⁺); HRMS calcd for C₁₅H₁₅N₂O (MH⁺) 239.1184, found 239.1171.

### EXAMPLE 1

The procedure used in the NCI's test for agents active against HIV is designed to detect agents acting at any stage of the virus reproductive cycle. The assay basically involves the killing of T4 lymphocytes by HIV. Small amounts of HIV are added to cells, and two cycles of virus reproduction are necessary to obtain the required cell killing. Agents that interact with virions, cells, or virus gene-products to interfere with vital activities will protect cells from cytolysis. All tests are done with at least one positive (e.g., AZT-treated) control done at the same time under identical conditions. The procedure is as follows:

Candidate agent was dissolved in dimethyl sulfoxide then diluted 1:100 in cell culture medium before preparing serial half-log₁₀ dilutions. T4 lymphocytes (CEM cell line) were added and after a brief interval HIV-1 was added, resulting in a 1:200 final dilution of the compound. Uninfected cells with the compound served as a toxicity control, and infected and uninfected cells without the compound served as basic controls.

Cultures were incubated at 37° in a 5% carbon dioxide atmosphere for 6 days.

The tetrazoleum salt, XTT, was added to all wells, and cultures were incubated to allow formazen color development by viable cells.

Individual wells were analyzed spectrophotometrically to quantitate formazan production, and in addition were viewed microscopically for detection of viable cells and confirmation of protective activity.

Drug-treated virus-infected cells were compared with drug-treated noninfected cells and with other appropriate controls on the same plate.

Data were reviewed in comparison with other tests done at the same time and a determination about activity was made.

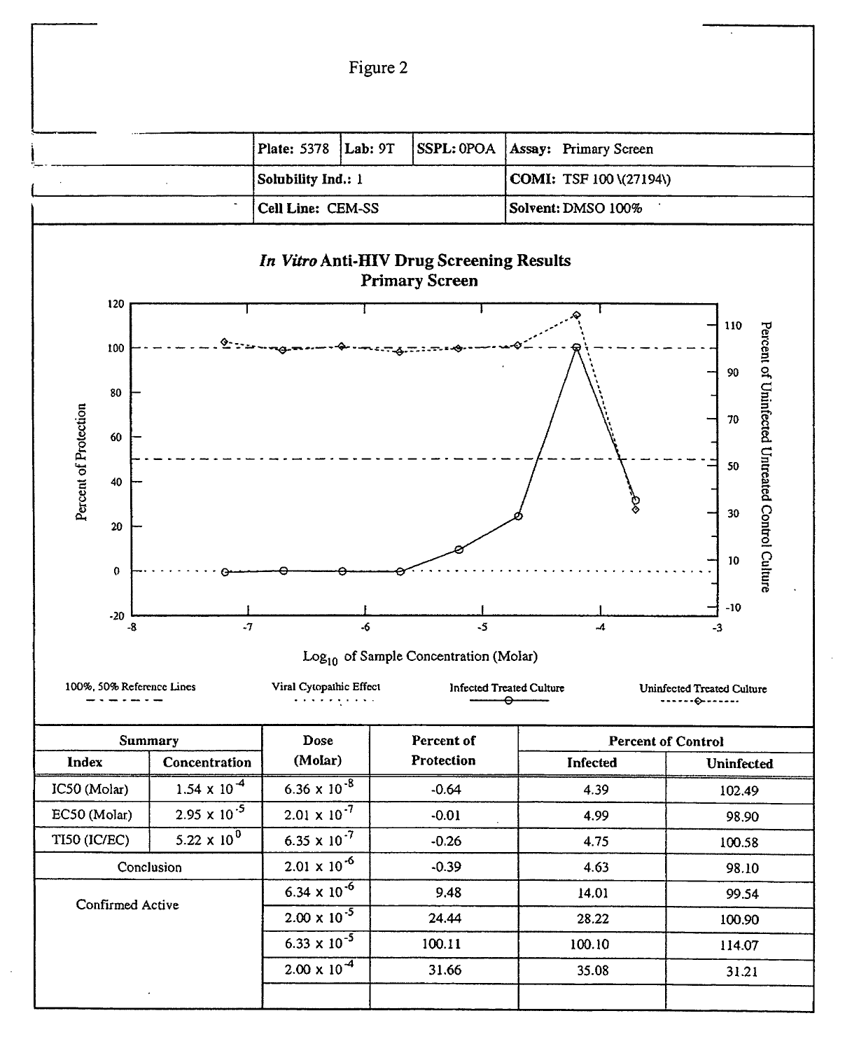

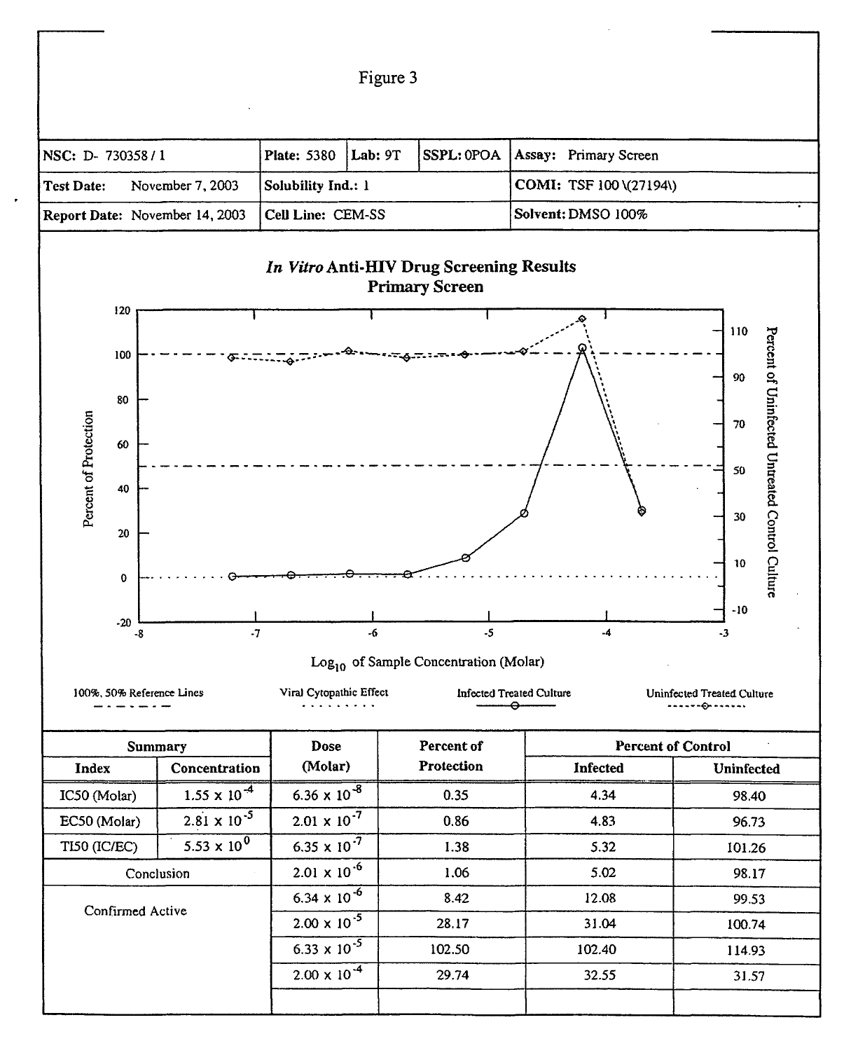

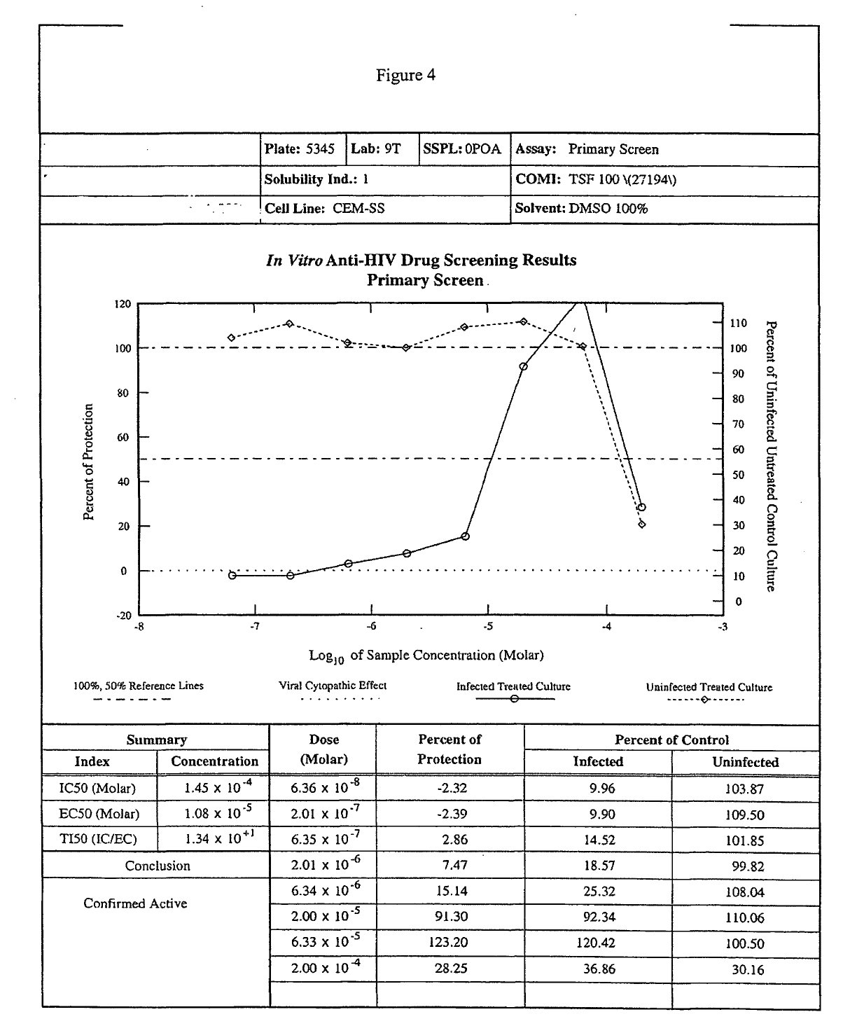

The compound below was tested according to the procedure outlined above. The compound was confirmed active against HIV. The results are shown in FIGS. 1-4.

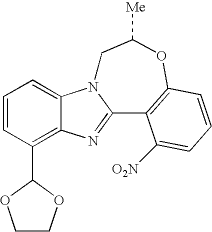

The figures display a plot of the log₁₀ of the sample's concentrations (as μg/mL or M) against the measured test values expressed as a percentage of the uninfected, untreated control values. The solid line depicts the percentage of surviving HIV-infected cells treated with the sample (at the indicated concentration) relative the uninfected, untreated controls. This line expresses the in vitro anit-HIV activity of your sample. The dashed line depicts the percentage of surviving uninfected cells treated with your sample relative to the same, uninfected, untreated controls. This line expresses the in vitro growth inhibitory properties of your sample. The viral cytopathic effect in this particular experiment is indicated by a dotted reference line. This line shows the extent of destruction of cells by the virus in absence of treatment and is used as a quality control parameter. Survival values of this parameter less than 50% are considered acceptable in the current protocol. The percent protection has been calculated from the data and is presented on the left side of the graph.

The Tabular dose response data and status section provides a listing of the numerical data plotted in the graphics section. Approximate values for 50% effective concentration (EC₅₀) against HIV cytopathic effects, 50% inhibitory concentration (IC₅₀) for cell growth, and Therapeutic Index (TI=IC₅₀/EC₅₀) have been calculated for each test and are provided. As shown in the lower left corner, the compound was found active against HIV.

The examples and methods described herein are for illustration only and not meant to limit the invention in any way.
ⁱ (a) Payra, P.; Hung, S.-C.; Kwok, W.-H.; Johnston, D.; Gallucci, J.; Chan, M. K. Inorg. Chem. 2001, 40, 4036-4039. (b) Kwok, W.-H.; Zhang, H.; Payra, P.; Duan, M.; Hung, S.-C.; Johnston, D. H.; Gallucci, J.; Skrzypczak-Jankun, E.; Chan, M. K. Inorg. Chem. 2000, 39, 2367-2376. (c) Payra, P.; Zhang, H.; Kwok, W.-H.; Duan, M.; Gallucci, J.; Chan, M. K. Inorg. Chem. 2000, 39, 1076-1080.
ⁱⁱ Fekner, T.; Gallucci, J.; Chan, M. K. Submitted for publication.
ⁱⁱⁱ (a) Pratt, W. B. Chemotherapy of Infection; Oxford University Press: New York, 1977. (b) White, A. W.; Almassy, R.; Calvert, A. H.; Curtin, N. J.; Griffin, R. J.; Hostomsky, Z.; Maegley, K.; Newell, D. R.; Srinivasan, S.; Golding, B. T. J. Med. Chem. 2000, 43, 4084-4097. (c) Bostock-Smith, C. E.; Searle, M. S. Nucleic Acid Res. 1999, 27, 1619-1624. (d) Roth, T.; Morningstar, M. L.; Boyer, P. L.; Hughes, S. H.; Buckheit, Jr., R. W.; Michejda, C. J. J. Med. Chem. 1997, 40, 4199-4207.
^{iv} Twieg, R.; Matray, T.; Hedrick, J. L. Macromolecules 1996, 29, 7335-7341.
^{v} Most of the alcohols used in these studies were prepared by the Cu(OTf)₂ catalyzed ring opening of epoxides with 1-unsubstituted benzimidazoles. This reaction had previously been successfully applied to the epoxide ring opening with poorly nucleophilic nitroanilines, see: Sekar, G.; Singh, V. K. J. Org. Chem. 1999, 64, 287-289.
^{vi} Typical ^{Experimental} Procedure (Table 1, entry 3): To a solution of alcohol 8 (100 mg, 0.29 mmol) in anhydrous DMF (2 mL) was added NaH (60% w/w, 12.8 mg, 0.32 mmol) to give a dark-green solution. After 11 h at rt, the reaction mixture was quenched with water, and diluted with EtOAc. The organic phase was washed repeatedly with water, dried (MgSO₄), and evaporated in vacuo to give a yellow solid. Purification by flash chromatography (silica gel, CH₂Cl₂→CH₂Cl₂/EtOAc, 10/1) gave the title compound 9 (74 mg, 86%) as a white solid.
^{vii} For a recent controversy concerning the ring current effects on ¹H NMR chemical shifts, see: Wannere, C. S.; Schleyer, P. v. R. Org. Lett. 2003, 5, 605-608.
^{viii} Diols 20 and 24 were prepared by Sharpless asymmetric dihydroxylation of the corresponding N-allyl-substituted benzimidazoles. ¹H NMR analysis of their Mosher diesters (Dale, J. A.; Dull, D. L.; Mosher, H. S. J. Org. Chem. 1969, 34, 2543-2549) indicated that the parent diols 20 and 24 were virtually racemic (ee<5%).
^{ix} The identity of the two compounds 25 and 26 was elucidated by ¹H NMR analysis, as their D₂O-exchangeable primary and secondary hydroxyl groups, respectively, gave the anticipated splitting patterns. In addition, the aromatic proton located ortho to the aryl-heteroaryl axis in the seven-membered cyclic ether 25 experiences a far greater downfield shift than the analogous proton in the rotationally less restricted eight-membered cyclic ether 26 (8.44 and 7.74 ppm, respectively). The identity of the hydroxymethyl compound 25 was further confirmed by its conversion, via the corresponding tosylate, into its methyl analogue 27.
^{x} Bartoli, G.; Todesco, P. E. Acc. Chem. Res. 1977, 10, 125-132.
^{xi} When 5 was treated with NaH in DMF for 18 h at 90° C., alkenes 30 (E/Z=˜2:1) were formed in high yield (85%). Treatment of alcohol 4 with excess NaH (2.2 equiv.) for 24 h at rt→90° C. also gave alkenes 30 (71%, E/Z=˜2.2:1). For sterically more demanding secondary alcohols (e.g., 10), the post-S_{N}Ar isomerization step is much slower, requiring higher temperatures and more prolonged reaction times, and leads to formation of isomeric alkenes with a higher E/Z ratio.
^{xii} It is frequently observed in S_{N}Ar processes that a nitro group located ortho to an activator is replaced more readily than a para-positioned group (Bendedetti, F.; Marshall, D. R.; Stirling, C. J. M.; Leng, J. L. Chem. Commun. 1982, 918-919), as the former is more likely to be out-of-plane relative to the aromatic ring. Therefore, formation of an intermediate Meisenheimer complex is expected to disturb the aromaticity of the molecular system to a lesser degree. However, 33 is a superior substrate, especially at elevated temperatures, than its analogue 31.
^{xiii} Perrin, D. D.; Armarego, W. L. F. Purification of Laboratory Chemicals; Pergamon Press: New York, 1988.
^{xiv} Still, W. C.; Hahn, M.; Mitra, A. J. Org. Chem. 1978, 43, 2923-2925.
^{xv} Vanelle, P.; Liegeois, C. T.; Meuche, J.; Maldonado, J.; Crozet, M. P. Heterocycles 1997, 45, 955-962.
^{xvi} Most peaks doubled due to the NH-tautomerizm.
^{xvii} Nardi, D.; Tajana, A.; Rossi, S. J. Het. Chem. 1973, 10, 815-819.
^{xviii} Zaika, L. L.; Joullié, M. M. J. Het. Chem. 1966, 3, 289-298.
^{xix} As the product distribution in N-alkylations of unsymmetrical 2-aryl-1H-benzimidazoles (e.g., 39) is usually strongly biased towards a sterically less hindered of the two possible isomers, the major product 53 was assigned the structure as depicted, with the allyl substituent attached to the sterically more accessible benzimidazole nitrogen.
^{xx} Alcohols 1 and 12 were prepared as part of our studies on the synthesis of optically pure, strapped cyclic bis(benzimidazole) ligands. Results of these studies will be reported in due course.
^{xxi} In the presence of D₂O, the splitting pattern for the signals at 3.78 and 3.88 ppm changed from ‘ddd’ to ‘dd’.
^{xxii} Hawkins, D.; Lindley, J. M.; McRobbie, I. M.; Meth-Cohn, O. J. Chem. Soc., Perkin Trans. 1, 1980, 2387-2391.
^{xxiii} Prabhakar Reddy, V.; Prasunamba, P. L.; Reddy, P. S. N.; Ratnam, C. V. Ind. J. Chem. Sect. B 1983, 22, 917-918.
^{xxiv} El'tsov, A. V.; Muravich-Aleksandr, Kh. L. J. Org. Chem. USSR (Engl.) 1965, 1321-1327.

1. A compound of formula I: 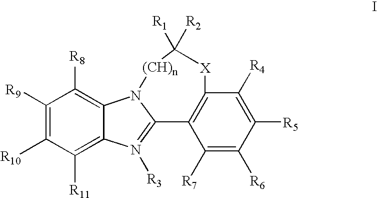
wherein:
R₁ and R₂ are the same or different and are selected from the group consisting of H, alkyl, cycloalkyl, alkenyl, alkynyl, hydroxyl, halide, nitro, carboxylate, amino, amido, epoxide, and labeling reagents;
R₃ is optional and is selected from the group consisting of an oxo, a terminal epoxide, alkyl, branched alkyl, cycloalkyl, hydroxyl, halide, nitro, carboxylate, amido, epoxide, amino, substituted amino, aryl, or vinyl group;
R₄-R₇ are the same or different and are selected from the group consisting of H, alkyl, cycloalkyl, hydroxyl, halide, nitro, carboxylate, amido, epoxide, amino, substituted amino, aryl, vinyl, acetal, aldehyde, and labeling reagents;
R₈-R₁₁ are the same or different and are selected from the group consisting of H, linear alkyl, branched alkyl, cycloalkyl, hydroxyl, halide, nitro, carboxylate, amido, epoxide, amino, substituted amino, aryl, acetal, aldehyde, and vinyl;
X is a heteroatom selected from O, S, Se, NH, PH, AsCH₂; and
n is 0-5;
or a derivative, or metabolite thereof.

2. The compound of claim 1 wherein R₃ is null.

3. The compound of claim 1 wherein any of R₁-R₇ are a labeling reagent, and the labeling reagent is selected from the group consisting of wherein the labeling reagent is selected from the group consisting of biotin, coumarin and fluoroscene dyes.

4. The compound of claim 1, wherein the compound is a derivative of formula I, wherein the benzimidazole of formula 1 has been replaced by a functional group selected from the group consisting of imidazole, imidazoline, pyrrole, or pyrrolidine, benzoxazole, and indole.

5. The compound of claim 4, wherein the compound is a derivative of formula II: 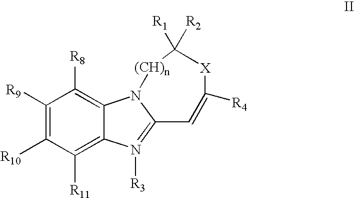
wherein:
R₄ is selected from the group consisting of H, alkyl, cycloalkyl, hydroxyl, halide, nitro, carboxylate, amido, epoxide, amino, substituted amino, aryl, vinyl, acetal, aldehyde, and labeling reagents; or a derivative, or metabolite thereof.

6. The compound of claim 4, wherein the compound is a derivative of formula IIIA or IIIB: 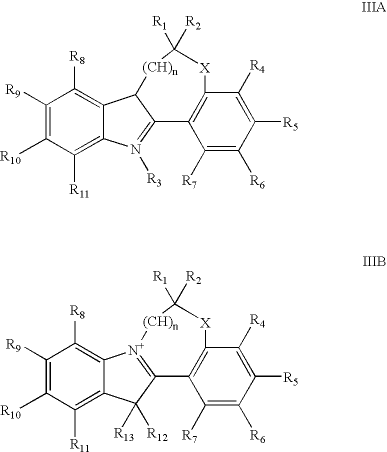
wherein R₁₂ and R₁₃ are selected from the group consisting of H, linear alkyl, branched alkyl, cycloalkyl, hydroxyl, halide, nitro, carboxylate, amido, epoxide, amino, and substituted amino group; or a derviative or metabolite thereof.

7. The compound of claim 4, wherein the compound is a derivative of formula IV: 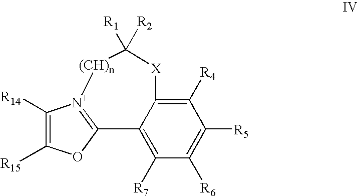
wherein R₁₄ and R₁₅ are the same or different and are selected from the group consisting of H, alkyl, cycloalkyl, hydroxyl, halide, nitro, carboxylate, amido, epoxide, or amino; or a derivative or metabolite thereof.

8. The compound of claim 4, wherein the compound is a derivative of formula V: 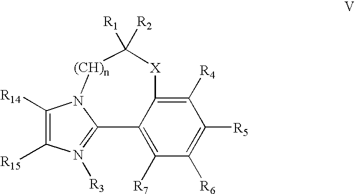
wherein R₁₄ and R₁₅ are the same or different and are selected from the group consisting of H, alkyl, cycloalkyl, hydroxyl, halide, nitro, carboxylate, amido, epoxide, or amino; or a derivative or metabolite thereof.

9. The compound of claim 4, wherein the compound is a derivative of formula VI: 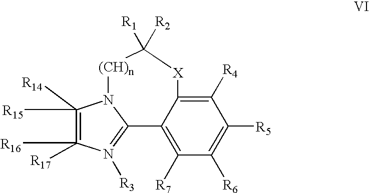
wherein R₁₄-R₁₇ are the same or different and are selected from the group consisting of H, alkyl, cycloalkyl, hydroxyl, halide, nitro, carboxylate, amido, epoxide, or amino; or a derivative or metabolite thereof.

10. The compound of claim 4, wherein the compound is a derivative of formula VII: 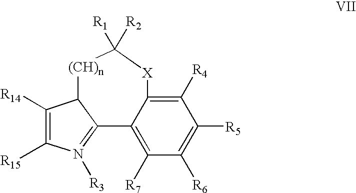
wherein R₄ and R₁₅ are the same or different and are selected from the group consisting of H, alkyl, cycloalkyl, hydroxyl, halide, nitro, carboxylate, amido, epoxide, or amino; or a derivative or metabolite thereof.

11. The compound of claim 4, wherein the compound is a derivative of formula VIII: 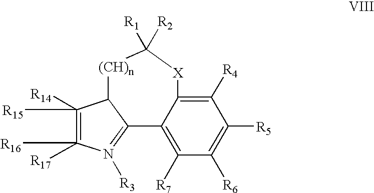
wherein R₁₄ and R₁₅ are the same or different and are selected from the group consisting of H, alkyl, cycloalkyl, hydroxyl, halide, nitro, carboxylate, amido, epoxide, or amino; or a derivative or metabolite thereof.

12. The compound of claim 1, wherein the compound is

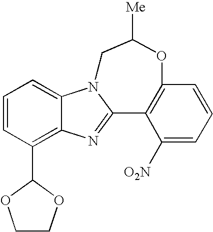

13. A method for preparing a compound of claim 1, comprising the steps
a) selecting a starting compound of formula IX or a derivative thereof 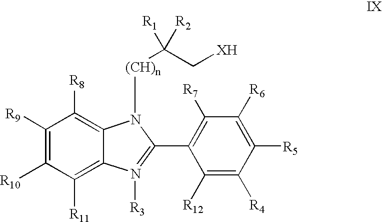
b) contacting the compound of formula IX with NaH under mild conditions for a sufficient period of time for an intramolecular aromatic nucleophilic substitution (S_{N}Ar) to occur; whereby an S_{N}Ar product of formula 1, or a derivative thereof, is formed;
wherein:
R₁ and R₂ are the same or different and are selected from the group consisting of H, alkyl, cycloalkyl, alkenyl, alkynyl, hydroxyl, halide, nitro, carboxylate, amino, amido, epoxide, and labeling reagents;
R₃ is optional and is selected from the group consisting of an oxo, a terminal epoxide, alkyl, branched alkyl, cycloalkyl, hydroxyl, halide, nitro, carboxylate, amido, epoxide, amino, substituted amino, aryl, or vinyl group;
R₄-R₇ are the same or different and are selected from the group consisting of H, alkyl, cycloalkyl, hydroxyl, halide, nitro, carboxylate, amido, epoxide, amino, substituted amino, aryl, vinyl, acetal, aldehyde, and labeling reagents;
R₈-R₁₁ are the same or different and are selected from the group consisting of H, linear alkyl, branched alkyl, cycloalkyl, hydroxyl, halide, nitro, carboxylate, amido, epoxide, amino, substituted amino, aryl, acetal, aldehyde, and vinyl;
R₁₂ is selected from the group consisting of —NO₂, F, Cl, Br, OTs, SOPH, and N₃;
X is a heteroatom selected from O, S, Se, NH, PH, AsCH₂; and
n is 0-5;
or a derivative thereof.

14. The method of claim 11 wherein the benzimidazole of formula IX is replaced with a functional group selected from the group consisting of imidazole, imidazoline, pyrrole, or pyrrolidine, benzoxazole, and indole.

15. The method of claim 13 comprising the additional steps of
a) adding excess NaH; and
b) heating the compound of formula IX and excess NaH for a period of time sufficient to convert the S_{N}Ar product to a corresponding benzimidazole.

16. A method of treating a subject infected with HIV comprising the step of administering a therapeutically effective amount of a compound of claim 1 to a subject in need of such treatment.

17. The method of claim 16 wherein the compound is 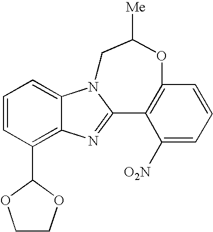
or a metabolite or prodrug thereof.

18. The method of claim 16 wherein the subject is a human subject.

19. A method of fabricating an opto-electrical device, comprising the steps of
a) selecting a compound of claim 1, wherein the compound is a homochiral tetracyclic compound;
b) coupling the homochiral tetracylic compound into a polymer that can form a self-assembled monolayer; and
c) forming the self-assembled monolayer to fabricate the opto-electrical device.

20. The opto-electrical device formed by the process of claim 19.
